(19) United States
(12) Patent Application Publication
(10) Pub. No.: US 2010/0190795 A1
(43) Pub. Date: Jul. 29, 2010

(54) BETULIN DERIVED COMPOUNDS USEFUL AS ANTIPROTOZOAL AGENTS

(75) Inventors: Jari Yli-Kauhaluoma, Helsinki (FI); Sami Alakurtti, Vantaa (FI); Jaana Minkkinen, Espoo (FI); Nina Sarcerdoti-Sierra, Jerusalem (IL); Charles L. Jaffe, Yavne (IL); Tuomo Heiska, Helsinki (FI)

Correspondence Address: BIRCH STEWART KOLASCH & BIRCH, PO BOX 747, FALLS CHURCH, VA, 22040-0747, US

(73) Assignee: Valtion Teknillinen tutkimuskeskus, Espoo (FI)

(21) Appl. No.: 12/303,707

(22) PCT Filed: Jun. 6, 2007
(86) PCT No.: PCT/FI07/50331

§ 371 Date: Jul. 24, 2009

(30) Foreign Application Priority Data
Jun. 7, 2006 (FI) 20065388

(51) Int. Cl. A01N 43/58; C07C 69/34; A01N 37/02; A01P 15/00; C07D 213/30; A01N 43/40; C07D 487/02

(52) U.S. Cl. 514/248; 560/194; 514/510; 560/61; 546/285; 514/356; 544/236; 544/233

(57) ABSTRACT

The invention relates to betulin derivatives, and to the use thereof as agents against protozoa of the genus Leishmania and against leishmaniasis in applications of pharmaceutical industry.

### FIELD OF THE INVENTION

The invention relates to compounds derived from betulin, and to the therapeutic use thereof in applications of pharmaceutical industry, particularly as agents against protozoa of Leishmania genus and leishmaniasis caused by said protozoa. Further, the invention relates to novel betulin derivatives and methods for the production thereof, either directly from betulin, or from intermediates derived therefrom.

### STATE OF THE ART

Betulin having the structure 1 shown below is a naturally occurring pentacyclic triterpene alcohol of the lupane family, also known as betulinol and lup-20(29)-ene-3β,28-diol. Betulin is found in the bark of some tree species, particularly in the birch (Betula sp.) bark at best in amounts up to 40% of the bark dry weight. In addition to betulin, also minor amounts of betulin derivatives are obtained from tree bark. There are known methods mainly based on extraction for the isolation of betulin from bark material.

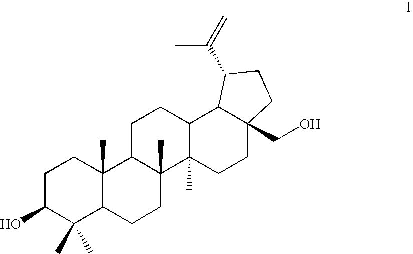

In some applications, poor solubility of betulin causes problems with respect to use and formulation, and accordingly, betulin is converted to its derivatives to improve the solubility. In the production of said derivatives, reactivities of the functional groups of betulin, that is, the primary and secondary hydroxyl groups and the double bond are typically utilized. Both hydroxyl groups may be esterified, thus obtaining mono- or diesters. Glycoside derivatives may be produced from betulin using known procedures, and betulin may be subjected to oxidation, reduction and rearrangement reactions in the presence of a suitable oxidation reagent, reducing reagent, or an acid catalyst, respectively.

Betulinic acid having the structure 3 shown in the reaction scheme below may be isolated e.g. from birch (Betula sp.) bark or cork of cork oak (Quercus suber L.) by extraction, and further, it may be produced by several methods mainly based on direct oxidation of the betulin or birch bark material. The reaction scheme shows the direct oxidation of betulin 1 according to U.S. Pat. No. 6,280,778 as Jones oxidation in the presence of a chromium(VI) oxide catalyst to give betulonic acid 2, followed by the selective reduction of the betulonic acid 2 thus obtained with sodium borohydride to give betulinic acid 3.

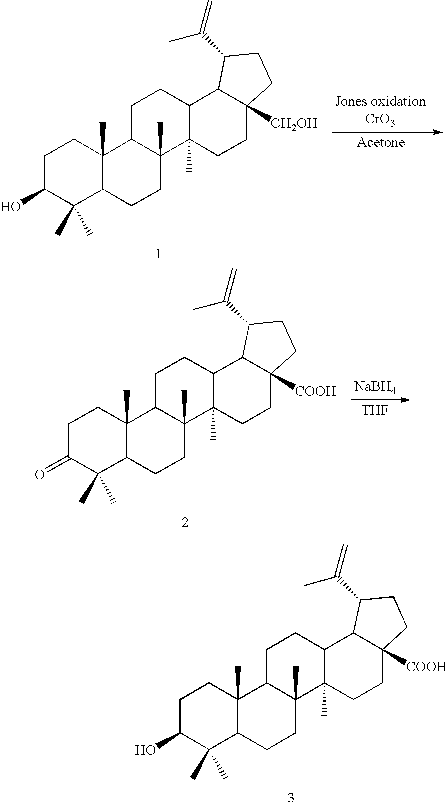

An alternative process for the production of betulinic acid is disclosed in U.S. Pat. No. 5,804,575, comprising an oxidation step where the 3-beta-hydroxyl of betulin is protected by acetylation. Isomerization and oxidation of the secondary hydroxyl group of betulin is thus prevented.

Suitability of betulin and the derivatives thereof for medical and cosmetic applications and for industrial chemical applications is known to some extent. Use of betulin and betulinic acid in cosmetic applications such as promoters of hair growth and thickness and as components in skin creams is already known for instance from WO 0003749. The publication WO 0174327 discloses the use of betulinic acid in sun creams for the prevention of detrimental effects of the UV light. Antitumoral activity of betain particularly against melanoma has been described for instance in U.S. Pat. No. 5,869,535.

Use of betulin and some derivatives thereof as antifungal and anti-yeast agents is described in U.S. Pat. No. 6,642,217.

In U.S. Pat. No. 5,750,587, antiviral activity of betulin and derivatives thereof, particularly against Herpes simplex is discussed.

Antibacterial properties of betulin and several derivatives thereof are presented in WO 026762 (US 2002/0119935). Said compounds are particularly effective against the bacteria Escherichia coli, Staphylococcus aureus and Enterococcus faecalis.

Leishmaniasis is typically a disease occurring in the Mediterranean and tropical countries, caused by protozoa belonging to flagellates and transmitted from animals by sand fly (Phlebotomus spp.). Leishmaniasis is known as cutaneous leishmaniasis (l. cutanea) characterized by persisting skin lesions at the bite sites of the sand fly; mucous and cutaneous leishmaniasis or espundia (l. mucocutanea) spreading on nasal and oral mucous membranes, progressively destroying soft tissues of nose and mouth; and visceral leishmaniasis or kala-azar, a general disease due to infection of the reticuloendothelial system. It is characterized i.e., by fever, anemia, degeneration of tissues and enlargement of liver and spleen.

For instance, Leishmania brasiliensis is the causative agent of mucous and cutaneous leishmaniasis, L. donovani is the causative agent of visceral leishmaniasis, and L. mexicana and L. tropica are the causative agent of cutaneous leishmaniasis. Millions of people are affected by leishmaniasis at least in 88 different countries.

Sauvain M. et al. in Phytother. Res. 1996, 10, 1-4, presents the leishmaniacidal activity against amastigots of the L. amazonensis species, of betulin, betulinic acid and betulinic aldehyde isolated in extremely low amounts from the liana growing in Amazonian rain forests [Doliocarpus dentatus (Aubl.) Standl.] in an in vitro test. Hunters used the nectar of this plant to still their thirst when no drinking water was available. Moreover, indigenous people of Surinam have used powders made from the bark of said plant to heal lesions caused by leishmaniasis.

Takahashi M, et al. in Phytother. Res. 2004, 18, 573-578, describe the leishmanicidal activity against L. major promastigots, of compounds isolated from plants of the Betula genus in an in vitro test.

Antimonium salts such as N-methylglucamine antimonate and sodium stibogluconate are at present used to combat the protozoa of the genus Leishmania, said compounds being typically expensive and toxic in high amount. Is has also been reported that different protein kinases play a significant role in the differentiation of Leishmania species.

On the basis of the above, it is clear that there is an obvious need for novel, potent and safe agents against the protozoa causing leishmaniasis and leishmanicidal agents with only minor side effects.

Betulin and betulinic acid are compounds that may be dissolved, emulsified and/or formulated in water only with difficulty, and poorly converted into preparations for instance for pharmaceutical industry. Thus, there is an obvious need to provide environmentally acceptable novel betulin derivatives having an improved emulsifiability and/or solubility in water or in solvents or media typically used in pharmaceutical applications, said derivatives being very suitable for the production of stable preparations also having desired activities.

Compounds derived from betulin refer here to such betulin derivatives as pentacyclic triterpenoids, betulonic acid and betulin derivatives comprising natural compounds and/or compounds with known low toxicity as substituents, and especially to alcohol, phenol and/or carboxylic acid and/or ester and/or amide and/or ether derivatives of betulin and/or derivatives having a partial heterocyclic structure and/or carbamate derivatives.

### OBJECTS OF THE INVENTION

An object of the invention is the use of compounds derived from betulin as agents against the protozoa of the genus Leishmania causing leishmaniasis and as agents against leishmaniasis.

Another object of the invention is to provide novel betulin derivatives useful as agents against the protozoa of the genus Leishmania causing leishmaniasis and as agents against leishmaniasis.

Still another object of the invention is to provide novel betulin derivatives comprising known naturally occurring compounds, pharmacophoric or other heterocyclic moieties and/or compounds with low toxicity as substituents.

Moreover, another object of the invention is to provide novel betulin derivatives having improved solubilities and/or emulsifiabilities in water and/or in solvents or media typically used in pharmaceutical applications, such as fats, oils, alcohols and the like.

Yet another object of the invention is to provide methods for producing said novel betulin derivatives.

Another object of the invention is to provide compositions comprising said novel betulin derivatives.

Characteristic features of the betulin derivatives, their use and the compositions and production methods according to the invention are disclosed in the claims.

### GENERAL DESCRIPTION OF THE INVENTION

The present invention is directed to the use of compounds derived from betulin as agents against the protozoa of the genus Leishmania causing leishmaniasis and as as agents against leishmaniasis. The invention is further directed to novel betulin derivatives useful as agents against the protozoa of the genus Leishmania causing leishmaniasis and as agents against leishmaniasis, and compositions comprising said derivatives. The present compounds derived from betulin are particularly suitable for applications of pharmaceutical industry.

The invention is also directed to novel betulin derivatives preferably comprising natural compounds and/or known compounds with low toxicity as substituents such as to alcohol, phenol and/or carboxylic acid and/or ester and/or amide and/or ether derivatives of betulin and/or derivatives with heterocyclic structural moieties and/or carbamate derivatives, particularly to carboxylic acid and ester and amide derivatives of betulin and/or derivatives with partial heterocyclic structures and/or carbamate derivatives. The invention is also directed to the use of betulin derivatives as active agents having improved solubilities and/or emulsifiabilities in solvents or media used in pharmaceutical industries, and further to methods for the production of said novel betulin derivatives.

### DETAILED DESCRIPTION OF THE INVENTION

It was surprisingly found that some compounds derived from betulin, including betulonic, acid, have considerable activity against the protozoa of the genus Leishmania causing leishmaniasis and against leishmaniasis.

Several compounds useful according to the invention comprise natural compounds and/or known compounds with low toxicities as substituents, said inventive compounds thus being safe and environmentally acceptable.

According to the invention, it is also possible to produce novel betulin derivatives potent as active agents against the protozoa of the genus Leishmania and leishmanicidal agents, particularly carboxylic acid and ester and amide derivatives of betulin and/or derivatives comprising heterocyclic structural moieties and/or carbamate derivatives, several of said derivatives having improved solubilities and/or emulsifiabilities in solvents and media used in pharmaceutical industries.

It was also surprisingly found that the active agent is released by some betulin derivatives in a controlled manner for an extended time. This allows for efficient specified administration of the products of the invention.

According to the invention, compounds derived from betulin acting as efficient agents against the protozoa of the genus Leishmania and against leishmaniasis include the following compounds derived from betulin having the general formula I shown below, and pharmaceutically acceptable salts thereof, where in formula I

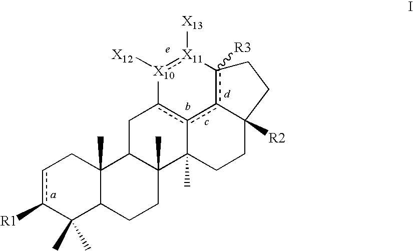

R1=H, —OH, —ORₐ, —O(C═O)R_{b}, —CN, —CHO, —(C═O)ORₙ, —SRₐ, —O(C═O)NHRₐ, ═O or ═S where Rₐ, R_{b} and R_{z} independently represent H, C₁-C₂₂ aliphatic, unbranched or branched, saturated or unsaturated hydrocarbon residue; C₃-C₈ cyclic or heterocyclic residue; substituted or unsubstituted phenyl or benzyl residue; amino, amide or amino acid; substituted or unsubstituted 1,2,3-triazol, 1,2,4-triazol, tetrazol, pyrrole, isoxazol, pyrazol, imidazol, or oxazol; a carboxymethyl, carboxymethylester or carboxymethylamide derivative or a salt thereof;
R2=—CH₂OH, —CH₂ORₐ, —CH₂O(C═O)R_{b}, —(C═O)OR_{b}, —CH₂NRₙR_{z}, —CH₂CN, —CH₂CHO, —CH₂(C═O)ORₐ, —CH₂SRₐ, —CH₂O(C═O)NHRₐ, —CH═O or —CH═S where Rₐ, R_{b} and R_{z} independently represent H, C₁-C₂₂ aliphatic, unbranched or branched, saturated or unsaturated hydrocarbon residue; C₃-C₈ cyclic or heterocyclic residue; substituted or unsubstituted phenyl or benzyl residue; amine, amide or amino acid; substituted or unsubstituted 1,2,3-triazol, 1,2,4-triazol, tetrazol, pyrrole, isoxazol, pyrazol, imidazol, or oxazol; a carboxymethyl, carboxymethylester or carboxymethylamide derivative or a salt thereof;
R3=isopropenyl, isopropyl, isopropylphenyl, isopropylhydroxyphenyl, or isopropylsuccinic acid derivative or a salt thereof;

### X₁₀═X₁₁═H, C or N;

X₁₂═X₁₃=“absent”; (C═O)OR, (C═O)NHR where R═H or a C₁-C₆ linear or branched alkyl or alkenyl group or substituted or unsubstituted phenyl or benzyl residue or X₁₂-X₁₃ forms a cyclic partial structure of the form —(X₁₂═X₁₄)—X₁₅—(X₁₃═X₁₆)— where X₁₂═X₁₃═C, X₁₄═X₁₆=“absent”, O or S, X₁₅═C, O, S or N—X₁₇ where X₁₇═H, C₁-C₆ linear or branched alkyl or alkenyl group, substituted or unsubstituted phenyl or benzyl residue;
a, b, c and d independently represent a double or single bond; and
e=“absent” or represents a double pr single bond; and

Betulin, betulinic acid or betulinic aldehyde are excluded from compounds useful according to the invention.

According to the invention, preferable betulin derivatives include the compounds having the following structures IA-IQ:

### TA:

### R1=OH;

R2=CH₂O(C═O)R_{f} or —CH₂ORₐ(C═O)OR_{f} where R_{f}=C₃-C₈ cyclic or heterocyclic residue, substituted or unsubstituted phenyl or benzyl residue, C₁-C₂₂ linear or branched alkyl or alkenyl group and Rₐ=C₁-C₂₂ linear or branched alkylene or alkenyl group;
R3=CH₂═CCH₃ (isopropenyl group);

### X₁₀═X₁₁═H;

X₁₂═X₁₃=“absent”;
a, b, c, and d each represent a single bond; and
e=“absent”.

### IB:

### R1=OH;

R2=CH₂O(C═O)(CHR_{g})CH₂COOY where R_{g}═H, C₁-C₂₂ linear or branched alkyl or alkenyl group, Y═H, Na, K, Ca, Mg, C₁-C₄-alkyl group, or NRₕ where Rₕ=H or C₁-C₄-alkyl group;

### R3=CH₂═CCH₃;

### X₁₀═X₁₁═H;

X₁₂═X₁₃=“absent”;
a, b, c, and d each represent a single bond; and
e=“absent”.

### IC:

### R1=OH;

R2=CH₂OR₁ where R₁=an ester of ornithine, N-acetylanthranilic acid or trimethylglycine (or betain ester);

### R3=CH₂═CCH₃;

### X₁₀═X₁₁═H;

X₁₂═X₁₃=“absent”;
a, b, c, and d each represent a single bond; and
e=“absent”.

### ID:

### R1=OH;

R2=CH₂O(C═O)CHRⱼ(NHZ) or —CH₂ORₐ(C═O)NHRⱼ where Rₐ=C₁-C₂₂ linear or branched alkylene or alkenyl group; Rⱼ=H, C₁-C₄-alkyl-, benzyl, 4-hydroxybenzyl, CH₂CH₂CH₂CH₂NH₂, 4-imidazolylmethyl or 3-indolylmethyl group, and Z=H, Rₖ, (C═O)Rₖ or COORₖ where Rₖ=C₁-C₂₂ branched or unbranched alkyl or alkenyl group, or a phenyl, benzyl or 4-hydroxybenzyl group; R3=CH₂=CCH₃;

### X₁₀═X₁₁═H;

X₁₂═X₁₃=“absent”;
a, b, c, and d each represent a single bond; and
e=“absent”.

### IE;

### R1=OH;

R2=CH₂ORₙ where Rₙ=an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol, each being carboxymethoxy substituted, or an ester of chrysanthemic acid, cinnamic acid, or retinolic acid;

### R3=CH₂═CCH₃;

### X₁₀═X₁₁═H;

X₁₂═X₁₃=“absent”;
a, b, c, and d each represent a single bond; and
e=“absent”.

### IFa:

R1=O(C═O)Rₘ or —ORₐ(C═O)ORₘ where Rₘ=C₃-C₈ cyclic or heterocyclic residue, substituted or unsubstituted phenyl or benzyl residue, C₁-C₂₂ linear or branched alkyl or alkenyl group and Rₐ=C₁-C₂₂ linear or branched alkylene or alkenyl group;
R2=CH₂O(C═O)Rₒ or —CH₂ORₐ(C═O)Rₒ where Rₒ=C₃-C₈ cyclic or heterocyclic residue, substituted or unsubstituted phenyl or benzyl residue, C₁-C₂₂ linear or branched alkyl or alkenyl group and Rₐ=C₁-C₂₂ linear or branched alkylene or alkenyl group;

### R3=CH₂═CCH₃;

### X₁₀═X₁₁═H;

X₁₂═X₁₃=“absent”;
a, b, c, and d each represent a single bond; and
e=“absent”.

### IFb:

R1=O(C═O)(CHR_{c})CH₂COOY where Rₒ=H, C₁-C₂₂ linear or branched alkyl or alkenyl group, Y═H, Na, K, Ca, Mg, C₁-C₄ alkyl group or NRₕ where Rₕ=H or a C₁-C₄ alkyl group;
R2=CH₂O(C═O)(CHR_{d})CH₂COOY where R_{d}=H, C₁-C₂₂ linear or branched alkyl or alkenyl group, Y═H, Na, K, Ca, Mg, C₁-C₄ alkyl group or NRₖ where Rₖ═H or a C₁-C₄ alkyl group;

### R3=CH₂═CCH₃;

### X₁₀═X₁₁═H;

X₁₂═X₁₃=“absent”;
a, b, c, and d each represent a single bond; and
e=“absent”,

### IFc;

R1=ORᵣ where Rᵣ=an ester of ornithine, N-acetylanthranilic acid, or trimethylglycincee;
R2=CH₂ORₚ where Rₚ=an ester of ornithine ester, N-acetylanthranilic acid, or trimethylglycinee;

### R3=CH₂=CCH₃;

### X₁₀═X₁₁═H;

X₁₂═X₁₃=“absent”;
a, b, c, and d each represent a single bond; and
e=“absent”.

### IFd:

R1=O(C═O)CHRₛ(NHZ) or —ORₐ(C═O)NHRₛ where Rₐ=C₁-C₂₂ linear or branched alkylene or alkenyl group; Rₛ=H, C₁-C₄-alkyl-, benzyl, 4-hydroxybenzyl, CH₂CH₂CH₂CH₂NH₂, 4-imidazolylmethyl or 3-indolylmethyl group, Z═H, Rₖ, (C═O)Rₖ or COORₖ where Rₖ=C₁-C₂₂ branched or unbranched alkyl or alkenyl group, or a phenyl, benzyl or 4-hydroxybenzyl group;
R2=CH₂O(C═O)CHRₓ(NHZ) or CH₂ORₐ(C═O)NHRₓ where Rₐ=C₁-C₂₂ linear or branched alkylene or alkenyl group; Rₓ═H, C₁-C₄-alkyl-, benzyl, 4-hydroxybenzyl, CH₂CH₂CH₂CH₂NH₂, 4-imidazolylmethyl or 3-indolylmethyl group, Z=H, R_{y}, (C═O)R_{y} or COOR_{y} where R_{y}=C₁-C₂₂ branched or unbranched alkyl or alkenyl group, or a phenyl, benzyl or 4-hydroxybenzyl group;

### R3=CH₂═CCH₃;

### X₁₀═X₁₁═H;

X₁₂═X₁₃=“absent”;
a, b, c, and d each represent a single bond; and
e=“absent”.

### IFe;

R1=ORᵥ where Rᵥ=an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol, each being carboxymethoxy substituted, or an ester of chrysanthemic acid, cinnamic acid, or retinolic acid;
R2=CH₂ORₐ where Rᵤ=an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol, each being carboxymethoxy substituted, or an ester of chrysanthemic acid, cinnamic acid, or retinolic acid;

### R3=CH₂═CCH₃;

### X₁₀═X₁₁═H;

X₁₂═X₁₃=“absent”;
a, b, c, and d each represent a single bond; and
e=“absent”.

### IG:

### R1═OH;

R2=(C═O)NHCHRₓCOOY where Y═H, Na, K, Ca, Mg, C₁-C₄ alkyl group or NR_{y} where R_{y}═H or a C₁-C₄ alkyl group, and Rₓ═H, C₁-C₄-alkyl-, benzyl, 4-hydroxybenzyl, CH₂CH₂CH₂CH₂NH₂, 4-imidazolylmethyl or 3-indolylmethyl group or L-aspartate, L-histidine, L-glutamine or L-lysine;

### R3=CH₂═CCH₃;

### X₁₀═X₁₁═H;

X₁₂═X₁₃=“absent”;
a, b, c, and d each represent a single bond; and
e=“absent”.

### IH:

### R1=OH;

R2=(C═O)R_{w} where R_{w}=an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol;

### R3=CH₂═CCH₃;

### X₁₀═X₁₁═H;

X₁₂═X₁₃=“absent”;
a, b, c, and d each represent a single bond; and
e=“absent”.

### IIa:

R1=OR where R═H, C₁-C₄ alkyl, benzyl, 4-hydroxybenzyl, CH₂CH₂CH₂CH₂NH₇, 4-imidazolylmethyl, 3-indolylmethyl, or CH₃SCH₂ group, or an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol, each being carboxymethoxy substituted, or an ester of chrysanthemic acid, cinnamic acid, or retinolic acid;
R2=(C═O)NHCHRₓCOOY where Y═H, Na, K, Ca, Mg, C₁-C₄ alkyl group or NR_{y} where R_{y}═H or a C₁-C₄ alkyl group, and H, benzyl, 4-hydroxybenzyl, CH₂CH₂CH₂CH₂NH₂, 4-imidazolylmethyl or 3-indolylmethyl group or L-aspartate, L-histidine, L-glutamine or L-lysine;

### R3=CH₂═CCH₃;

### X₁₀═X₁₁═H;

X₁₂═X₁₃=“absent”;
a, b, c, and d each represent a single bond; and
e=“absent”.

### IIb:

R1=OR where R═H, C₁-C₄ alkyl, benzyl, 4-hydroxybenzyl, CH₂CH₂CH₂CH₂NH₂, or CH₃SCH₂ group, or an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol, each being carboxymethoxy substituted, or an ester of chrysanthemic acid, cinnamic acid, or retinolic acid;
R2=(C═O)R_{w} where R_{w}═OH, an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol;

### R3=CH₂═CCH₃;

### X₁₀═X₁₁═H;

X₁₂═X₁₃=“absent”;
a, b, c, and d each represent a single bond; and
e=“absent”.

### IJa:

R1=oxo(═O) group;
R2=(C═O)NHCHRₓCOOY where Y═H, Na, K, Ca, Mg, C₁-C₄ alkyl group or NR_{y} where R_{y}═H or a C₁-C₄ alkyl group, and Rₓ=H, C₁-C₄-alkyl-, benzyl, 4-hydroxybenzyl, CH₂CH₂CH₂CH₂NH₂, 4-imidazolylmethyl or 3-indolylmethyl group or 28-aspartate dimethyl ester;

### R3=CH₂CCH₃;

### X₁₀═X₁₁═H;

X₁₂═X₁₃=“absent”;
a, b, c, and d each represent a single bond; and
e=“absent”.

### IJa;

R1=oxo(═O) group;
R2=(C═O)R_{w} where R_{w}═OH, an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol;

### R3=CH₂═CCH₃ or CH₃—CH—CH₃;

### X₁₀═X₁₁═H;

X₁₂═X₁₃=“absent”;
a, b, c, and d each represent a single bond; and
e=“absent”,

### IK:

R1=OH or O—(C═O)R_{b} where R_{b}=C₃-C₈ cyclic or heterocyclic residue, substituted or unsubstituted phenyl or benzyl residue, C₁-C₂₂ linear or branched alkyl or alkenyl group;
R2=CH₂OH or CH₂O—(C═O)R_{f} where R_{f}=C₃-C₈ cyclic or heterocyclic residue, substituted or unsubstituted phenyl or benzyl residue, C₁-C₂₂ linear or branched alkyl or alkenyl group;
R3=(CH₃)₂CR_{z} or CH₃CHCH₂R_{z} where R_{z}=C₆H₅₋ₙ(OH)_{n or C}₆H₅₋ₙ₋ₘ(OH)ₙ—(OCH₃)ₘ and n=0-5, m=0-5, n+m≦5;

### X₁₀═X₁₁═H;

X₁₂═X₁₃=“absent”;
a, b, c, and d each represent a single bond; and
e=“absent”.

### IL:

R1=OH or O—(C═O)R_{b} where R_{b}=C₃-C₈ cyclic or heterocyclic residue, substituted or unsubstituted phenyl or benzyl residue, C₁-C₂₂ linear or branched alkyl or alkenyl group;
R2=CH₂OH or CH₂O—(C═O)R_{f} where R_{f}=C₃-C₈ cyclic, or heterocyclic residue, substituted or unsubstituted phenyl or benzyl residue, C₁-C₂₂ linear or branched alkyl or alkenyl group;
R3=H₂C=CCH₂R_{q} or CH₃CCH₂R_{q} where R_{q}=succinic anhydride, succinic imide or CH(COORₒCH₂COOR_{z} where Rₒ=H, Na, K, Ca, Mg or a C₁-C₂₂ linear or branched alkyl or alkenyl group and R_{z} Na, K, Ca, Mg or a C₁-C₂₂ linear or branched alkyl or alkenyl group;
X₁₀═X₁₃=“absent”;
X₁₂═X₁₃=“absent”;
a, b, c, and d each represent a single bond; and
e=“absent”.

### IM:

R1=H, OR_{z}, O(C═O)R_{b}, NRₐR_{z}, CN, ═NORₐ, CHO, (C═O)OR_{z}, SR_{z}, ═O, ═S where R_{z}=H, C₁-C₆ linear or branched alkyl or alkenyl group, or an aromatic group ZZ shown below, and Rₐ=H, C₁-C₆ linear or branched alkyl or alkenyl group, or an aromatic group ZZ, and R_{b}=H, C₁-C₂₂ linear or branched alkyl or alkenyl group, or an aromatic group ZZ, or R1 corresponds to the partial structure XX shown below;
R2=CH₂OR_{z}, CH₂O(C═O)R_{b}, (C═O)OR_{b}, CH₂NRₐR_{z}, CH₂CN, CN, CH═NORₐ, CH₂CHO, CH₂(C═O)OR_{z}, CH₂SR_{z}, CH═O, CH═S where R_{z}═H, C₁-C₆ linear or branched alkyl or alkenyl group, or an aromatic group ZZ, and Rₐ=H, C₁-C₆ linear or branched alkyl or alkenyl group, or an aromatic group ZZ, and R_{b}═H, C₁-C₂₂ linear or branched alkyl or alkenyl group, or an aromatic group ZZ, or R2 corresponds to the partial structure YY shown below;
R3=CH₂═C—CH₃ or CH₃—CH—CH₃ (isopropyl group);

### X₁₀═X₁₁═H;

X₁₂═X₁₃=“absent”;
a, b, c, and d independently represent a single or a double bond; and
e=“absent”; said partial structures XX and YY, where YY═CH₂XX are selected from the group consisting of

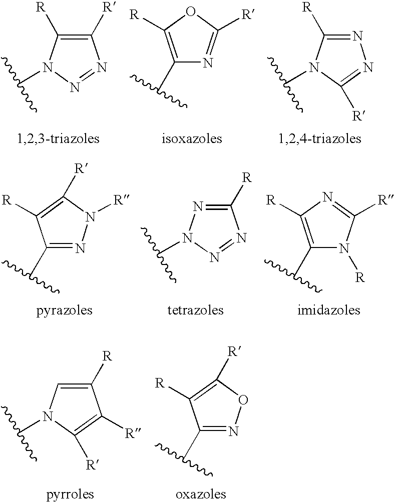

in which structures R, R′, and R″ independently represent H, an aromatic group ZZ, C₁-C₆ linear or branched alkyl or alkenyl group; the aromatic group ZZ being of the form:

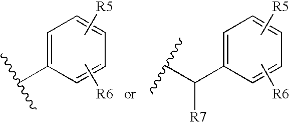

where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen (fluoro, chloro, bromo, iodo), nitro, carboxy, carboxyl, acetyl, R5-R6 forms a cyclic methylenedioxy group, sulfate, cyano, hydroxy or trifluoromethyl group

### IN:

R1=H, OR_{z}, NRₐR_{z}, CN, CHO, (C═O)OR_{z}, O(C═O)R_{b}, O(C═O)NHR_{f}, SR_{z}, ═O or ═S where R_{z}=H, C₁-C₆ linear or branched alkyl or alkenyl group, or an aromatic group ZZ, and Rₐ=H, C₁-C₆ linear or branched alkyl or alkenyl group, or an aromatic group ZZ, and R_{b}═H, C₁-C₂₂ linear or branched alkyl or alkenyl group or an aromatic group ZZ, or R_{b} corresponds to the partial structure YX shown below, and R_{f}=H, C₁-C₆ linear or branched alkyl or alkenyl group, or an aromatic group ZZ or R_{f} corresponds to the partial structure YX shown below; R2=CH₂OR_{z}, (C═O)OR_{b}, CH₂NRₙR_{z}, CH₂CN, CH₂CHO, CH₂(C═O)OR_{z}, CH₂O(C═O)R_{b}, CH₂O(C═O)NHR_{f}, CH₂SR_{z}, CH—O or CH═S where R_{z}H, C₁-C₆ linear or branched alkyl or alkenyl group, or an aromatic group ZZ, and Rₐ=C₁-C₆ linear or branched alkyl or alkenyl group, or an aromatic group ZZ, and R_{b}=H, C₁-C₂₂ linear or branched alkyl or alkenyl group or an aromatic group ZZ, or R_{b} corresponds to the partial structure YX shown below, and R_{f}=H, C₁-C₆ linear or branched alkyl or alkenyl group, or an aromatic group ZZ or R_{f} corresponds to the partial structure YX shown below;

### R3=CH₂═C—CH₃ or CH₃—CH—CH₃;

### X₁₀═X₁₁═H;

X₁₂═X₁₃=“absent”;
a, b, c, and d independently represent a single or a double bond; and
e=“absent”; and
said aromatic group ZZ being of the form:

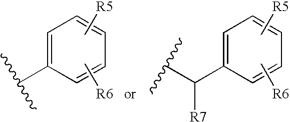

where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen (fluoro, chloro, bromo, iodo), nitro, carboxy, carboxyl, acetyl, R5-R6 forms a cyclic methylenedioxy group, sulfate, cyano, hydroxy or trifluoromethyl group; and the partial structure R_{f} or R_{b} is of the form YX:

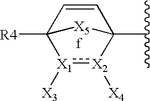

where R4=H or a C₁-C₂₀ linear or branched alkyl or alkenyl group or an aromatic group ZZ;
X₅=“absent”, C, O, N, or S;
X₁-X₂ forms a cyclic partial structure of the form:
X₁—(X₃═X₆)—X₇—(X₄=X₈)—X₂ where

### X₁=X₂=C or N;

### X₃=X₄═C;

X₆=X₈═O, S or “absent”;
X₇=C, O, S, or N—X₉ where X₉=H, C₁-C₆ linear or branched alkyl or alkenyl group, or an aromatic group ZZ; and
f=a single or a double bond.

### IO:

R1=H, OR_{z}, NRₐR_{z}, CN, CHO, (C═O)OR_{z}, O(C═O)R_{b}, O(C═O)NHR_{f}, SR_{z}, ═O or ═S where R_{z}═H, C₁-C₆ linear or branched alkyl or alkenyl group or an aromatic group ZZ, and Rₐ=H, C₁-C₆ linear or branched alkyl or alkenyl group, or an aromatic group ZZ, and R_{b}═H, C₁-C₂₂ linear or branched alkyl or alkenyl group, or an aromatic group ZZ, or R_{b} corresponds to the partial structure YX shown below, and R_{f}=H, C₁-C₆ linear or branched alkyl or alkenyl group, or an aromatic group ZZ or R_{f} corresponds to the partial structure YX′ shown below;
R2=CH₂OR_{z}, (C═O)OR_{b}, CH₂NR_{z}R_{z}, CH₂CN, CH₂CHO, CH₂(C═O)OR_{z}, CH₂O(C═O)R_{b}, CH₂O(C═O)NHR_{f}, CH₂SR_{z}, CH═O or CH═S where R_{z}═H, C₁-C₆ linear or branched alkyl or alkenyl group, or an aromatic group ZZ, and H, C₁-C₆ linear or branched alkyl or alkenyl group, or an aromatic group ZZ, and R_{b}=H, C₁-C₂₂ linear or branched alkyl or alkenyl group, or an aromatic group ZZ, or R_{b} corresponds to the partial structure YX shown below and R_{f}=H, C₁-C₆ linear or branched alkyl or alkenyl group, or an aromatic group ZZ or R_{f} corresponds to the partial structure YX shown below;

### R3=CH₂—C—CH₃ or CH₃—CH—CH₃;

### X₁₀═X₁₁═H

X₁₂═X₁₃=“absent”;
a, b, c, and d independently represent a single or a double bond; and
e=“absent”;
said aromatic group ZZ being of the form:

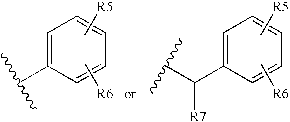

where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen (fluoro, chloro, bromo, iodo), nitro, carboxy, carboxyl, acetyl, R5-R6 forms a cyclic methylenedioxy group, sulfate, cyano, hydroxy or trifluoromethyl group; and
the partial structure R_{f} or R_{b} is of the form YX:

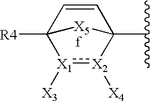

where R4=H or a C₁-C₂₀ linear or branched alkyl or alkenyl group, or an aromatic group ZZ;
X₅=“absent”, C, O, N, or S;

### X₁=X₂=C or N; and

X₃=X₄=R₅, (C═O)OR_{g} or (C═O)NHR_{g} where R_{g}═H, C₁-C₆ linear or branched alkyl or alkenyl group; and
f=a single or a double bond

### IP:

R1=H, OR, NRₐR_{z}, CN, CHO, (C═O)OR_{z}, O(C═O)R_{b}, O(C═O)NHR_{z}, SR_{z}, ═O or ═S, where R_{z}=H, C₁-C₆ linear or branched alkyl or alkenyl group, or an aromatic group ZZ, and Rₐ=H, C₁-C₆ linear or branched alkyl or alkenyl group, or an aromatic group ZZ, and R_{b}═H, C₁-C₂₂ linear or branched alkyl or alkenyl group, or an aromatic group ZZ;
R2=CH₂OR_{z}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{z}, CH₂SR_{z}, CH—O or CH=S, where R_{z}=H, C₁-C₆ linear or branched alkyl or amyl group or an aromatic group ZZ, and Rₐ, ═H, C₁-C₆ linear or branched alkyl or alkenyl group or an aromatic group ZZ, and R_{b}=H, C₁-C₂₂ linear or branched alkyl or alkenyl group or an aromatic group ZZ;

### R3=CH₂═C—CH₃ or CH₃—CH—CH₃; and

said aromatic group ZZ being of the form:

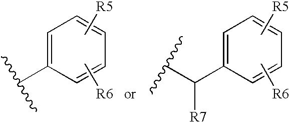

where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen (fluoro, chloro, bromo, iodo), nitro, carboxy, carboxyl, acetyl, R5-R6 forms a cyclic methylenedioxy group, sulfate, cyano, hydroxy or trifluoromethyl group; and at X₁₀-X₁₁, a cyclic or heterooyclic partial structure having the form X₁₀—(X₁₂═X₁₄)—X₁₅—(X₁₃=X₁₆)—X₁₁ may be present where X₁₀═X₁₁=C or N;

### X₁₂═X₁₃—C;

X₁₄=X₁₆=O, S or “absent”;
X₁₅=C, O, S, or N—X₁₇ where X₁₇=H, a C₁-C₆ linear or branched alkyl or alkenyl group, or an aromatic group ZZ; and a, b, c, d and a independently represent double or single bonds

### IQ:

R1=H, OR_{z}, CN, CHO, (C═O)OR_{z}, O(C═O)R_{b}, O(C═O)NHR_{z}, SR_{z}, ═O or ═S where R_{z}═H, C₁-C₆ linear or branched alkyl or alkenyl group, or an aromate group ZZ, and Rₐ=H, C₁-C₆ linear or branched alkyl or alkenyl group, or an aromatic group ZZ, and R_{b}═H, C₁-C₂₂, linear or branched alkyl or alkenyl group, or an aromatic group ZZ;
R2=CH₂OR_{z}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{z}, CH₂SR_{z}, CH═O or CH=S where R_{z}H, C₁-C₆ linear or branched alkyl or alkenyl group, or an aromatic group ZZ, and R_{z}=H, C₁-C₆ linear or branched alkyl or alkenyl group, or an aromatic group ZZ, and R_{b}=H, C₁-C₂₂ linear or branched alkyl or alkenyl group or an aromatic group ZZ;

### R3=CH₂=C—CH₃ or CH₃—CH—CH₃; and

said aromatic group ZZ being of the form:

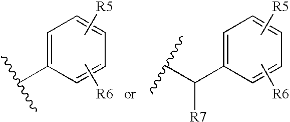

where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen (fluoro, chloro, bromo, iodo), nitro, carboxy, carboxyl, acetyl, R5-R6 forms a cyclic methylenedioxy group, sulfate, cyano, hydroxy or trifluoromethyl group; and
at X₁₀-X₁₁, a novel cyclic or heterocyclic partial structure may be present where X₁₀═X₁₁=C or N;
X₁₂═X₁₃=R, (C═O)OR or (C═O)NHR where R═H or a C₁-C₆ linear or branched alkyl or alkenyl group, or an aromatic group ZZ; and
a, b, c, d and e independently represent double or single bonds.

Preferable compounds derived from betulin for the preparation of a drug against leishmaniasis include compounds selected from the group consisting of betulonic alcohol 28-acetate, betulonic acid 28-methylester, betulin 3,28-dioxime, betulin 28-oxime, betulonic alcohol, betulin 3-acetoxime-28-nitrile, betulin 28-acetic acid methylester, 20,29-dihydrobetulonic acid, betulonic acid, 28-aspartateamide dimethylester of betulonic acid, betulin 28-N-acetylanthranilic acid ester, Diels-Alder adduct of 3β,28-diacetoxylupa-12,18-diene and urazole, Diels-Alder adduct of 3β,28-diacetoxylupa-12,18-diene and 4-methylurazole, Diels-Alder adduct of 3β,28-diacetoxylupa-12,18-diene and 4-phenylurazole, Diels-Alder adduct of 3β,28-diacetoxylupa-12,18-diene and p-fluoro-4-phenylurazole, Dials-Alder adduct of 3β,28-diacetoxylupa-12,18-diene and m-methoxy-4-phenylurazole, Diels-Alder adduct of 3β,28-diacetoxylupa-12,18-diene and m-acetoxy-4-phenylurazole, Dials-Alder adduct of 3β,28-diacetoxylupa-12,18-diene and 1-naphthylurazole, and Dials-Alder adduct of 3β,28-diacetoxylupa-12,18-diene and 1,3-dioxol-5-ylurazole.

Particularly preferable compounds for the preparation of a drug against leishmaniasis include compounds selected from the group consisting of betulin 3,28-dioxime, betulin 28-oxime, betulonic alcohol, betulin 3-acetoxime-28-nitrile, betulin 28-acetic acid methylester, 20,29-dihydrobetulonic acid, betulonic acid, 28-aspartateamide dimethylester of betulonic acid, betulin 28-N-acetylanthranilic acid ester, Diels-Alder adduct of 3β,28-diacetoxylupa-12,18-diene and urazole, Diels-Alder adduct of 3β,28-diacetoxylupa-12,18-diene and 4-methylurazole, Diels-Alder adduct of 3β,28-diacetoxylupa-12,18-diene and 1-naphthylurazole, and Diels-Alder adduct of 3β,28-diacetoxylupa-12,18-diene and 1,3-dioxol-5-ylurazole.

Novel compounds derived from betulin, useful as agents against leishmaniasis according to the invention include betulin derivatives of the general formula I and pharmaceutically acceptable salts thereof, where in formula I

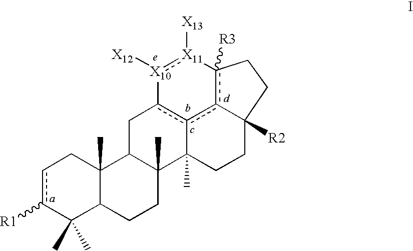

to R1=H, —ORₐ, —O(C═O)R_{b}, —NRₙR_{z}, —CN, —CHO, —(C═O)ORₐ, —SRₐ, —O(C═O)NHRₐ, ═O or ═S where Rₐ, R_{b} and R_{z} independently represent H, C₁-C₂₂ aliphatic, unbranched or branched, saturated or unsaturated hydrocarbon residue, with the proviso that X₁₀═X₁₁ is not H; C₃-C₈ cyclic or heterocyclic residue; substituted or unsubstituted phenyl or benzyl residue; amine, amide or amino acid; substituted or unsubstituted 1,2,3-triazol, 1,2,4-triazol, tetrazol, pyrrole, isoxazol, pyrazol, imidazol, or oxazol; a carboxymethyl, carboxymethylester or carboxymethylamide derivative or a salt thereof;
R2=—CH₂ORₐ, —CH₂O(C═O)R_{b}, —(C═O)OR_{b}, —CH₂NRₐR_{z}, —CH₂CN, —CH₂CHO, —CH₂(C═O)ORₙ, —CH₂SRₐ, —CH₂O(C═O)NHRₐ, —CH═S or —CH═S where Rₐ, R_{b} and R_{z} independently represent H, C₁-C₂₂ aliphatic, unbranched or branched, saturated or unsaturated hydrocarbon residue, with the proviso that X₁₀═X₁₁ is not H; C₃-C₈ cyclic or heterocyclic residue; substituted or unsubstituted phenyl or benzyl residue; unsubstituted or substituted 1,2,3-triazol, 1,2,4-triazol, tetrazol, pyrrole, isoxazol, pyrazol, imidazol, or oxazol; a carboxymethyl, carboxymethylester or carboxymethylamide derivative or a salt thereof;
R3=isopropenyl, isopropyl, isopropylphenyl, isopropylhydroxyphenyl, or isopropylsuccinic acid derivative or a salt thereof;

### X₁₀═X₁₁═H, C or N;

X₁₂═X₁₃=“absent”; (C═O)OR, (C═O)NHR where R═H or a C₁-C₆ linear or to branched alkyl or alkenyl group or substituted or unsubstituted phenyl or benzyl residue or X₁₂-X₁₃ forms a cyclic partial structure of the form —(X₁₂═X₁₄)—X₁₅-(X₁₃=X₁₆)— where X₁₂═X₁₃=C, X₁₄=X₁₆=“absent”, O or S, X₁₅═C, O, S or N—X₁₇ where X₁₇=H, C₁-C₆ linear or branched alkyl or alkenyl group, substituted or unsubstituted phenyl or benzyl residue;
a, b, c and d independently represent o, double or single bond; and
e=“absent” or represents a double or single bond.

In case X₁₀═X₁₁═H, X₁₂═X₁₃=“absent”, a, b, c and d each represent a single bond and e=“absent”, then R1, and R_{R}, R_{b} and R_{z} present in R2 independently represent a C₁₁-C₂₂ aliphatic, unbranched or branched, saturated or unsaturated hydrocarbon residue with the proviso that at the same time R1 represents ═O (oxo) or ═S; C₃-C₈ cyclic or heterocyclic residue, substituted or unsubstituted phenyl residue, substituted or unsubstituted 1,2,3-triazol, 1,2,4-triazol, tetrazol, pyrrole, isoxazol, pyrazol, imidazol, or oxazol, a carboxymethyl, carboxymethylester or carboxymethylamide derivative or a salt thereof.

In a preferable embodiment of the invention, R1=OH, R2=CH₂O(C═O)R_{f} or —CH₂ORₐ(C═O)OR_{f} where R_{f}=C₃-C₈ cyclic or heterocyclic residue, substituted or unsubstituted phenyl residue, Rₐ=C₁-C₂₂ linear or branched alkylene or alkenyl group, R3=CH₂═CCH₃, X₁₀═X₁₁═H, X₁₂═X₁₃=absent; a, b, c, and d each represent a single bond, and e=“absent”.

In another preferable embodiment of the invention, R1=OH, R2=CH₂O(C═O)(CHR_{g})CH₂COOY where R_{g}=C₄-C₂₂ linear or branched alkyl or alkenyl group, Y═H, Na, K, Ca, Mg, C₁-C₄-alkyl group, or NRₕ where Rₕ=H or C₁-C₄-alkyl group, R3=CH₂=CCH₃, X₁₀═X₁₁═H, X₁₂═X₁₃=absent, a, b, c, and d each represent a single bond, and e=absent,

In still another preferable embodiment of the invention, R1=OH, R2=CH₂OR; where R₁=an ester of ornithine, N-acetylanthranilic acid or trimethylglycine; R3=CH₂═CCH₃, X₁₀═X₁₁═H, X₁₂═X₁₃=absent, a, b, c, and d each represent a single bond, and e=absent.

In still another preferable embodiment of the invention, R1=OH, R2=CH₂O(C═O)CHRⱼ(NHZ) or —CH₂ORₐ(C═O)NHRⱼ where Rₐ=C₁-C₂₂ linear or branched alkylene or alkenyl group; Rⱼ=CH₂CH₂CH₂CH₂NH₂, 4-imidazolylmethyl or 3-indolylmethyl group, and Z=H, Rₖ, (C═O)Rₖ or COORₖ where Rₖ=C₁-C₂₂ branched or unbranched alkyl or alkenyl group, or a phenyl, benzyl or 4-hydroxybenzyl group, R3=CH₂=CCH₃, X₁₀═X₁₁═H, X₁₂═X₁₃ absent, a, b, c, and d each represent a single bond, and e=absent.

In still another preferable embodiment of the invention, R1=OH, R2=CH₂ORₙ where Rₙ=an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol, each being carboxymethoxy substituted, or an ester of chrysanthemic acid, cinnamic acid, or retinolic acid, R3=CH₂=CCH₃, X₁₀═X₁₁H, X₁₂═X₁₃=absent, a, b, c, and d each represent a single bond; and e=absent.

In still another preferable embodiment of the invention, R1=O(C═O)Rₘ or —ORₐ(C═O)ORₘ where Rₘ=C₃-C₈ cyclic or heterocyclic residue, substituted or unsubstituted phenyl or benzyl residue, Rₐ=C₁-C₂₂ linear or branched alkylene or alkenyl group; R2=CH₂O(C═O)Rₒ or CH₂ORₐ(C═O)Rₒ where Rₒ=C₃-C₈ cyclic or heterocyclic residue, substituted or unsubstituted phenyl or benzyl residue, Rₐ=C₁-C₂₂ linear or branched alkylene or alkenyl group, R3=CH₂=CCH₃, X₁₀═X₁₁=H, X₁₂═X₁₃=absent, a, b, c, and d each represent a single bond, and e=absent.

In still preferable embodiment of the invention, R1=O(C═O)(CHR_{c})CH₂COOY where R_{c}=C₄-C₂₂ linear or branched alkyl or alkenyl group, Y=H, Na, K, Ca, Mg, C₁-C₄ alkyl group or NRₕ where Rₕ=H or a C₁-C₄ alkyl group; R2=CH₂O(C═O)(CHR_{d})CH₂COOY where R_{d}=C₄-C₂₂ linear or branched alkyl or alkenyl group, Y=H, Na, K, Ca, Mg, C₁-C₄ alkyl group or NRₖ where Rₖ=H or a C₁-C₄ alkyl group, R3=CH₂═CCH₃, X₁₀═X₁₁=X₁₂═X₁₃=absent, a, b, c, and d each represent a single bond, and e=“absent”.

In still another preferable embodiment of the invention, R1=ORᵣ where Rᵣ=an ester of ornithine, N-acetylanthranilic acid or trimethylglycine, R2=CH₂ORₚ where Rₚ=an erster of ornithine, N-acetylanthranilic acid or trimethylglycine, R3=CH₂=CCH₃, X₁₀═X₁₁═H, X₁₂=X₁₃=absent, a, b, c, and d each represent a single bond, e=absent.

In still another preferable embodiment of the invention, R1=O(C═O)CHRₛ(NHZ) or ORₐ(C═O)NHRₛ where Rₐ=C₁-C₂₂ linear or branched alkylene group; Rₛ=CH₂CH₂CH₂CH₂NH₂, 4-imidazolylmethyl or 3-indolylmethyl group, Z=H, Rₖ, (C═O)Rₖ or COORₖ where Rₖ=C₁-C₂₂ branched or unbranched alkyl or alkenyl group, or a phenyl, benzyl or 4-hydroxybenzyl group, R2=CH₂O(C═O)CHRₓ(NHZ) or —CH₂ORₐ(C═O)NHRₓ where Rₐ=C₁-C₂₂ linear or branched alkylene group; Rₓ=CH₂CH₂CH₂CH₂NH₂, 4-imidazolylmethyl or 3-indolylmethyl group, Z=H, R_{y}, (C═O)R_{y} or COOR_{y} where R_{y}=C₁-C₂₂ branched or unbranched alkyl or alkenyl group, or a phenyl, benzyl or 4-hydroxybenzyl group, R3=CH₂=CCH₃, X₁₀═X₁₁═H, X₁₂═X₁₃=absent, a, b, c, and d each represent a single bond, and e=absent.

In still another preferable embodiment of the invention, R1=ORᵥ where Rᵥ=an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol, each being carboxymethoxy substituted, or an ester of chrysanthemic acid, cinnamic acid, or retinolic acid, R2=CH₂ORᵤ where Rᵤ=an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol, each being carboxymethoxy substituted, or an ester of chrysanthemic acid, cinnamic acid, or retinolic acid, R3=CH₂=CCH₃, X₁₀═X₁₁═H, X₁₂═X₁₃=absent, a, b, c, and d each represent a single bond, and e=absent.

In still another preferable embodiment of the invention, R1=OH, R2=(C═O)NHCHRₓCOOY where Y=H, Na, K, Ca, Mg, C₁-C₄ alkyl group or NR_{y} where R_{y}═H or a C₁-C₄ alkyl group, and Rₓ═CH₂CH₂CH₂CH₂NH₂, 4-imidazolylmethyl or 3-indolylmethyl group, R3=CH₂═CCH₃, X₁₀═X₁₁═H, X₁₂=X₁₃=absent, a, b, c, and d each represent a single bond, and e=absent.

In still another preferable embodiment of the invention, R1=OH, R2=(C═O)R_{w} where R_{w}=an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol, R3=CH₂=CCH₃, X₁₀═X₁₁═H, X₁₂═X₁₃=absent, a, b, c, and d each represent a single bond, and e=absent.

In still another preferable embodiment of the invention, R1=OR where R═H, C₁-C₄ alkyl, benzyl, 4-hydroxybenzyl, —CH₂CH₂CH₂CH₂NH₂, 4-imidazolylmethyl, 3-indolylmethyl or CH₃SCH₂ group, or an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol, each being carboxymethoxy substituted, or an ester of chrysanthemic acid, cinnamic acid, or retinolic acid, R2=(C═O)NHCHRₓCOOY where Y═H, Na, K, Ca, Mg, C₁-C₄ alkyl group or NR_{y} where R_{y}=H or a C₁-C₄ alkyl group, and Rₓ=—CH₂CH₂CH₂CH₂NH₂, 4-imidazolylmethyl or 3-indolylmethyl group, R3=CH₂=CCH₃, X₁₀═X₁₁═H, X₁₂═X₁₃=absent, a, b, c, and cl each represent a single bond, and e=absent.

In still another preferable embodiment of the invention, R1=OR where R═H, C₁-C₄ alkyl, benzyl, 4-hydroxybenzyl, —CH₂CH₂CH₂CH₂NH₂ or CH₃SCH₂ group, or an ester of verbenol, terpineol, thymol, carvacrol, menthol, cinnamic alcohol, curcumin, eugenol, borneol, isoborneol, longifolol, isolongifolol, globulol, sedrol, or episedrol, each being carboxymethoxy substituted, or an ester of chrysanthemic acid, cinnamic acid, or retinolic acid, R2=(C═O)R_{w} where R_{w}=an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol, R3=CH₂═CCH₃, X₁₀═X₁₁═H, X₁₂═X₁₃=absent, a, b, c, and d each represent a single bond, and e=absent.

In still another preferable embodiment of the invention, R1=oxo group (═O, R2=(C═O)NHCHRₓCOOY where Y=H, Na, K, Ca, Mg, C₃-C₄ alkyl group or NR_{y} where R_{y}═H or a C₁-C₄ alkyl group, and Rₓ═CH₂CH₂CH₂CH₂NH₂, 4-imidazolylmethyl or 3-indolylmethyl group, or L-aspartate, L-histidine, L-glutamine, L-lysine, or 28-aspartate dimethylester, R3=CH₂═CCH₃, X₁₀═X₁₁=H, X₁₂═X₁₃=absent, a, b, c, and d each represent a single bond, and e=absent.

In still another preferable embodiment of the invention, R1=oxo group, R2=(C═O)R_{w} where R_{w}=an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol, R3=CH₂═CCH₃, X₁₀═X₁₁═H, X₁₂═X₁₃=absent, a, b, c, and d each represent a single bond, and e=absent,

In still another preferable embodiment of the invention, R1=OH or O—(C═O)R_{b} where R_{b}═C₃-C₈ cyclic or heterocyclic residue, substituted or unsubstituted phenyl or benzyl residue, C₁-C₂₂ linear or branched alkyl or alkenyl group, R2=CH₂OH or CH₂O—(C═O)R_{f} where R_{f}═C₃-C₈ cyclic or heterocyclic residue, substituted or unsubstituted phenyl or benzyl residue; C₁-C₂₂ linear or branched alkyl or alkenyl group, R3=(CH₃)₂CR_{z} or CH₃CHCH₂R_{z} where R_{z}═C₆H₅₋ₙ(OH)ₙ or C₆H₅₋ₙ₋ₘ(OH)ₙ(OCH₃)ₘ and m=0-5, n=0-5, n+m≦5, X₁₀═X₁₁═H, X₁₂═X₁₃=absent, a, b, c, and d each represent a single bond, and e=absent.

In still another preferable embodiment of the invention, R1=OH or O—(C═O)R_{b} where R_{b}═C₃-Cₛ cyclic or heterocyclic residue, substituted or unsubstituted phenyl or benzyl residue, C₁-C₂₂ linear or branched alkyl or alkenyl group, R2=CH₂OH or CH₂O—(C═O)R_{f} where R_{f}═C₃-C₈ cyclic or heterocyclic residue, substituted or un substituted phenyl or benzyl residue, C₁-C₂₂ linear or branched alkyl or alkenyl group, R3=H₂C═CCH₂R_{q} or CH₃CCH₂R_{q} where R_{q}=succinic anhydride, succinic imide or CH(COORₒCH₂COOR_{z} where Rₒ═H, Na, K, Ca, Mg or a C₁-C₂₂ linear or branched alkyl or alkenyl group and R_{z}═H, Na, K, Ca, Mg or a C₁-C₂₂ linear or branched alkyl or alkenyl group, X₁₀═X₁₁═H, X₁₂═X₁₃=absent, a, b, c, and d each represent a single bond, and e=absent.

In still another preferable embodiment of the invention, R1=H, OR_{z}, O(C═O)R_{b}, NRₐR_{z}, CN, ═NOR_{R}, CHO, (C═O)OR_{z}, SR_{z}, ═O, ═S where R_{z}═H, C₁₋₅ linear or branched alkyl or alkenyl group, or an aromatic group ZZ shown below, and Rₐ═H, C₁-C₆ linear or branched alkyl or alkenyl group, or an aromatic group ZZ, and R_{b}═H, C₁-C₂₂ linear or branched alkyl or alkenyl group, or an aromatic group ZZ, or R1 corresponds to the partial structure XX shown below; R2=CH₂OR_{z}, CH₂O(C═O)R_{b}, (C═O)OR_{b}, CH₂NRₐR_{z}, CH₂CN, CN, CH═NORₐ, CH₂CHO, CH₂(C═O)OR_{z}, CH₂SR_{z}, CH═O, CH═S where R_{z}═H, C₁-C₆ linear or branched alkyl or alkenyl group, or an aromatic group ZZ, and Rₐ═C₁-C₆ linear or branched alkyl or alkenyl group, or an aromatic group ZZ, and R_{b}═H, C₁-C₂₂ linear or branched alkyl or alkenyl group, or an aromatic group ZZ, or R2 corresponds to the partial structure YY shown below, with the proviso that R1 or R2 comprises the group ZZ or XX; R3=CH₂═C—CH₃ or CH₃—CH—CH₃ (isopropyl group); X₁₀═X₁₁═H; X₁₂═X₁₃=absent, a, b, c, and d independently represent a single or a double bond; and e=absent; said partial structures XX and YY where YY CH₂XX being selected from the group consisting of:

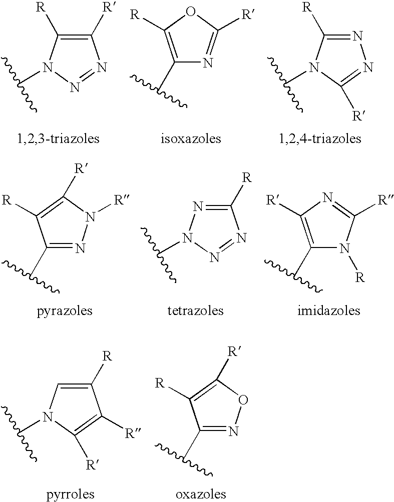

in which structures R, R′, and R″ independently represent H, an aromatic group ZZ, C₁-C₆ linear or branched alkyl or alkenyl group; and the aromatic group ZZ being of the form:

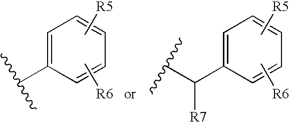

where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen (fluoro, chloro, bromo, iodo), nitro, carboxy, carboxyl, acetyl, R5-R6 forms a cyclic methylenedioxy group, sulfate, cyano, hydroxy or trifluoromethyl group.

In still another preferable embodiment of the invention, R1=H, OR_{z}, NRₐR_{z}, CN, CHO, (C═O)OR_{z}, O(C═O)R_{b}, O(C═O)NHR_{f}, SR_{z}, ═O or ═S where R_{z}═H, C₁-C₆ linear or branched alkyl or alkenyl group or an aromatic group ZZ, and Rₐ═H, C₁-C₆ linear or branched alkyl or alkenyl group or an aromatic group ZZ, and R_{b}═H, C₁-C₂₂ linear or branched alkyl or alkenyl group or an aromatic group ZZ, or R_{b} corresponds to the partial structure YX shown below, and Rₜ═H, C₁-C₆ linear or branched alkyl or alkenyl group or an aromatic group ZZ or R_{f} corresponds to the partial structure YX shown below; R2=CH₂O R_{z}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{f}, CH₂SR_{z}, to CH═O tai CH═S where R_{z}═H, C₁-C₆ linear or branched alkyl or alkenyl group or an aromatic group ZZ, and Rₐ═H, C₁-C₆ linear or branched alkyl or alkenyl group, or an aromatic group ZZ, and R_{b}═H, C₁-C₂₂ linear or branched alkyl or alkenyl group or an aromatic group ZZ, or R_{b} corresponds to the partial structure YX shown below, and R_{f}═H, C₁-C₆ linear or branched alkyl or alkenyl group or an aromatic group ZZ or R_{f} corresponds to the partial structure YX shown below, with the proviso that R1 or R2 comprises the group ZZ or YX; R3=CH₂═C—CH₃ or CH₃—CH—CH₃; X₁₀═X₁₁═H, X₁₂═X₁₃=“absent”; a, b, c, and d independently represent a single or a double bond; and e=“absent”; the aromatic group ZZ being of the form:

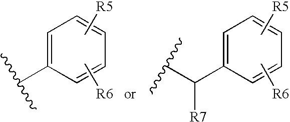

where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen (fluoro, chloro, bromo, lode), nitro, carboxy, carboxyl, acetyl, R5-R6 forms a cyclic methylenedioxy group, sulfate, cyano, hydroxy or trifluoromethyl group; and the partial structure R_{f} or R_{b} is of the form YX;

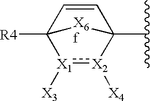

where R4=H or a C₁-C₂₀ linear or branched alkyl or alkenyl group or an aromatic group ZZ; X₅=“absent”, C, O, N, or S; X₁-X₂ forms a cyclic partial structure of the form: X₁—(X₃═X₆)—X₇—(X₄=X₈)—X₂ where X₁═X₂═C or N; X₃═X₄═C; X₆═X₈═O, S or “absent”; X₇═C, O, S, or N—X₉ where X₉═H, C₁-C₆ linear or branched alkyl or alkenyl group, or an aromatic group ZZ; and f=a single or a double bond.

In still another preferable embodiment of the invention, R1=H, OR_{z}, NRₐR_{z}, CN, CHO, (C═O)OR_{z}, O(C═O)R_{b}, O(C═O)NHR_{f}, SR_{z}, ═O or ═S where R_{z}═H, C₁-C₆ linear or branched alkyl or alkenyl group or an aromatic group ZZ, and Rₐ═H, C₁-C₆ linear or branched alkyl or alkenyl group or an aromatic group ZZ, and R_{b}═H, C₁-C₂₂ linear or branched alkyl or alkenyl group, or an aromatic group ZZ, or R_{b} corresponds to the partial structure YX shown below and R_{f}═H, C₁-C₆ linear or branched alkyl or alkenyl group or an aromatic group ZZ or R_{f} corresponds to the partial structure YX shown below; R2=CH₂OR_{z}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{f}, CH₂SR_{Z}, CH—O, CR═S where R_{z}H, C₁-C₆ linear or branched alkyl or alkenyl group, or an aromatic group ZZ, and RₐH, C₁-C₆ linear or branched alkyl or alkenyl group, or an aromatic group ZZ, and R_{b}═H, C₁-C₂₂ linear or branched alkyl or alkenyl group, or an aromatic group ZZ, or R_{b} corresponds to the partial structure YX shown below, and R_{f}=H, linear or branched alkyl or alkenyl group or an aromatic group ZZ or R_{f} corresponds to the partial structure YX shown below; with the proviso that R1 or R2 comprises the group ZZ or YX; R3=CH₂═C—CH₃ or CH₃—CH—CH₃; X₁₀═X₁₁═H; X₁₂═X₁₃=“absent”; a, b, c, and d independently represent a single or a double bond; e=“absent”; said aromatic group ZZ being of the form:

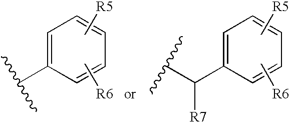

where R5, R6 and/or R7 may be H, a C₁-C₆ linear or branched alkyl or alkenyl group, C₁-C₆ linear or branched alkyl or alkenyl ether, R5-R6 forms a cyclic C₂-C₆ alkyl or alkenyl group, halogen (fluoro, chloro, bromo, iodo), nitro, carboxy, carboxyl, acetyl, R₅-R₆ forms a cyclic methylenedioxy group, sulfate, cyano, hydroxy or trifluoromethyl group; and the partial structure R_{f} or R_{b} is of the form YX:

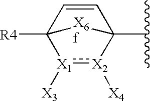

where R4=H or a C₁-C₂₀ linear or branched alkyl or alkenyl group, or an aromatic group ZZ; X₅=“absent”, C, O, N, or S; X₁═X₂═C or N; and X₃═X₄═R_{g}, (C═O)OR_{g} or (C═O)NHR_{g} where R_{g}═H, C₁-C₆ linear or branched alkyl or alkenyl group; and f=a single or a double bond.

In still another preferable embodiment of the invention, R1=H, OR, NRₐR_{z}, CN, CHO, (C═O)OR_{z}, O(C═O)R_{b}, O(C═O)NHR_{z}, SR_{z}, ═O or ═S where R_{z}═H, C₁-C₆ linear or branched alkyl or alkenyl group, or an aromatic group ZZ, and Rₐ═H, C₁-C₆ linear or branched alkyl or alkenyl group or an aromatic group ZZ, and R_{b}═H, C₁-C₂₂ linear or branched alkyl or alkenyl group or an aromatic group ZZ; R2=CH₂OR_{z}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{z}, CH₂SR_{z}, CH═O, CH═S where R_{z}═H, C₁-C₆ linear or branched alkyl or alkenyl group, or an aromatic group ZZ, and Rₐ═H, C₁-C₆ linear or branched alkyl or alkenyl group, or an aromatic group ZZ, and Rₕ═H, C₁-C₂₂ linear or branched alkyl or alkenyl group, or an aromatic group ZZ; with the proviso that R1 or R2 comprises the group ZZ; R3=CH₂═C—CH₃ or CH₃—CH—CH₃; and ZZ being of the form:

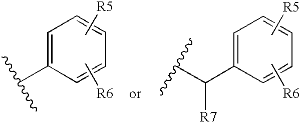

where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen (fluoro, chloro, bromo, iodo), nitro, carboxy, carboxyl, acetyl, R5-R6 forms a cyclic methylenedioxy group, sulfate, cyano, hydroxy or trifluoromethyl group; at X₁₀-X₁₁, a cyclic or heterocyclic partial structure having the form X₁₀—(X₁₂=X₁₄)—X₁₅—(X₁₃═X₁₆)—X₁₁ may be present where X₁₀═X₁₁═C or N; X₁₂═X₁₃═C; X₁₄═X₁₆═O, S or “absent”; X₁₅═C, O, S or N—X₁₇ where X₁₇═H, a C₁-C₆ linear or branched alkyl or alkenyl group, or an aromatic group ZZ; and a, b, c, d and a independently represent double or single bonds.

In still another preferable embodiment of the invention, R1=H, OR_{z}, NRₐR_{z}, CN, CHO, (C═O)OR_{z}, O(C═O)R_{b}, O(C═O)NHR_{z}, SR_{z}, ═O or ═S where R_{z}═H, C₁-C₆ linear or branched alkyl or alkenyl group or an aromatic group ZZ, and Rₐ═H, C₁-C₆ linear or branched alkyl or alkenyl group or an aromatic group ZZ, and R_{b}═H, C₁-C₂₂ linear or branched alkyl or alkenyl group or an aromatic group ZZ; R2═CH₂OR_{z}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{z}, CH₂SR_{z}, CH—O, CH═S where R_{z}═H, C₁-C₆ linear or branched alkyl or alkenyl group, or an aromatic group ZZ, and Rₐ═H, C₁-C₆ linear or branched alkyl or alkenyl group or an aromatic group ZZ, and R_{b}═H, C₁-C₂₂ linear or branched alkyl or alkenyl group, or an aromatic group ZZ; with the proviso that R1 or R2 comprises the group ZZ; R3=CH₂═C—CH₃ or CH₃—CH—CH₃; and said aromatic group ZZ being of the form;

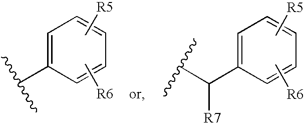

where R5, R6 and/or R7 may be H, a C₁-C₆ linear or branched alkyl or alkenyl group, a C₁-C₆ linear or brandied alkyl or alkenyl ether, R5-R6 forms a cyclic C₂-C₆ alkyl or alkenyl group, halogen (fluoro, chloro, bromo, iodo), nitro, carboxy, carboxyl, acetyl, R5-R6 forms a cyclic methylenedioxy group, sulfate, cyano, hydroxy or trifluoromethyl group; and at X₁₀-X₁₁, a novel cyclic or heterocyclic partial structure may be present where X₁₀═X₁₁═C or N; X₁₂═X₁₃═R, (C═O)OR or (C═O)NHR where R═H or a C₁-C₆ linear or branched alkyl or alkenyl group, or an aromatic group ZZ; and a, b, c, d and e independently represent double or single bonds.

Novel betulin derivatives of the invention include amino acid, anthranilic acid, chrysanthemic acid, ornithine acid, cinnamic acid, retinolic acid, and trimethyl glycine, alpha-terpineol, verbenol, thymol, carvacrol, menthol, cinnamic alcohol, curcumin, eugenol, borneol, isoborneol, longifolol, isolongifolol, globulol, epiglobulol, sedrol, and episedrol derivatives of betulin, betulonic acid or betulinic acid, betulin 3-acetoxime-28-nitrile, betulin 28-acetic acid methylester, betulin 28-N-acetylanthranilic acid ester, Dials-Alder adduct of 3β,28-diacetoxylupa-12,18-diene and urazole, Diels-Alder adduct of 3β,28-diacetoxylupa-12,18-diene and 4-methylurazole, Diels-Alder adduct of 3β,28-diacetoxylupa-12,18-diene and p-fluoro-4-phenylurazole, Diels-Alder adduct of 3β,28-diacetoxylupa-12,18-diene and m-methoxy-4-phenylurazole, Dials-Alder adduct of 3β,28-diacetoxylupa-12,18-diene and 1-naphthylurazole, and Diels-Alder adduct of 3β,28-diacetoxylupa-12,18-diene and 1,3-dioxol-5-ylurazole.

Moreover, novel compounds of the invention include products and derivatives thereof obtained with subsequent reactions of 29-olefins of betulin such as with an alkylation reaction or an ene reaction, such as derivatives of betulin succinate, phenols, and polyphenols.

Preferable novel betulin derivatives according to the invention include compounds selected from the group consisting of betulin 3-acetoxime-28-nitrile, betulin 28-acetic acid methylester, 28-aspartateamide dimethylester of betulonic acid, betulin 28-N-acetylanthranilic acid ester, Diels-Alder adduct of 3β,28-diacetoxylupa-12,18-diene and urazole, Diels-Alder adduct of 3β,28-diacetoxylupa-12,18-diene and 4-methylurazole, Diels-Alder adduct of 3β,28-diacetoxylupa-12,18-diene and p-fluoro-4-phenylurazole, Diels-Alder adduct of 3β,28-diacetoxylupa-12,18-diene and m-methoxy-4-phenylurazole, Diels-Alder adduct of 3β,28-diacetoxylupa-12,18-diene and 1-naphthylurazole, and Diels-Alder adduct of 3β,28-diacetoxylupa-12,18-diene and 1,3-dioxol-5-ylurazole.

Particularly preferable novel compounds with considerable activity against leishmaniasis are the following compounds: Betulin 3-acetoxime-28-nitrile, betulin 28-acetic acid methylester, 28-aspartateamide dimethylester of betulonic acid, betulin 28-N-acetylanthranilic acid ester, Diels-Alder adduct of 3β,28-diacetoxylupa-12,18-diene and urazole, Diels-Alder adduct of 3β,28-diacetoxylupa-12,18-diene and 4-methylurazole, Diels-Alder adduct of 3β,28-diacetoxylupa-12,18-diene and 1-naphthylurazole, and Diels-Alder adduct of 3β,28-diacetoxylupa-12,18-diene and 1,3-dioxol-5-ylurazole.

Substituents present in the novel betulin derivatives defined above are often derived from naturally occurring substances or known compounds with low toxicity, or both, or said substituents are typical heterocyclic pharmacophoric moieties. Several of these compounds derived from betulin are environmentally acceptable compounds having only weak potential negative effects on the user and environment, said negative effects being also more predictable that those of synthetic compounds. Decomposition of compounds derived from betulin typically yields betulin or acid derivatives thereof, and further, constituents of substituents, Decomposition pathways of constituents, such as natural substances, present as structural moieties in the compounds and products thus generated are well known. Moreover, the toxicity of betulin derivatives is low as demonstrated by the cytotoxicity studies performed in the examples below.

Here, compounds useful according to the invention also refer to salts, and particularly pharmaceutically acceptable salts thereof. Pharmaceutically acceptable salts are obtained according to the invention by known methods using bases or acids.

Compositions to be administered to humans or animals affected by a disease caused by a protozoa of the Leishmania genus or leishmaniasis, or for the prevention of a disease caused by a protozoa of the Leishmania genus in individuals staying or travelling in areas where said disease is found or protozoa is present may be formulated from the compounds derived from betulin according to the invention.

A composition against protozoa of the Leishmania genus may be prepared from the above betulin compounds, said compositions comprising from 0.01 to 80% weight of at least one betulin derived compound, and optionally one or more substances selected from adjuvants and excipients. As adjuvants and excipients, substances known in pharmaceutical products may be used. Suitable excipients include alcohols, polyols, and polyol esters, various gels and fats, vegetable oils and solid excipients not hazardous to health such as starch, chitosan and cellulose and derivatives thereof, kaolin, talcum, and the like. Suitable vegetable oils include for example arachis, mandelic, soybean, corn, wheat germ, sesame seed, poppy seed, rapeseed, colza, tall, sunflower, palm, and olive oils.

The compositions may be formulated by methods known as such in the art e.g. into tablets, capsules, injectable liquids, suspensions, powders, and the like. The present betulin compounds may be emulsified, dissolved, or mixed in water, or in adjuvants and excipients used in the art using known mixing and production processes and additives such as surfactants, emulsifying agents, dispersants, and solvents, optionally while heating.

Particularly betulin derivatives of the invention having alkyl groups with long chains as substituents have a superior emulsifiability and/or solubility and/or miscibility in water or alcohols, polyols or polyol esters, various gels and fats, or vegetable oils or fatty acid derivatives thereof.

Daily dose of the compound derived from betulin, or a mixture thereof may suitably be from 0.005 to 5 g.

The compositions may be formulations to be administered through oral, topical, subcutaneous, intramuscular, or intravenous routes, and further, they may contain pharmaceutically acceptable adjuvants, additives, solvents and vehicles known in the art.

The betulin derivatives useful according to the invention are typically biodegradable like betulin.

The solution according to the invention has several advantages. Being nontoxic, the betulin derivatives defined above are suitable for pharmaceutical use in mammals. The compounds are biodegradable leaving no detrimental decomposition residues in nature. In addition, only targeted organisms are very specifically affected by the compounds. According to the desired application, the selectivity and decomposition rate of the agent may be controlled by substituents of betulin. If necessary, a compound decomposing more slowly, releasing the active component during decomposition, may be prepared, resulting in a uniform activity for a longer time or so-called “modified/controlled release” activity.

Betulin derivatives of the invention described above may be produced by methods I-XIV presented below.

### Method I

Betulin esters of the type IB or IFb described above may be produced by reacting 1 mol of betulin with 0.8-1.5 moles, preferably 1-1.2 moles of a C₄-C₂₂ alkyl or alkenyl derivative of maleic anhydride in the presence of imidazol (1-7 moles, preferably 3-5 moles), and a solvent at 0 to 100° C., preferably at 20 to 70° C., for 5 to 100 hours, preferably 10 to 50 h. C₁₈ alkenyl succinic anhydride (ASA) is preferably used. N-methyl-2-pyrrolidon (NMP), N,N-dimethylformamide (DMF), dimethylsulfoxide (DMSO), 1,4-dioxane, diethyl ether, tetrahydrofuran (THF), acetone, ethyl acetate, hydrocarbons and/or chlorinated hydrocarbons or mixtures thereof, preferably NMP, may serve as the solvent. After completion of the reaction, the reaction mixture is allowed to cool to room temperature, followed by separation of the product for instance by pouring the mixture into water, decanting, dissolving in a solvent, and then if necessary, washing the product with a diluted hydrochloric acid solution and water. The solvent is removed e.g. by evaporation to dryness, thus yielding desired betulin ester as the crude product that may be purified by crystallization, chromatography, or preferably by extraction using diethyl ether, tetrahydrofuran, 1,4-dioxane, 1,2-dimethoxy ethane, ethyl acetate, hydrocarbons and/or chlorinated hydrocarbons or mixtures thereof as the solvent, Esters corresponding to the structure IFb are obtained as the main product in case an excess of anhydride (1.6 to 5 moles, preferably 2 to 2.5 moles) is used, while the use of 1 to 1.2 moles of the anhydride yields esters corresponding to the structure M.

### Method II

Betulin esters having structures of types IA, IC, ID, IE, IFa, IFc, IFd and IFe described above may be produced from betulin (1 mol) and carboxylic acids (0.8 to 1.5 moles, preferably 1 to 1.2 moles) in the presence of N,N-dimethylamino pyridine (DMAP) (0.01 to 1 mol) and dicyclohexyl carbodiimide (DCC) (0.8 to 1.5 moles, preferably 1 to 1.2 moles), or N-(3-dimethylaminopropyl)-N′-ethylcarbodiimide hydrochloride (EDC) (0.8 to 1.5 moles, preferably 1 to 1.2 moles) and a solvent, by agitating at 0 to 60° C., preferably at 20 to 40° C. for 2 to 50 hours, preferably for 5 to 25 hours. The carboxylic acid is selected for different compound types as follows; IA; HO(C═O)R_{f} where R_{f}═C₁₁-C₂₂ linear or branched alkyl or alkenyl group; IC: ornithine, nicotine, N-acetylanthranilic acid or trimethyl glycine; ID: HO(C═O)CRₓ(NHR_{y}); Rₓ=alkyl, heteroalkyl, or arylalkyl group; R_{y}═H or acyl group; and IE: a carboxymethoxy derivative of verbenol, terpineol, thymol, carvacrol, menthol, cinnamic alcohol, curcumin, eugenol, borneol, isoborneol, longifolol, isolongifolol, globulol, epiglobulol, sedrol, or episedrol; or chrysanthemic acid, cinnamic acid, or retinolic acid. NWT, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably dichloromethane, may serve as the solvent. After completion of the reaction, the reaction mixture is poured into water, organic layer is separated, followed by removing the solvent for instance by evaporation to dryness, thus yielding betulin ester as the crude product that may be purified if necessary by crystallization, chromatography, or extraction, preferably by extraction. Use of 0.8 to 1.5 moles of the carboxylic acid reagent results in compounds having the structures IA, IC, ID, IE or IFd while use of an excess of the carboxylic acid reagent (1.6 to 3 moles, preferably 2 to 2.5 moles) with dicyclohexyl carbodiimide (DCC) (1.6 to 3 moles, preferably 2 to 2.5 moles), or with N-(3-dimethylaminopropyl)-N′-ethylcarbodiimide hydrochloride (EDC) (1.6 to 3 moles, preferably 2 to 2.5 moles) yields compounds corresponding to structures IFa, IFe, IFd, or IFe. For the production of the compounds of the IE or IFe type, an acetic acid derivative of the alcohol used as starting material is first generated according to method V.

### Method III

Betulin esters having structures of types IA, IC, IE, IFa, IFe and IFd described above may be produced from betulin (1 mol) with carboxylic acids (0.8 to 1.5 moles, preferably 1 to 1.2 moles) in the presence of a tetraisopropyl ortho titanate, tetrabutyl ortho titanate, p-toluenesulfonic acid monohydrate, or pyridine-p-toluenesulfonate catalyst (0.01 to 1 mol), or sulphuric acid or hydrochloric acid (1 to 6%, preferably 2 to 4%) and a solvent, by agitating at 80 to 160° C., preferably at 100 to 140° C. for 2 to 50 hours, preferably for 4 to 25 hours. The carboxylic acid is selected for different compound types as follows: IA: HO(C═O)Rᵢ where Rᵢ═C₁₁-C₂₂ linear or branched alkyl or alkenyl group; IC: ornithine, nicotine, N-acetylanthranilic acid or trimethyl glycine; IE: a carboxymethoxy derivative of verbenol, terpineol, thymol, carvacrol, menthol, cinnamic alcohol, curcumin, eugenol, borneol, isoborneol, longifolol, isolongifolol, globulol, epiglobulol, sedrol, or episedrol; or chrysanthemic acid, cinnamic acid, or retinolic acid, Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably toluene or xylene, may serve as the solvent. Water generated in the reaction is separated using a water separator tube, or vacuum. After completion of the reaction, the reaction mixture is poured into water, organic layer is separated, washed if necessary with a basic aqueous solution, preferably with an aqueous NaHCO₃ or Na₂CO₃ solution, followed by removing the solvent for instance by evaporation to dryness, thus yielding betulin ester as the crude product that may be purified if necessary by crystallization, chromatography, or extraction, preferably by extraction. Use of 0.8 to 1.5 moles of the carboxylic acid reagent results in compounds having the structures IA, IC, or IE while use of an excess of the carboxylic acid reagent (1.6 to 3 moles, preferably 2 to 2.5 moles) yields compounds corresponding to structures IFa, IFe, or IFe. For the production of the compounds of the IE or IFe type, an acetic acid derivative of the alcohol used as starting material is first generated according to method V.

### Method IV

Esters having structures of types IA, IC, ID, IE, IFa, IFc, IFd, and IFe described above may be produced from betulin (1 mol) and carboxylic acids (0.8 to 1.5 moles, preferably 1 to 1.2 moles), first allowed to react with oxalyl chloride or thionyl chloride (1 to 10 moles, preferably 1 to 4 moles) without or in the presence of a solvent, by agitating at 0 to 80° C., preferably at 20 to 50° C. for 2 to 50 hours, preferably for 5 to 25 hours. The carboxylic acid is selected for different compound types as follows: IA: HO(C═O)Rᵢ where Rᵢ═C₁₁-C₂₂ linear or branched alkyl or alkenyl group; IC; ornithine, nicotine, N-acetylanthranilic acid or trimethyl glycine; ID: HO(C═O)CRₓ(NHR_{y}); Rₓ=alkyl, heteroalkyl, or arylalkyl group; R_{y}═H or acyl group; and IE: a carboxymethoxy derivatives of verbenol, terpineol, thymol, carvacrol, menthol, cinnamic alcohol, curcumin, eugenol, borneol, isoborneol, longifolol, isolongifolol, globulol, epiglobulol, sedrol, or episedrol; or chrysanthemic acid, cinnamic acid, or retinolic acid. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably dichloromethane, may serve as the solvent. After completion of the reaction, the solvent is removed for instance by evaporation to dryness, if necessary, followed by purification of the desired acid chloride by crystallization, chromatography, or extraction, preferably by extraction. The acid chloride (0.8 to 1.5 moles, preferably 1 to 1.2 moles) thus obtained is reacted with betulin (1 mol), base (0.5 to 10 moles, preferably 1 to 5 moles) such as triethyl amine, tripropyl amine, diisopropylethyl amine, preferably triethyl amine in the presence of a solvent, or in the presence of the DMAP catalyst (0.001 to 1 mol), pyridine and solvent, or with a base (0.5 to 10 moles, preferably 1 to 5 moles) such as triethyl amine, tripropyl amine, diisopropylethyl amine, preferably triethyl amine, and pyridine by agitating at 0 to 80° C., preferably at 20 to 50° C. for 2 to 50 hours, preferably for 5 to 25 hours. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably dichloromethane, may serve as the solvent. After completion of the reaction, betulin amide or betulin ester product is purified by crystallization, chromatography, or extraction, preferably by extraction, if necessary. Use of 0.8 to 1.5 moles of the acid chloride reagent results in compounds having the structures IA, IC, ID, or IE while use of an excess of the acid chloride reagent (1.6 to 3 moles, preferably 2 to 2.5 moles) yields compounds corresponding to structures IFa, IFe, IFd, or IFe. For the production of the compounds of the IE or IFe type, an acetic acid derivative of the alcohol used as starting material is first generated according to method V,

### Method V

For the production of betulin derivatives having structures of the IE and IFe type according to the methods II, III or IV, and betulin derivatives having structures of the IIa and IIb type according to the method IV, an acetic acid derivative of the alcohol is first generated as follows. Acetic acid derivative is produced by mixing an alcohol (1 mol) and chloroacetic acid (0.8 to 1.5 moles, preferably 1 to 1.2 moles) in water for 1 to 7 hours, preferably for 3 to 5 hours, at 100 to 150° C. preferably at 120-130° C., in the presence of lithium, potassium, sodium, or hydrides or hydroxides thereof (1.5 to 3 moles, preferably 1.8 to 2.2 moles), preferably sodium (Na), sodium hydride (NaH), or sodium hydroxide (NaOH). The alcohol is selected from the group consisting of verbenol, terpineol, thymol, carvacrol, menthol, cinnamic alcohol, curcumin, eugenol, borneol, isoborneol, longifolol, isolongifolol, globulol, epiglobulol, sedrol, and episedrol. The mixture is allowed to cool to room temperature, made acidic with concentrated hydrochloric acid, and extractor with a solvent. Hydrocarbons and/or chlorinated hydrocarbons, diethyl ether, tetrahydrofuran, 1,4-dioxane, 1,2-dimethoxy ethane, ethyl acetate, or mixtures thereof, preferably diethyl ether, may serve as the solvent. If necessary, the organic phase is washed with a basic aqueous solution, preferably with an aqueous NaHCO₃ or Na₂CO₃ solution. The solvent is removed for instance by evaporation to dryness, thus yielding a carboxymethoxy intermediate that may be purified if necessary by crystallization, chromatography, or extraction, preferably by extraction.

### Method VI

Derivatives of types IG, IH, II, and IJ described above may be produced from betulonic acid (1 mol) and natural alcohols (0.8 to 1.5 moles, preferably 1 to 1.2 moles), or amino acids (0.8 to 1.5 moles, preferably 1 to 1.2 moles), in the presence of a solvent and DMAP (0.001 to 1 moles) and DCC (0.8 to 1.5 moles, preferably 1 to 1.2 moles), or EDC (0.8 to 1.5 moles, preferably 1 to 1.2 moles), by agitating at 0 to 60° C., preferably at 20-50° C. for 2 to 50 hours, preferably for 5 to 25 hours. For the different compound types, the alcohol is selected as follows: IH; verbenol, terpineol, thymol, carvacrol, menthol, cinnamic alcohol, curcumin, eugenol, borneol, isoborneol, longifolol, isolongifolol, globulol, epiglobulol, sedrol, and episedrol. For the different compound types, the amino acid is selected as follows: IG; HO(C═O)Rₜ where Rₜ═NHCHRₓCOOY where Y═H, Na, K, Ca, Mg, C₁-C₄-alkyl group or NRₓ where Rₓ═H, benzyl, 4-hydroxybenzyl, —CH₂CH₂CH₂CH₂NH₂, 4-imidazolyl methyl, 3-indolyl methyl or CH₃SCH₂ group; preferably dimethyl ester hydrochloride of aspartic acid, methyl ester hydrochloride of L-histidine, dimethyl ester hydrochloride of L-glutaminic acid or methyl ester dihydrochloride of L-lysine. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably dichloromethane, may serve as the solvent. After completion of the reaction, the to desired betulonic acid amide or ester product (of the type IJa or IJb) may be purified by crystallization, chromatography, or extraction, preferably by extraction, if necessary. The betulonic acid amide or ester thus obtained may be reduced to the corresponding betulinic acid amide or ester product (of the type IG or IH) if desired using sodium borohydride according to U.S. Pat. No. 6,280,778. After completion of the reaction, said betulinic acid amide or ester may be purified by crystallization, chromatography, or extraction, preferably by extraction, if necessary. Betulin derivatives of the IIa and III) type are obtained by reacting the betulinic acid amide or ester thus obtained as described in the methods II, III or IV.

### Method VII

Compounds having structures of the types IG, III, II, and IJ described above may be produced from betulonic acid (1 mol) by reacting with oxalyl chloride or thionyl chloride (1 to 10 moles, preferably 1 to 4 moles) without, or in the presence of a solvent by agitation at 0 to 80° C., preferably 20 to 50° C., for 2 to 50 hours, preferably for 5 to 25 hours. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably dichloromethane, may serve as the solvent. After completion of the reaction, the desired acid chloride may be purified by crystallization, chromatography, or extraction, preferably by extraction, if necessary. Betulonic acid chloride thus obtained from the reaction (1 mol) is reacted with an amino acid (0.8 to 1.5 moles, preferably 1 to 1.2 moles), or an alcohol (0.8 to 1.5 moles, preferably 1 to 1.2 moles), with a base such as triethyl amine, tripropyl amide diisopropyl ethyl amine, pyridine, preferably triethyl amine in the presence of a solvent, or in the presence of the DMAP catalyst (0.001 to 1 mol), pyridine and solvent, or with a base (0.5 to 10 moles, preferably 1 to 5 moles) such as triethyl amine, tripropyl amine, diisopropylethyl amine, preferably triethyl amine, and pyridine by agitating at 0 to 80° C., preferably at 20 to 50° C. for 2 to 50 hours, preferably for 5 to 25 hours. For the different compound types, the amino acid is selected as follows: IG: HO(C═O)Rₜ where Rₜ═NHCHRₓCOOY where Y═H, Na, K, Ca, Mg, C₁-C₄-alkyl group or NRₓ where Rₓ═H, C₁-C₄-alkyl, benzyl, 4-hydroxybenzyl, —CH₂CH₂CH₂CH₂NH₂, 4-imidazolyl methyl, 3-indolyl methyl, or CH₃SCH₂ group; preferably dimethyl ester hydrochloride of aspartic acid, methyl ester hydrochloride of L-histidine, dimethyl ester hydrochloride of L-glutaminic acid, and methyl ester dihydrochloride of L-lysine. For the different compound types, the alcohol is selected as follows; IH: verbenol, terpineol, thymol, carvacrol, menthol, cinnamic alcohol, curcumin, eugenol, borneol, isoborneol, longifolol, isolongifolol, globulol, epiglobulol, sedrol, and episedrol, Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably dichloromethane, may serve as the solvent. After completion of the reaction, the reaction mixture is washed with diluten hydrochloric acid solution and water. The solvent is evaporated to dryness, and the reaction product (of the type IJa or IJb) is purified by crystallization, chromatography, or extraction, preferably by extraction, if necessary. The betulonic acid amide or ester product thus obtained may be reduced to the corresponding betulinic acid amide or ester product (of the type IG or IH) using sodium borohydride according to U.S. Pat. No. 6,280,778. After completion of the reaction, the desired betulinic acid amide or ester is purified by crystallization, chromatography, or extraction, preferably by extraction, if necessary. Betulin derivatives of the II type are obtained by reacting the betulinic acid amide or ester thus obtained as described in the methods II, III or IV.

### Method VIII

Compounds having structures of the type IK described above may be produced from betulin (1 mol) and aromatic compounds selected to have R_{z}═C₆H₅₋ₙ(OH)ₙ or C₆H₅₋ₙ₋ₘ(OH)ₙ(OCH₃)ₘ and n=0-5, m=0-5, n+m≦5 (4 to 20 moles) as the phenol residue in the IK group, in the presence of a polymeric acid catalyst, preferably a sulfonic acid derivative of polystyrene (0.1 to 1.5 g, preferably 0.5 to 1 g, 16 to 50 mesh) and a solvent. The reaction mixture is agitated in an inert atmosphere at 20 to 120° C., preferably at 75 to 110° C. for 1 to 5 hours, preferably for 2 to 4 hours. Water generated in the reaction is suitably separated using water separating tube or vacuum. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably hydrocarbons and/or chlorinated hydrocarbons or ether may serve as the solvent. After completion of the reaction, the mixture is allowed to cool to room temperature, filtered, the filtrate is washed with water, dried, and the solvent is separated. The betulin derivative thus obtained is purified by crystallization, chromatography, or extraction, preferably by extraction, if necessary.

### Method IX

Compounds having structures of the type IL described above may be produced from compounds having structures of the type IA or IFa prepared as described in the methods II, III, or IV, and maleic anhydride (0.8 to 10 moles, preferably 1 to 5 moles), in the presence of hydrochinone (0.05 to 0.5 moles, preferably 0.08 to 0.3 moles), and a solvent, or in a melt by heating the reaction mixture at 150 to 220° C., preferably at 160 to 180° C. for 1 to 5 hours, preferably for 2 to 4 hours. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof may serve as the solvent, preferably as a melt. After Completion of the reaction, the desired product is purified by crystallization, chromatography, or extraction, preferably by extraction, if necessary. The anhydride derivative of betulin thus obtained may be further converted into an imide or ester compound having the structure of the type IL using known methods.

### Method X

Betulin derivatives having structures of the types IM, IN, IO, IP and IQ described above may be produced by reacting betulin (1 mol) in the presence of triphenylphosphine (0.8 to 8 moles, preferably 2 to 5 moles), 3,3-dimethylglutaric imide (0.8 to 8 moles, preferably 2 to 5 moles), diethylazo dicarboxylate solution (0.8 to 8 moles, preferably 2 to 5 moles), and a solvent by agitating at 0 to 60° C., preferably at 20 to 40° C. for 2 to 5 hours, preferably for 5 to 25 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably tetrahydrofuran, may serve as the solvent. After completion of the reaction, the precipitate formed is filtered off. The solvent is removed for instance by evaporation to dryness, thus yielding 3-deoxy-2,3-dihydro betulin as the crude product that may be purified by crystallization, chromatography, or extraction, preferably by extraction, if necessary.

### Method XI

Betulin derivatives having structures of the types IN and IO described above may be produced by reacting betulin (1 mol) with a Diels-Alder adduct (0.8 to 5 moles, preferably 1 to 2 moles), diphenylphosphoryl azide (DPPA) (0.8 to 5 moles, preferably 1 to 2 moles), and with a base, triethyl amine, tripropyl amine, diisopropylethyl amine, preferably triethyl amine (TEA) (0.8 to 5 moles, preferably 1 to 2 moles), in the presence of a solvent, by agitating at 0 to 150° C., preferably 60 to 120° C. for 1 to 48 hours, preferably for 2 to 24 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons or mixtures thereof, preferably toluene, may serve as the solvent. After completion of the reaction, the reaction mixture is washed with diluted aqueous basic solution, diluted acidic solution, water, if necessary, followed by removal of the solvent for instance by evaporating to dryness. 28-O-Diels-Alder adduct of betulin is obtained as the crude product that may be purified by crystallization, chromatography, or extraction, preferably by crystallization, if necessary. Use of an excess of the Diels-Alder adduct, diphenylphosphoryl azide (DPPA) and triethyl amine (1.5 to 3 moles, preferably 2 to 2.2 moles) results in 3,28-O-Diels-Alder diadduct of betulin.

Diels-Alder adducts may be produced from a C₅-C₂₂ diene acid (1 mol) that may be linear, branched, cyclic or heterocyclic comprising O, N or S as a hetero atom, preferably by reacting 2,4-pentadiene acid, sorbic acid, 2-furanoic acid or anthracene-9-carboxylic acid with a dienophile, preferably with 4-substituted triazolinedion, maleic anhydride, N-substituted maleimide, diethylazodicarboxylate or dimethylacetylene dicarboxylate (0.5 to 5 moles, preferably 0.8 to 2 moles) in the presence of a solvent while agitating at 0 to 150° C., preferably at 20 to 120° C. for 1 to 48 hours, preferably for 2 to 24 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons or mixtures thereof, preferably toluene, may serve as the solvent. The reaction may also be performed without any added solvent. After completion of the reaction, the reaction mixture is washed with water, if necessary, followed by removal of the solvent by e.g. evaporation to dryness, A Diels-Alder adduct is obtained as the crude product that may be purified by crystallization, chromatography, or extraction, preferably by crystallization, if necessary.

### Method XII

Betulin derivatives having structures of the types IN and IO described above may be produced by protecting the C28 hydroxyl group of betulin (1 mol) with a substituted methyl ether, substituted ethyl ether, substituted phenyl ether, silyl ether, ester, carbonate, or sulfonate using known methods, preferably with dihydropyran (DHP) (0.8 to 8 moles, preferably 1 to 2 moles), in the presence of pyridinium-p-toluene sulfonate (PPTS) (0.01 to 2 moles, preferably 0.05 to 5 moles) and a solvent while mixing at 0 to 60° C., preferably at 20 to 40° C. for 5 to 100 hours, preferably for 12 to 48 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably dichloromethane, may serve as the solvent. After completion of the reaction, the organic phase is washed with saturated aqueous solution of a base, and with water. The solvent is e.g. removed by evaporation to dryness yielding a betulin derivative as crude product having the C28 hydroxyl group protected with substituted methyl ether, substituted ethyl ether, substituted phenyl ether, silyl ether, ester, carbonate, or sulfonate, preferably with dihydropyran. The crude product, preferably betulin 28-tetrahydropyran ether may be purified by crystallization, chromatography, or extraction, preferably by extraction, if necessary.

Betulin derivative having the C28 hydroxyl group protected with substituted methyl ether, substituted ethyl ether, substituted phenyl ether, silyl ether, ester, carbonate, or sultanate, preferably with dihydropyran (betulin 28-tetrahydropyran ether) (1 mol) and a Diels-Alder adduct (0.8 to 5 moles, preferably 1 to 2 moles) produced according to the method XI, diphenylphosphoryl azide (DPPA) (0.8 to 5 moles, preferably 1 to 2 moles), and a base, triethyl amine, tripropyl amine, diisopropyl ethyl amine, preferably triethyl amide (TEA) (0.8 to 5 moles, preferably 1 to 2 moles) are reacted in the presence of a solvent while mixing at 0 to 150° C., preferably at 60 to 120° C. for 1 to 48 hours, preferably 2 to 24 hours, NMP, DMF, DMSO, 1,4-dioxane, diethyl ether, tetrahydrofuran, 1,2-dimethoxy ethane, acetone, ethyl acetate, Hydrocarbons and/or chlorinated hydrocarbons, CH mixtures thereof, preferably toluene, may serve as the solvent. After completion of the reaction, the reaction mixture is washed with a diluted basic solution, diluted acid solution, water, if necessary, followed by removal of the solvent e.g. by evaporation to dryness. As crude product, betulin derivative having the C28 hydroxyl group protected with substituted methyl ether, substituted ethyl ether, substituted phenyl ether, silyl ether, ester, carbonate, or sulfonate, preferably with dihydropyran, and having at C3 hydroxyl group a Diels-Alder adduct, preferably a Diels-Alder adduct of 2,4-pentadiene acid with 4-phenyl-1,2,4-triazolin-3,5-dion, is obtained. The crude product, preferably 3-O-Diels-Alder adduct of betulin 28-tetrahydropyran ether may be purified by crystallization, chromatography, or extraction, preferably by crystallization, if necessary.

C28 hydroxyl group of the betulin derivative having the C28 hydroxyl group protected with substituted methyl ether, substituted ethyl ether, substituted phenyl ether, silyl ether, ester, carbonate or sulfonate is deprotected using known methods, preferably the protecting group, tetrahydropyran, of the C28 hydroxyl of the 3-O-Diels-Alder adduct of 28-tetrahydropyran ether (1 mol) is cleaved using pyridinium-p-toluene sulfonate (PPTS) (0.02 to 1 mol, preferably 0.05 to 0.5 mol) by allowing said PPTS to react while agitating at 0 to 80° C., preferably at 20 to 40° C. for 24 to 240 hours, preferably 48 to 120 hours. NMP, DMF, DMSO, 1,4-dioxane, methanol, ethanol, 1-propanol, 2-propanol, diethyl ether, tetrahydrofuran, 1,2-dimethoxy ethane, acetone, ethyl acetate, hydrocarbons and/or chlorinated hydrocarbons or mixtures thereof, preferably methanol or ethanol may serve as the solvent. After completion of the reaction, the reaction mixture is diluted with an organic solvent, washed with a diluted aqueous solution of a base, diluted acidic solution, water if necessary, followed by removal of the solvent for instance by evaporation to dryness. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably ethyl acetate, may serve as the solvent. Betulin 3-O-Diels-Alder adduct is obtained as the product that may be purified by crystallization, chromatography, or extraction, preferably by crystallization.

### Method XIII

Heterocyclic betulin derivatives of the types IP and IQ described above may be produced by reacting betulin (1 mol) in the presence of an anhydride (1.6 to 5 moles, preferably 2 to 2.5 moles), N,N-dimethylamino pyridine (DMAP) (0.01 to 1 mol), a base, pyridine, triethyl amine, tripropyl amide, diisopropylethyl amine, preferably pyridine (1 to 100 moles, preferably 20 to 50 moles), and a solvent at 0 to 100° C., preferably at 20 to 50° C. for 5 to 100 hours, preferably 10 to 50 hours. The anhydride is preferably acetic anhydride, however, also other carboxylic anhydrides such as propionic anhydride, phthalic anhydride, or benzoic anhydride may be used. N-methyl-2-pyrrolidon (NMP), N,N-dimethylformamide (DMF), dimethylsulfoxide (DMSO), 1,4-dioxane, diethyl ether, tetrahydrofuran (THF), acetone, ethyl acetate, hydrocarbons and/or chlorinated hydrocarbons or mixtures thereof, preferably dichloromethane, may serve as the solvent. After completion of the reaction, the reaction mixture is washed, if necessary, with diluted hydrochloric acid solution, aqueous basic solution, and with water. Solvent is for instance removed by evaporation to dryness, giving 3,28-diester of betulin, preferably 3,28-diacetate of betulin as the crude product that may be purified by crystallization, chromatography, or extraction, preferably by extraction, if necessary.

The 3,28-diester of betulin (1 mol), preferably the 3,28-diacetate of betulin, may be isomerized to give 3β,28-diacetoxylupa-18-enen in the presence of hydrochloric or hydrobromic, preferably hydrobromic acid (5 to 25 Vu, preferably 10 to 15%), acetic acid (25 to 60%, preferably 35 to 50%), acetic anhydride (5 to 30%, preferably 10 to 20%), and a solvent at 0 to 60° C., preferably at 20 to 40° C. for 4 to 1200 hours, preferably for 10 to 24 hours, NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably toluene, may serve as the solvent. After completion of the reaction, the reaction mixture is washed, if necessary, with a basic aqueous solution and water, followed by removal of the solvent for instance by evaporation to dryness, 3β,28-diacetoxylup-18-ene is obtained as crude product that may be purified by crystallization, chromatography, or extraction, preferably by crystallization, if necessary,

3β,28-diacetoxylup-18-ene (1 mol) may be epoxylated using hydrogen peroxide or a peracid, preferably m-chloroperbenzoic acid (mCPBA) (0.8 to 3 moles, preferably 1 to 1.5 moles) in the presence of sodium carbonate, sodium hydrogen carbonate, sodium hydrogen phosphate, potassium carbonate, potassium hydrogen carbonate, potassium hydrogen phosphate, preferably sodium carbonate (1 to 15 moles, preferably 3 to 8 moles) and a solvent while agitating at 0 to 60° C., preferably at 20 to 40° C. for 0.5 to 10 hours, preferably 1 to 4 hours, NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably chloroform, may servo as the solvent. After completion of the reaction, the reaction mixture is washed, if necessary, with a basic aqueous solution and water, followed by removal of the solvent for instance by evaporation to dryness. 3β,28-diacetoxylup-18,19-epoxylupane is obtained as crude product that may be purified by crystallization, chromatography, or extraction, preferably by crystallization, if necessary.

3β,28-diacetoxylup-18,19-epoxylupane (1 mol) reacts to give 3β,28-diacetoxylupa-12,18-diene and 3β,28-diacetoxylupa-18,21-diene in the presence of p-toluenesulfonic acid (0.1 to 3 moles, preferably 0.3 to 1 moles) and acetic anhydride (0.5 to 5 moles, preferably 1 to 3 moles) and a solvent while agitating at 50 to 150° C., preferably at 90 to 130° C., for 0.5 to 12 hours, preferably for 2 to 5 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably toluene, may serve as the solvent. After completion of the reaction, the reaction mixture is washed, if necessary, with a basic aqueous solution and water, followed by removal of the solvent for instance by evaporation to dryness, 3β,28-diacetoxylupa-12,18-diene and 3β,28-diacetoxylupa-18,21-diene are obtained as crude products that may be purified by crystallization, chromatography or extraction, preferably by crystallization, if necessary.

A heterocyclic Diels-Alder adduct may be produced from a mixture (1 mol) of 3β,28-diacetoxylupa-12,18-diene and 3β,28-diacetoxylupa-18,21-diene by reacting said mixture with a dienophile, preferably with 4-substituted triazolindion, maleic anhydrode, N-substituted maleimide, diethylazodicarboxylate, or dimethylacetylene dicarboxylate (0.5 to 5 moles, preferably 0.8 to 2 moles) in the presence of a solvent while agitating at 0 to 150° C., preferably at 20 to 120° C., for 1 to 48 hours, preferably for 2 to 24 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably toluene, may serve as the solvent. After completion of the reaction, the reaction mixture is washed, if necessary, with water, followed by removal of the solvent for instance by evaporation to dryness. Heterocyclic Diels-Alder adduct of betulin is obtained as crude product that may be purified by crystallization, chromatography, or extraction, preferably by crystallization, if necessary,

### Method XIV

Substances having structures of the types IP described above may be produced by adding isocyanate (0.5 to 5 moles, preferably 0.8 to 1.5 moles) to ethylhydrazine (1 mol) in the presence of a solvent. The isocyanate R—N═C═O is selected from the group where R═H, C₁-C₆ linear or branched alkyl or alkenyl group or aromatic group ZZ of the formula

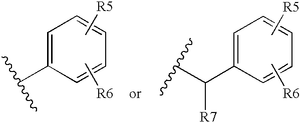

where R5, R6 and/or R7 may represent H, C₁-C₆ linear or branched alkyl or alkenyl group or C₁-C₆ linear or branched alkyl or alkenyl ether, R5-R6 forms a cyclic C₂-C₆-alkyl or alkenyl group, halogen (fluoro, chloro, bromo, iodo), nitro, carboxy, carboxyl, acetyl, R5-R6 forms a cyclic methylenedioxy group, sulfate, cyano, hydroxy, or trifluoromethyl, NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably toluene, may serve as the solvent. The reaction mixture is agitated at 0 to 60° C., preferably at 0 to 40° C., for 0.5 to 12 hours, preferably for 1 to 5 hours, and 40 to 120° C., preferably at 60 to 100° C., for 0.5 to 12 hours, preferably for 1 to 5 hours. After completion of the reaction, the crude product formed is filtered and dried. The crude product, 4-substituted 1-carbethoxy semicarbazide may be purified by crystallization, chromatography, or extraction, preferably by extraction, if necessary.

Said 4-substituted 1-carbethoxy semicarbazide (1 mol) may be cyclized to give 4-substituted urazole by heating in an aqueous NaOH or KOH solution, preferably in aqueous KOH solution (1 to 10 M, preferably 2 to 6 M) at 40 to 100° C., preferably 50 to 80° C., for 0.5 to 6 hours, preferably Ito 3 hours. The reaction mixture is filtered, followed by precipitation of the crude product with concentrated HCl solution, filtered and dried for instance in an oven or desiccator. The crude material, 4-substituted urazole, may be purified by crystallization, chromatography, or extraction, preferably by crystallization, if necessary.

Said 4-substituted urazole (1 mol) is oxidized using iodobenzene diacetate (0.5 to 6 moles, preferably 0.8 to 1.5 moles) in the presence of a solvent while agitating at 0 to 80° C., preferably at 20 to 40° C. for 0.1 to 4 hours, preferably 0.2 to 1 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably tetrahydrofuran or dichloromethane, may serve as the solvent. A mixture of 3β,28-diacetoxylupa-12,18-diene and 3β,28-diacetoxylupa-18,21-diene produced according to the method XIII (0.2 to 2 moles, preferably 0.8 to 1.2 moles) is added to the reaction mixture), followed by agitating said reaction mixture at 0 to 60° C., preferably at 0 to 40° C., for 1 to 48 hoursm preferably for 2-to 24 hours, and then, the solvent is removed e-g- by evaporation to dryness. The crude product, a Diels-Alder adduct of the 4-substituted urazole, may be purified by crystallization, chromatography, or extraction, preferably by crystallization.

The invention is now illustrated by the following examples without wishing to limit the scope thereof.

### EXAMPLES

### Example 1

### Preparation of the 28-C₁₈ Alkylene Succinic Ester of Betulin

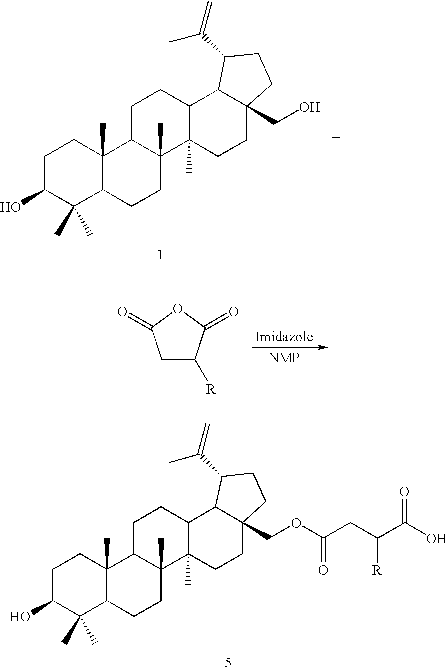

Imidazole (38.8 mmol) and C₁₈ alkylene succinic anhydride (ASA) 4 (11.6 mmol) were agitated in NMP (25 ml). Betulin 1 (9.7 mmol) was added, followed by further agitation at room temperature for 3 days. The organic phase was poured into water, decanted, dissolved in dichloromethane, and washed. The solvent was evaporated, thus yielding 28-C₁₈ alkylene succinic ester of betulin 5 (yield; 73%).

### Example 2

### Preparation of the 3,28-C₁₈ Alkylene Succinic Diester of Betulin

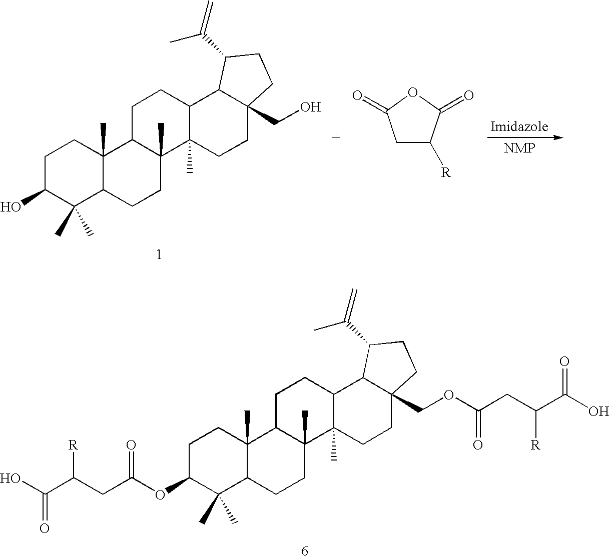

Imidazole (54.2 mmol) and C₁₈ alkylene succinic anhydride (ASA) 4 (32.5 mmol) were agitated in NMP (30 ml). Betulin 1 (13.5 mmol) was added, followed by further agitation at room temperature for 3 days. The organic phase was poured into water, decanted, dissolved in dichloromethane, and washed. The solvent was evaporated, thus yielding 3,28-C₁₈ alkylene succinic diester of betulin 6 (yield; 40%).

### Example 3

### Preparation of the 28-carboxymethoxy Mentholester of Betulin

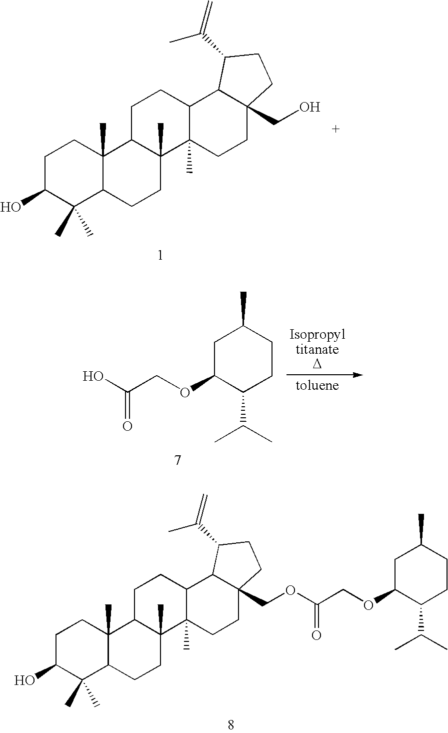

Betulin 1 (11.7 mmol) and menthoxyacetic acid 7 (11.7 mmol) were weighed in a flask, followed by the addition of toluene (120 ml) as the solvent. The mixture was heated to 120° C., and added with isopropyl titanate (1.4 mmol). The reaction mixture was refluxed for 3 h until water was separated by the water separation tube. The mixture was cooled to room temperature and the precipitate formed was filtered. The organic phase was washed and the solvent was evaporated, yielding 28-carboxymethoxy mentholester of betulin 8 (yield: 60%).

### Example 4

### Preparation of the 28-carboxymethoxy Carvacrolester of Betulin

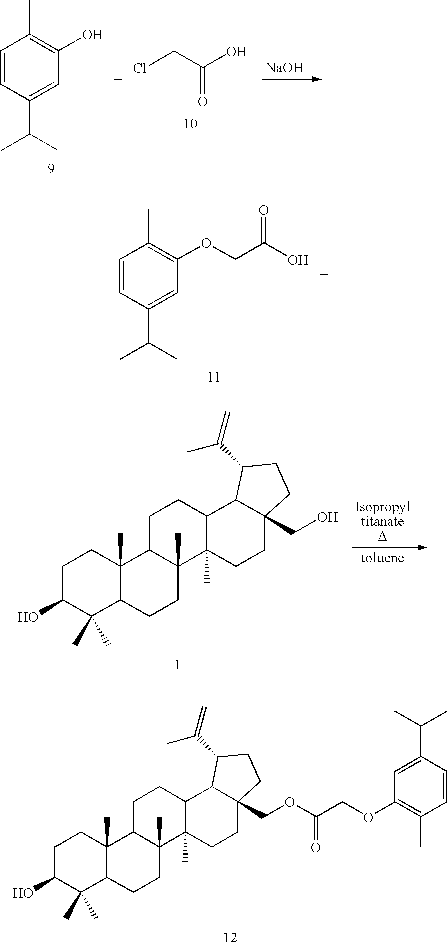

NaOH beads (66.6 mmol) were added to a mixture of carvacrol 9 (33.3 mmol), chloroacetic acid 10 (33.3 mmol) and water (50 ml). The mixture was refluxed at 120° C. for 3 h. The mixture was cooled to room temperature and acidified with hydrochloric acid. The crude product was extracted with diethyl ether and washed with water. The solvent was evaporated, thus giving carvacrol oxyacetic acid 11 (yield: 83%). The crude product was purified by dissolving in diethyl ether, followed by extraction with water and NaHCO₃ solution, Aqueous phases were pooled, acidified with hydrochloric acid and extracted with diethyl ether. The ether phase was dried, followed by evaporation of the solvent to dryness, thus giving carvacrol acetic acid 11 (yield: 45%), Betulin 1 (7.2 mmol) and carvacrol oxyacetic acid 11 (7.2 mmol) were weighed into a flask, and toluene (80 ml) was added. The bath was heated to 160° C., and then isopropyl titanate (1.4 mmol) was added. The reaction mixture was refluxed for 6 h until all water was separated by the water separation tube. The mixture was cooled to room temperature and the precipitate formed was filtered. The organic phase was washed with NaHCO₃ solution and the solvent was evaporated. The crude product was recrystallized from boiling solution of cyclohexane and toluene. The solvent was evaporated to dryness, thus isolating 28-carboxymethoxy carvacrolester of betulin 12 (yield: 55%) as the reaction product.

### Example 5

### Preparation of the 28-cinnamon Alcohol Acetic Acid Ester of Betulin

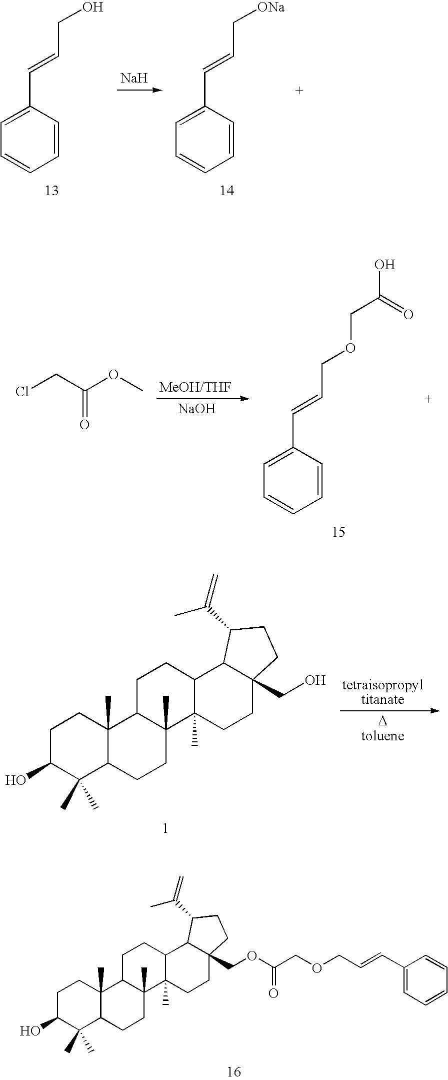

A mixture of sodium hydride (8.2 mmol) and tetrahydrofuran was added with cinnamon alcohon 13 (7.5 mmol), and agitation was continued for 1 h. Methylchloroacetate (7.5 mmol) was added to the reaction flask, and agitation was continued for 24 hours. The reaction mixture was diluted with diethyl ether, and then the organic phase was washed with water and dried. The solvent was evaporated to dryness, and the precipitate was dissolved in a solution of methanol and tetrahydrofuran. Sodium hydroxide solution (10.9 mmol) was added, and the reaction mixture was refluxed for 4 hours. The solvent was evaporated. Water was added to the flask, acidified with hydrochloric acid, and extracted with diethyl ether. The organic phase was washed with water, and the solvent was evaporated, thus giving cinnamic acid 15 (yield: 23%). Betulin 1 (0.9 mmol) and cinnamic acid 15 (0.9 mmol) were weighed into a flask, and toluene (40 ml) was added as the solvent. The bath was heated to 160° C., and then isopropyl titanate (0.2 mmol) was added to the reaction mixture. The reaction mixture was refluxed for 4.5 h until all water was separated by the water separation tube. The mixture was cooled to room temperature and the precipitate formed was filtered. The organic phase was washed with NaHCO₃ solution and the solvent was evaporated. The crude product was recrystallized from boiling solution of cyclohexane and toluene. After the mixture was cooled, the crystallized precipitate was filtered. The solvent was evaporated to dryness, thus giving 28-cinnamon alcohol acetic acid ester of betulin 16 (yield: 14%) as the reaction product.

### Example 6

### Preparation of 28-eugenolester of Betulonic Acid

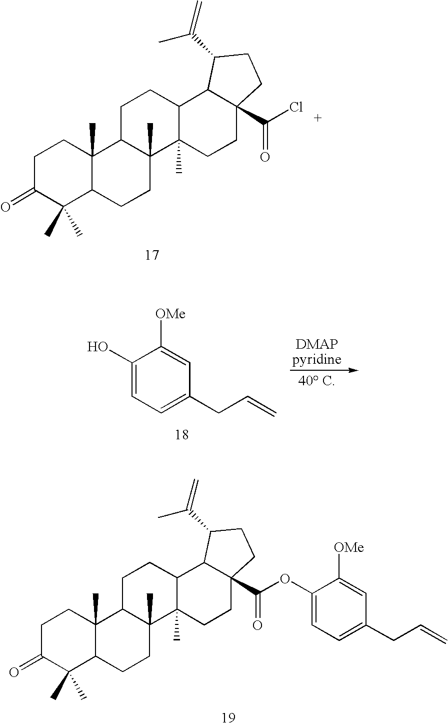

A mixture of betulonic acid chloride 17 (1.4 mmol) (prepared as described in example 12), eugenol 18 (1.1 mmol), DMAP (1.1 mmol), and pyridine was heated for 48 hours at 40° C. The reaction mixture was diluted with toluene, washed with diluted hydrochloric acid solution, and water and then dried over sodium sulfate. The solvent was evaporated, thus giving 28-eugenol ester of betulonic acid 19 (yield: 81%).

### Example 7

### Preparation of 28-carboxymethoxythymol Ester of Betulin

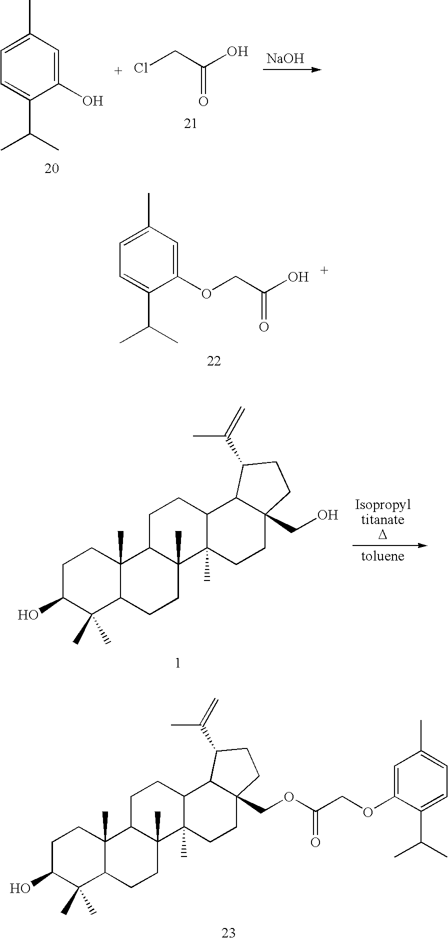

NaOH beads (66.6 mmol), dissolved in water, were added to a mixture of thymol 20 (33.3 mmol), chloroacetic acid 21 (33.3 mmol) and water. The mixture was refluxed at 120° C. for 3 h. The mixture was cooled to room temperature, acidified, extracted with diethyl ether and washed. The solvent was evaporated thus giving precipitated thymolacetic acid 22 with a yield of 29%. Betulin 1 (7.2 mmol), thymolacetic acid 22 (7.2 mmol), and toluene (80 ml) were heated to 160° C., followed by the addition of isopropyl titanate (1.4 mmol) to the reaction mixture. The reaction mixture was refluxed for 4.5 h until all water was separated by the water separation tube. The mixture was cooled to room temperature and the precipitate formed was filtered. The organic phase was washed and the solvent was evaporated. The crude product was recrystallized from boiling solution of cyclohexane and toluene (3.5:1), thus giving 28-carboxymethoxythymol ester of betulin 23 (yield: 61%) as the reaction product.

### Example 8

### Preparation of 28-chrysanthemate of Betulin

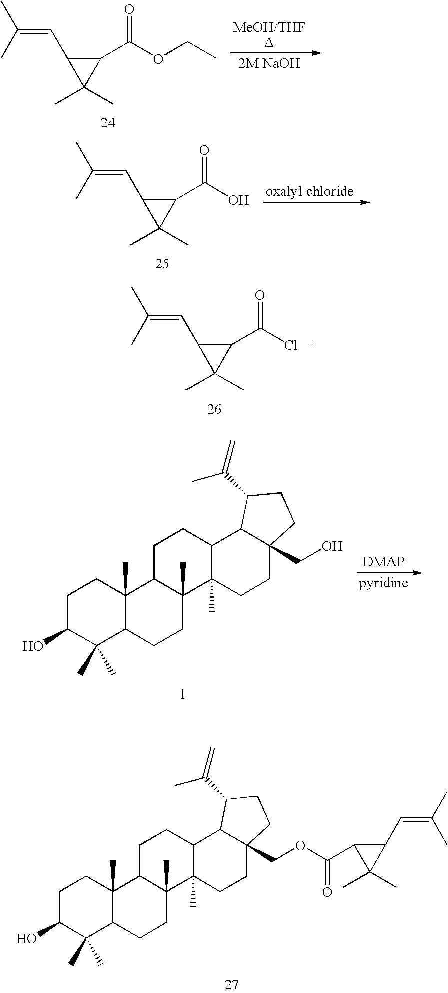

Ethyl chrysanthemate 24 (233 mmol) was mixed to a THF/MeOH solution (1:2) under inert atmosphere. 2 M NaOH solution (93 ml) was slowly added to the mixture, and then, the reaction mixture was heated in a bath at 80° C. for 4 hours until no starting material was present as determined by TLC (hexane:ethyl acetate 6:1, 5% by volume of acetic acid). The solvent was evaporated, the crude product obtained was dissolved in water (400 ml) and extracted with diethyl ether. The aqueous phase was acidified with hydrochloric acid, and diluted with diethyl ether. The ether phase was washed and the solvent was evaporated in vacuum, thus giving chrysanthemic acid 25 (yield: 90%).

Chrysanthemic acid 25 (5.9 mmol) dissolved in anhydrous dichloromethane (30 ml) was added with oxalyl chloride (11.8 mmol) at room temperature under inert atmosphere. After six hours, the solvent was evaporated and the evaporation residue was taken up in dry dichloromethane, and reevaporated. The procedure was repeated three times, thus giving chrysanthemic acid chloride 26 (yield: 81%).

Betulin 1 (0.9 mmol), chrysanthemic acid chloride 26 (1.1 mmol) and DMAP (0.9 mmol) were agitated in pyridine at 40° C. under inert atmosphere for 48 hours. EtOAc (100 ml) was added to the mixture, organic phase was washed with water, the solvent was evaporated, and the residue was recrystallized in cyclohexane. 28-chrysanthemate of betulin 27 was obtained with a yield of 63%.

### Example 9

### Preparation of 28-cinnamic Acid Ester of Betulin

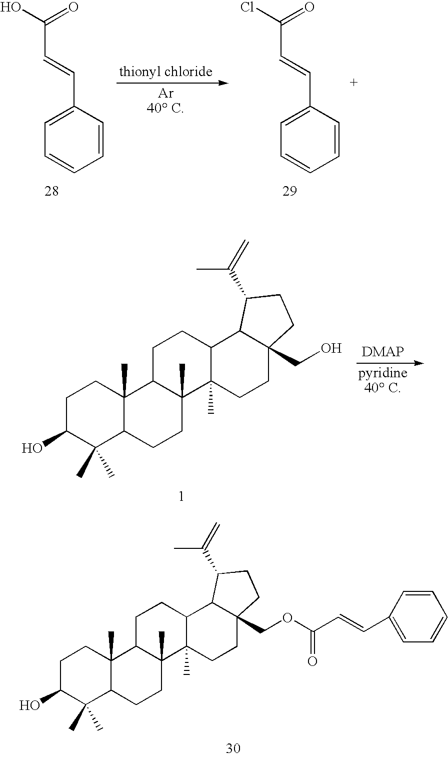

Cinnamic acid 28 (18.06 mmol) and thionyl chloride (180.6 mmol) wore mixed under inert argon atmosphere at 40° C. for 24 hours, Solvent was evaporated under vacuum, followed by dissolving the evaporation residue twice in dichloromethane and evaporation, thus giving cinnamic acid chloride 29 (yield: 99%).

Betulin 1 (5.4 mmol) and cinnamic acid chloride 29 (5.6 mmol) were agitated in dry pyridine (80 ml) in the presence of DMAP (5.6 mmol) under inert argon atmosphere at 40° C. for 24 hours. Toluene (100 ml) was added, and the organic phase was washed, Solvent was evaporated, followed by purification of the crude product by recrystallization in a cyclohexane/toluene solvent. 28-cinnamic acid ester of betulin 30 was obtained with a yield of 67%.

### Example 10

### Preparation of Fatty Acid Esters of Betulin

Betulin 1 (5 mmol) and fatty acid (5 mmol) were weighed in a flask equipped with a water separation tube. Toluene and catalytic amount of isopropyl titanate were added, followed by refluxing the reaction mixture in an oil bath for about 5 hours. The reaction mixture was allowed to cool to room temperature, the organic layer was washed with sodium hydrogen carbonate solution, separated, dried over sodium sulfate and the solvent was evaporated to dryness. The crude product obtained, betulin monoester, was purified by chromatography, if necessary. In case more than 2 equivalents of the fatty acid and 1 equivalent of betulin were used, also betulin diesters were obtained as products as shown in table 1. Table 1 shows the yields of the esterification reactions of betulin with fatty acids, and degrees of esterification.

TABLE 1

| | | | Total | Degree of C₃ | Degree of C₂₈ |
|---|---|---|---|---|---|
| | | Reflux | yield | esterification | esterification |
| Fatty acid | Catalyst | time (h) | (%) | (%) | (%) |
| Isostearic | Isopropyl | 3 | 81 | 0 | 40 |
| acid | titanate | | | | |
| Isostearic | p-toluene- | 4.5 | 99 | 10 | 95 |
| acid | sulfonic acid | | | | |
| Oleic acid | p-toluene- | 18.5 | 93 | 40 | 100 |
| | sulfonic acid | | | | |

### Example 11

### Preparation of 28-amide Derivatives of Betulin

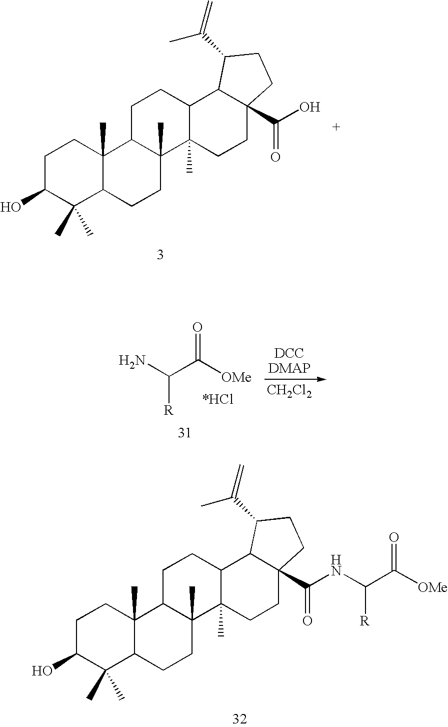

Betulinic acid 3 was prepared by oxidizing betulin 1 according to the U.S. Pat. No. 6,280,778. Betulinic acid 3 (5 mmol) and aminoacid methyl ester hydrochloride 31 (5 mmol) were weighed in a flask and dissolved in dichoromethane. The flask was purged with argon, dichloromethane (5 mmol) and DMAP (2.5 mmol) were added and mixing was continued for 20 hours. The reaction mixture was diluted with ethyl acetate, washed with water, dried over sodium sulfate, and the solvent was evaporated to dryness. The betulinic acid amide 32 crude product may be purified by chromatography, if necessary. Reaction conditions and crude yields of the products are shown in Table 2.

TABLE 2

| Amino acid | Reaction time (h) | Total yield (%) |
|---|---|---|
| L-aspartate dimethyl ester, HCl | 19 | >95 |
| L-histidine methyl ester, HCl | 18 | >95 |
| L-glutaminio acid methyl ester, HCl | 19 | >95 |
| L-lysine methyl ester, HCl | 19 | >95 |

### Example 12

### Preparation of 28-aspartateamide Dimethyl Ester of Betulonic Acid

Betulonic acid 2 (8.8 mmol) was dissolved in dichloromethane under inert atmosphere, followed by the addition of oxalyl chloride (18.6 mmol). The reaction mixture was agitated at room temperature for 20 hours. After completion of the reaction, the solvent was evaporated to dryness, the residue was again dissolved in dichloromethane, which was once more evaporated to dryness. The crude product obtained was washed with diethyl ether. The Yield was 7.5 mmol (85%) of betulonic acid chloride 33. Betulonic acid chloride 33 (4.2 mmol) and L-aspartic acid dimethyl ester hydrochloride 34 (5.5 mmol) were dissolved in dichloromethane, and triethyl amine (11 mmol) was added. The reaction mixture was agitated at room temperature for 20 hours. The reaction mixture was washed with diluted hydrochloric acid solution, water and dried over sodium sulfate. The solvent was evaporated to dryness, followed by purification of the crude product by chromatography, if necessary. Yield was 1.8 mmol (43%) of the 28-aspartateamide dimethyl ester of betulonic acid 35.

### Example 13

### Preparation of 28-N-acetylanthranilic Acid Ester of Betulin

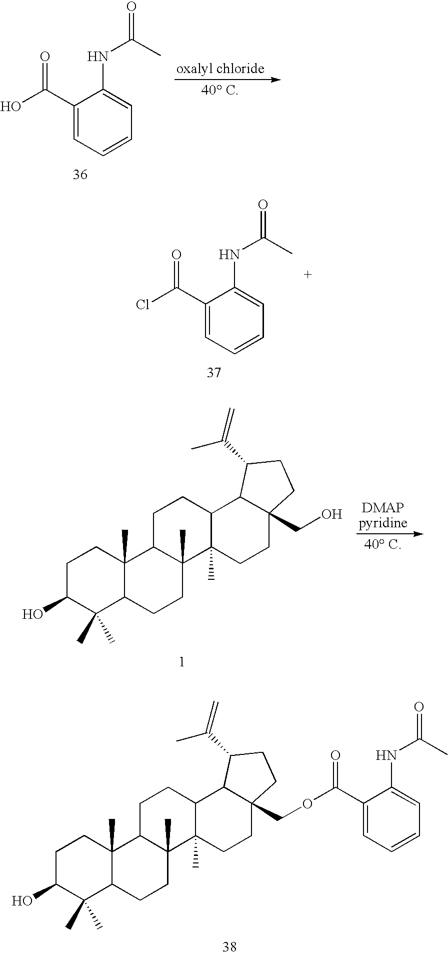

A mixture of N-acetylanthranilic acid 36 (25.0 mmol) and oxalyl chloride (250 mmol) was mixed for 16 hours at 40° C. Excessive oxalyl chloride was removed by evaporating the reaction mixture to dryness. The residue was twice dissolved in dichloromethane, which was evaporated to dryness. N-acetylanthranilic acid chloride 37 was thus obtained with a quantitative yield. A mixture of betulin 1 (11.29 mmol), DMAP (11.29 mmol), N-acetylanthranilic acid chloride 37 and pyridine (80 ml) was agitated for 24 hours at 40° C. After completion of the reaction, the reaction mixture was diluted with ethyl acetate and washed with diluted hydrochloric acid solution, and water and dried over sodium sulfate. The solvent was evaporated, followed by purification of the crude product by chromatography, thus giving 28-N-acetylanthranilic acid ester of betulin 38 with a yield of 25%.

### Example 14

### Preparation of 28-nicotinic Acid Ester of Betulin (Comparative)

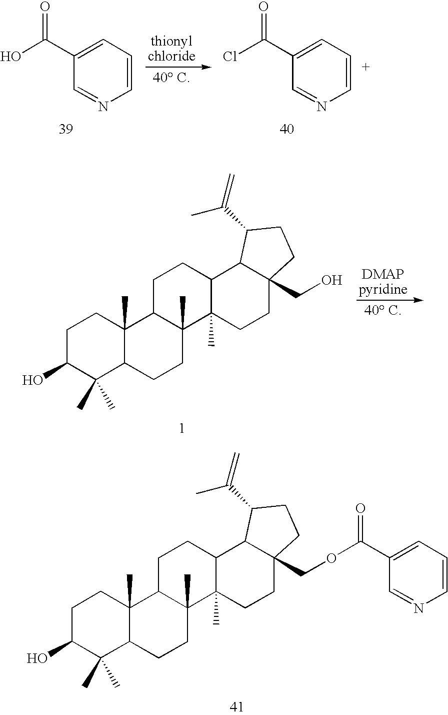

A mixture of nicotinic acid 39 (25.0 mmol) and thionyl chloride (250 mmol) was mixed for 24 hours at 40° C. Excessive thionyl chloride was removed by evaporating the reaction mixture to dryness. The residue was twice dissolved in dichloromethane, which was evaporated to dryness. Nicotinic acid chloride 40 was thus obtained. A mixture of betulin 1 (2.26 mmol), DMAP (126 mmol), nicotinic acid chloride 40 (2.71 mmol) and pyridine (10 ml) was agitated for 24 hours at 40° C. After completion of the reaction, the reaction mixture was diluted with ethyl acetate and washed with diluted hydrochloric acid solution, and water and dried over sodium sulfate. The solvent was evaporated, followed by purification of the crude product by recrystallization in cyclohexane, thus giving 28-nicotinic acid ester of betulin 41 with a yield of 88%.

### Example 15

### Preparation 3,28-diacetoxy-19,20-ene-29-succinic Anhydride of Betulin

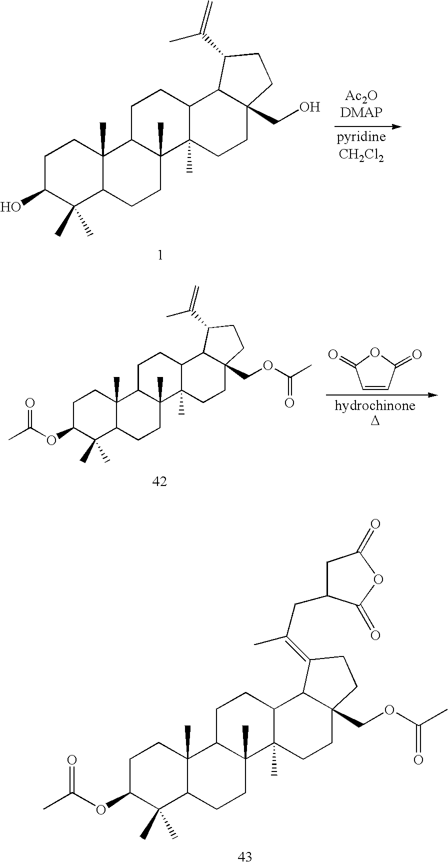

a) Acetic anhydride (19.2 ml, 203 mmol) is added to a mixture of betulin 1 (15.0 g, 33.88 mmol), DMAP (0.41 g, 3.39 mmol), pyridine (25 ml, 309 mmol), and dichloromethane (150 ml). The reaction mixture was agitated at room temperature for 17 hours. The organic phase was washed with 10% hydrochloric acid solution (200 ml), saturated NaHCO₃ solution (400 ml), water (100 ml), and dried over Na₂SO₄. The solvent was evaporated in vacuum, thus giving 3,28-diacetoxy betulin 42 with a yield of 97%.

b) A mixture of 3,28-diacetoxy betulin 42 (4.57 g, 8.68 mmol) and hydrochinone (96 mg, 0.87 mmol) was heated at 200° C., followed by the addition of succinic anhydride (2.50 g, 25.02 mmol) during 2 hours to the reaction flask. The reaction product, 3,28-diacetoxy-19,20-ene-29-succinic anhydride of betulin 43 was obtained with a yield of 100% (5.41 g, 8.65 mmol).

### Example 16

### Preparation of 3-deoxy-2,3-dihydrobetulin (Comparative)

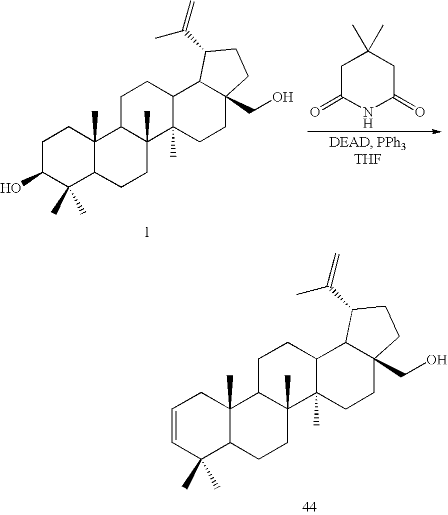

A solution of diethylazo dicarboxylate (DEAE, 20.71 ml, 45.18 mmol) in dry THF (100 ml) was added dropwise linden a nitrogen atmosphere to a mixture of betulin 1 (5.00 g, 11.29 mmol), triphenyl phosphine (PPh₃, 11.85 g, 45.18 mmol), and 3,3-dimethyl glutarimide (6.38 g, 45.18 mmol) in an ice bath. The reaction mixture was allowed to warm to room temperature, and agitating was continued for 24 hours. The precipitate formed was separated by filtering, followed by evaporating the solvent in vacuum. The crude product was purified by chromatography, thus giving 3-deoxy-2,3-dihydrobetulin 44 (1.47 g, 3.45 mmol, 31%),

### Example 17

### Preparation of 3-O-Diels-Alder Adduct of Betulin

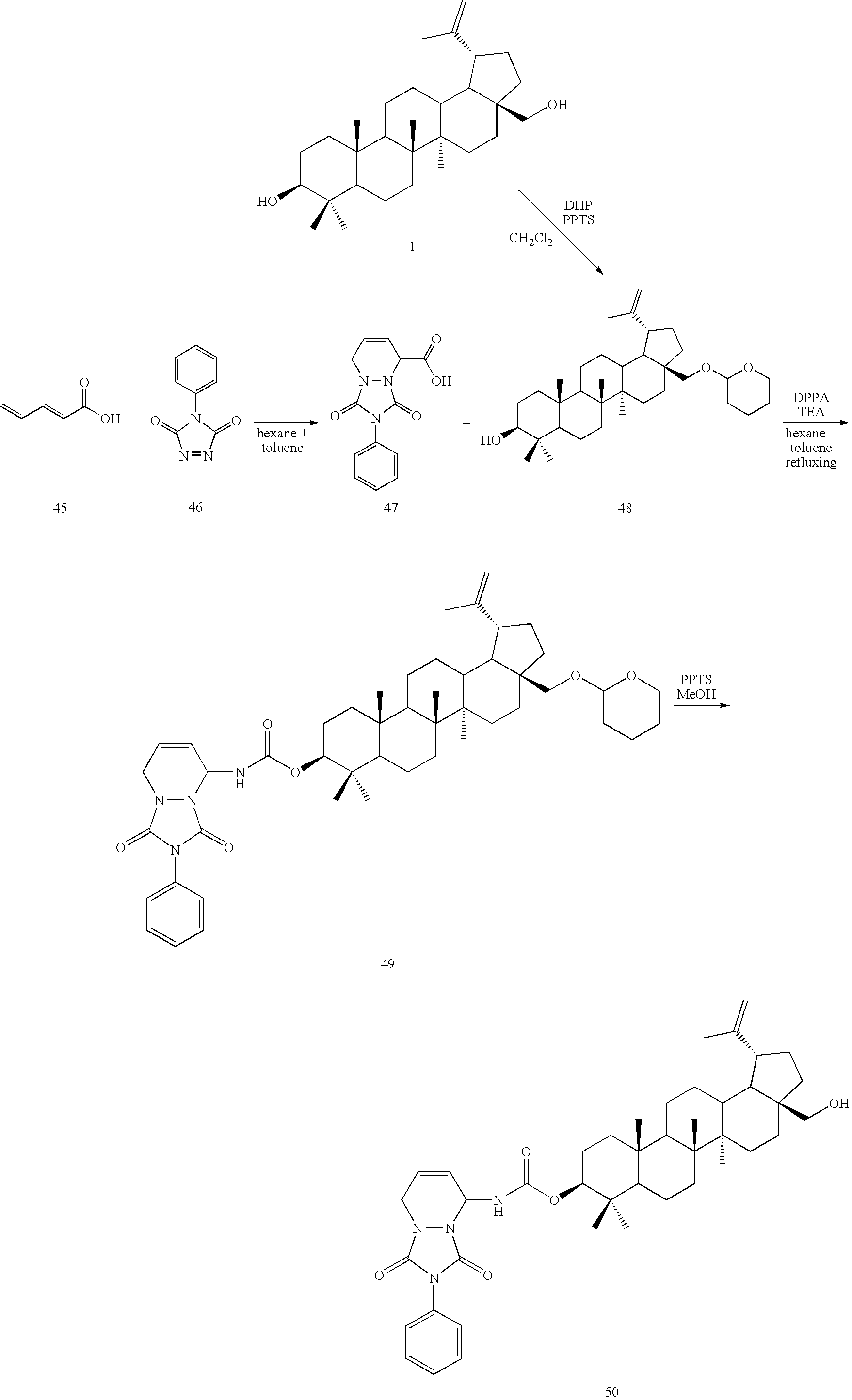

2,4-pentadiene acid 95 (196 mg, 2.0 mmol) and 4-phenyl-1,2,4-triazolin-3,5-dion 46 (350 mg, 2.0 mmol) were dissolved in a mixture of hexane and toluene. The reaction mixture was agitated under inert atmosphere at room temperature for 3 days. After completion of the reaction, the solvent was evaporated, thus giving the Diels-Alder adduct 47 (493 mg, 1.80 mmol, 90%).

Pyridinium-p-toluenesulfonate (PPTS) (0.68 g, 2.71 mmol) and dihydropyran (DHP) (2.09 g, 24.9 mmol) were added to betulin 1 (10.0 g, 22.6 mmol) in dichloromethane (330 ml) under inert atmosphere, and then the reaction mixture was agitated at room temperature for 5 days. After completion of the reaction, the organic phase was washed with saturated NaHCO₃ solution (150 ml) and water (150 ml), followed by drying over Na₂SO₄. The solvent was evaporated in vacuum, and the crude product obtained was purified by chromatography, thus giving the 28-tetrahydropyran ether of betulin 48 (3.46 g, 6.55 mmol, 29%).

28-tetrahydropyran ether of betulin 48 (116 mg, 0.22 mmol) and the Diels-Alder adduct 47 (60 mg, 0.22 mmol) were dissolved in a mixture of hexane and toluene. Diphenylphosphoryl azide (DPPA) and triethylamine (TEA) were added to the reaction mixture, which was refluxed for 24 hours. After completion of the reaction, the reaction mixture was diluted with ethyl acetate, the organic phase was washed with water, NaHCO₃ solution, diluted hydrochloric acid solution and water, followed by drying over Na₂SO₄. The solvent was evaporated in vacuum, thus giving crude product (419 mg) that was purified by chromatography, thus giving the 3-O-Diels-Alder adduct of the 28-tetrahydropyran ether of betulin 49 (yield: 50%).

A mixture of the 3-O-Diels-Alder adduct of the 28-tetrahydropyran ether of betulin 49 (50 mg, 0.063 mmol), pyridinium-p-toluene sulfonate (PPTS) (3 mg, 0.013 mmol), and methanol (10 ml) was agitated at room temperature under an inert atmosphere for two weeks. After completion of the reaction, NaHCO₃ solution (10 ml) was added to the reaction mixture. The aqueous phase was extracted with ethyl acetate (40 ml), which was washed with water (80 ml), dried over Na₂SO₄, followed by evaporation of the solvent in vacuum. The crude product was purified by chromatography. 3-O-Diels-Alder adduct of betulin 50 was thus obtained with a yield of about 50%.

### Example 18

### Preparation of the Diels-Alder-adduct of 4-methylurazole with Betulin

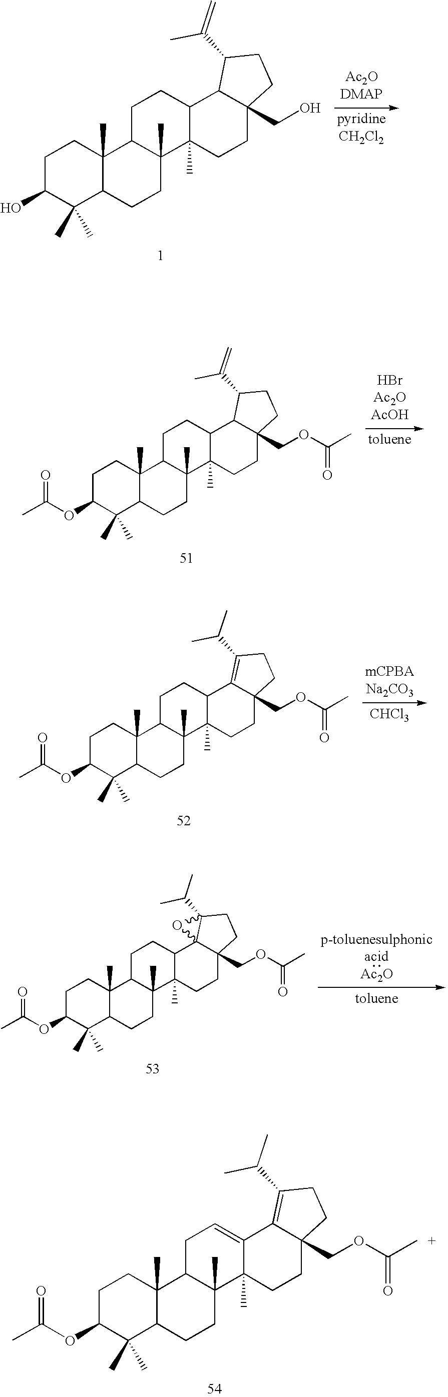

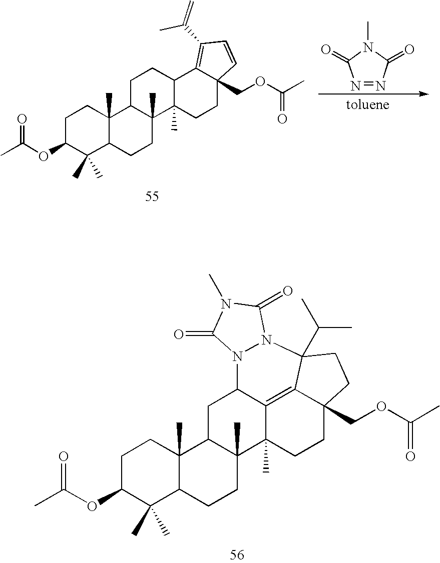

To a mixture of betulin 1 (15.0 g, 33.88 mmol), NAT-dimethylamino pyridine (DMAP, 0.41 g, 3.39 mmol), pyridine (25 ml, 309 mmol) and dichloromethane (150 ml) acetic anhydride (19.2 ml, 203 mmol) was added. The reaction mixture was mixed at RT for 17 hours. Organic phase was washed with 10% hydrochloric acid solution (200 ml), saturated NaHCO₃ solution (400 ml), and water (100 ml) and dried over Na₂SO₄. The solvent was evaporated in vacuum, thus giving betulin 3,28-diacetate 51 (yield: 97%).

To a mixture of hydrobromic acid (1-1Br) (47%, 250 g), acetic anhydride (100 g), and acetic acid (300 g), betulin 3,28-diacetate 51 (17.41 g, 33.05 mmol) dissolved in toluene (200 ml) was added. The mixture was allowed to stand at RT for 3 weeks. The reaction mixture was diluted with water (400 ml). The aqueous phase was separated and extracted with toluene (400 ml), Pooled organic phases were washed with water (30 ml), saturated NaHCO₃ solution (600 ml), dried over Na₂SO₄ and solvent was evaporated in vacuum. The crude product was purified by chromatography giving 3β,28-diacetoxylup-18-ene 52 (7.36 g, 13.97 mmol, 42%).

To a mixture of 3β,28-diacetoxylup-18-ene 52 (4.91 g, 9.33 mmol) and Na₂CO₃ (4.94 g, 46.65 mmol) in chloroform (120 ml), m-chloroperbenzoic acid (mCPBA, 3.69 g, 14.92 mmol) was added, followed by mixing of the mixture at RT for 2 hours. The organic phase was washed with water (150 ml), saturated NaHSO₃ solution (150 ml), dried over Na₂SO₄, and the solvent was evaporated in vacuum. The crude product was recrystallized in ethanol, thus giving 3β,28-diacetoxylup-18ξ,19ξ-epoxylupane 53 (3.31 g, 6.09 lima, 65%).

3β,28-diacetoxylup-18ξ,19ξ-epoxylupane 53 (2.00 g, 3.68 mmol) and p-toluenesulfonic acid (0.42 g, 2.21 mmol) were dissolved in toluene (80 ml), and then acetic anhydride (0.56 ml, 5.90 mmol) was added. The reaction mixture was refluxed for four hours. Organic phase was washed with saturated NaHCO₃ solution (150 ml), and water (100 ml), dried over Na₂SO₄, and the solvent was evaporated in vacuum. The crude product was purified by chromatography and crystallized in ethanol, thus giving a mixture of 3β,28-diacetoxylupa-12,18-diene 54 and 3β,28-diacetoxylupa-18,21-diene 55 (4:1) (1.31 g, 2.50 mmol, 68%).

3β,28-diacetoxylupa-12,18-diene 54, 3β,28-diacetoxylupa-18,21-diene 55 (total amount of 100 mg, 0.19 mmol), and 4-methyl-1,2,4-triazolin-3,5-dion (32 mg, 0.29 mmol) were dissolved in toluene (5 ml), and then the reaction mixture was agitated at room temperature for 24 hours. The solvent was evaporated in vacuum and the crude product was purified by chromatography, thus giving Diels-Alder-adduct of 4-methylurazole with betulin 56 (60 mg, 0.09 mmol, 49%).

### Example 19

### Preparation of Dials-Alder Adduct of p-acetyl-4-phenylurazole with Betulin

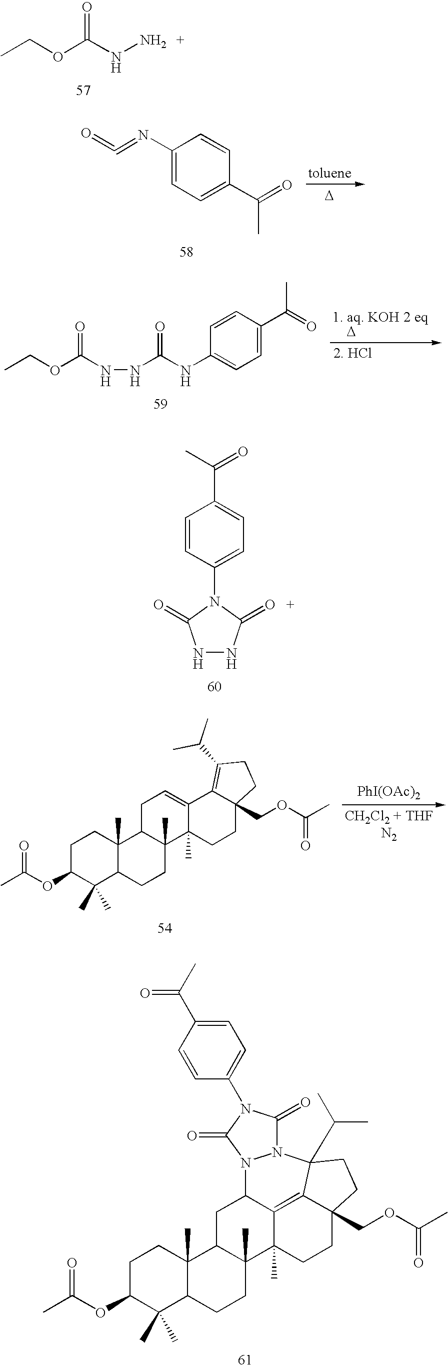

To ethylhydrazin 57 (2.64 mmol) in toluene (5 ml), 4-acetylphenyl isocyanate 58 (2.64 mmol) dissolved in 5 ml of toluene was added dropwise under an inert atmosphere. Agitation was continued for 2 hours at room temperature, and at 80° C. for 2 hours. Filtering of the precipitate formed and drying thereof in the oven gave p-acetyl-4-phenyl-1-carbethoxy semicarbazide 59 (yield; 90%).

This p-acetyl-4-phenyl-1-carbethoxy semicarbazide 59 (1.13 mmol) was heated at 70° C. in an aqueous 4M KOH solution (2.26 mmol) for 1.5 hours. The precipitate was filtered off, followed by acidification of the cooled filtrate with concentrated HCL solution. The precipitate formed was filtered and dried in a desiccator, thus giving p-acetyl-4-phenylurazole 60 (yield: 65%).

A mixture of p-acetyl-4-phenylurazole 60 (50 mg, 0.229 mmol), and iodobenzene diacetate ((Phi(OAc)₂, 74 mg, 0.229 mmol) was agitated under Ar gas in an anhydrous THF:CH₂Cl₂ mixture (4 ml, 1:1) for 15 minutes yielding a red colour. 3β,28-diacetoxylupa-12,18-diene 54 (100 mg, 0.191 mmol) was dissolved in a THF:CH₂Cl₂ mixture (4 ml, 1:1) and added to the reaction flask, and agitation was continued for 24 hours at room temperature. The solvent was evaporated in vacuum. Purification of the crude product by chromatography gave a Diels-Alder adduct od betulin with p-acetyl-4-phenylurazole 61 (yield: 30%). Table 3 below shows the percent yields of the Diels-Alder adducts of betulin with urazole for different groups R:

TABLE 3

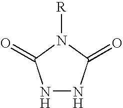

| R | Yield (%) | R | Yield (%) |
|---|---|---|---|
| 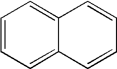 | 53 | 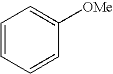 | 47 |
| H | 40 | 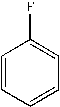 | 44 |
| 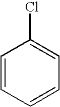 | 74 | 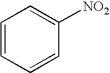 | 60 |
| 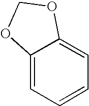 | 51 | 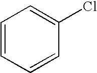 | 38 |
| 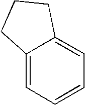 | 53 | 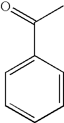 | 30 |
| 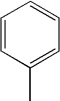 | 62 | | |

### Example 20

### Preparation of Betulin 3-acetoxy-28-1′,2′,3′-triazoles and Betulin 3-acetoxy-28-tetrazoles

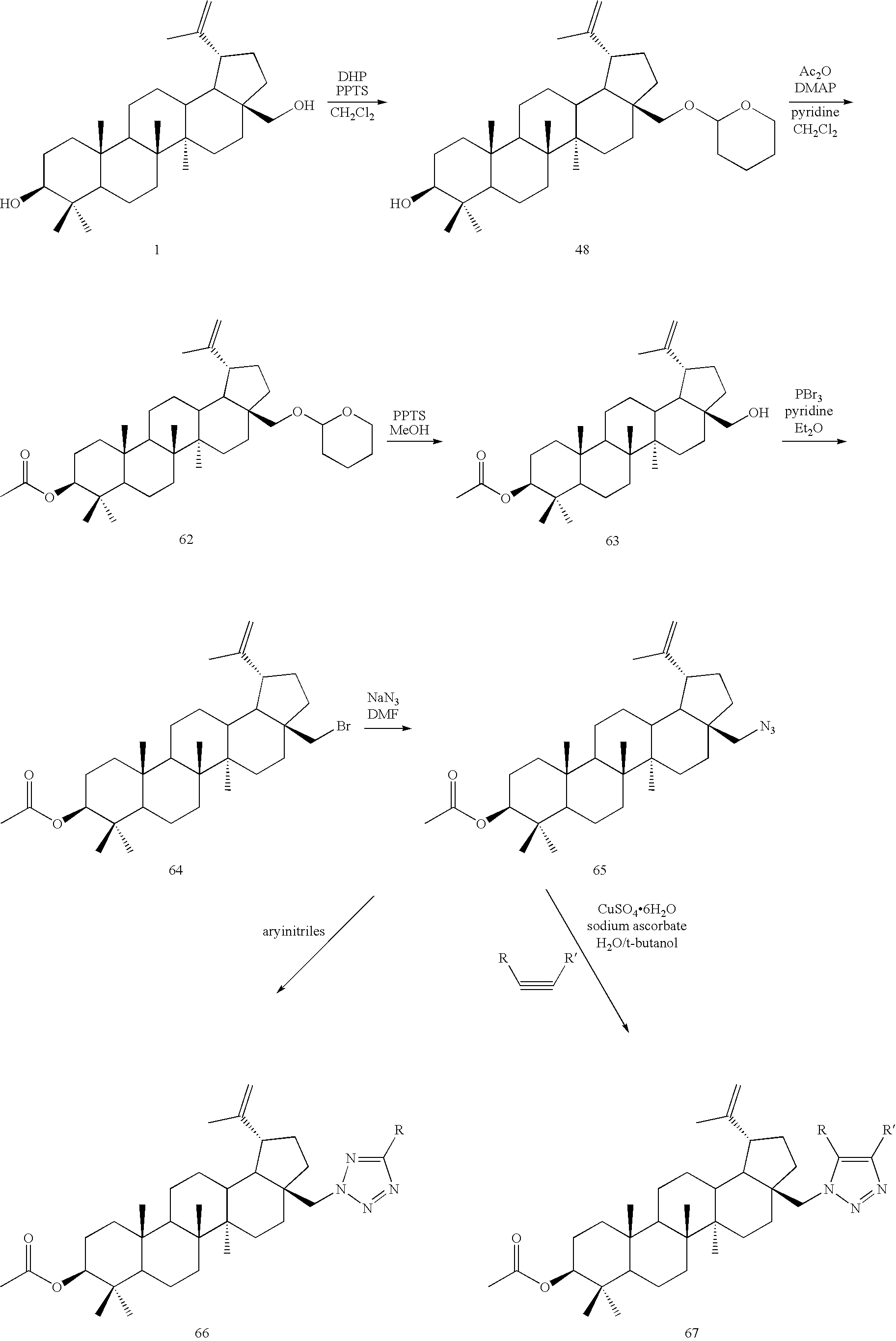

To betulin 1 (10.0 g, 22.6 mmol) in dichloromethane (330 ml), pyridinium-ptoluenesulfonate (PPTS) (0.68 g, 2.71 mmol), and dihydropyrane (DHP) (2.09 g, 24.9 mmol) were added under inert atmosphere, followed by agitation of the reaction mixture at room temperature for 5 days. After completion of the reaction, the organic phase was washed with saturated NaHCO₃ solution (150 ml) and water (150 ml), then dried over Na₂SO₄. The solvent was evaporated in vacuum, and then the crude product was purified by chromatography, thus giving betulin 28-tetrahydropyrane ether 48 (3.46 g, 6.55 mmol, 29%).

To a mixture of betulin 28-tetrahydropyrane ether 48 (5.00 g, 9.49 mmol), N,N dimethylamino pyridine (DMAP, 0.12 g, 0.95 mmol), pyridine (10 ml, 124 mmol) and dichloromethane (50 ml), acetic anhydride (5.4 ml, 57 mmol) was added. The reaction mixture was agitated at RT for 20 hours. The organic phase was washed with 10% hydrochloric acid solution (300 ml), saturated NaHCO₃ solution (400 ml), water (100 ml), and dried over Na₂SO₄. The solvent was evaporated in vacuum, thus giving betulin 3-acetoxy-28-tetrahydropyrane ether 62 (yield: 95%).

A mixture of betulin 3-acetoxy-28-tetrahydropyrane ether 62 (3.00 g, 5.27 mmol), pyridinium-p-toluenesulfonate (PPTS) (226 mg, 1.06 mmol), and methanol (100 ml) was agitated at room temperature under an inert atmosphere for 2 weeks. After completion of the reaction, NaHCO₃ solution (100 ml) was added to the reaction mixture. The aqueous phase was extracted with ethyl acetate (400 ml), followed by washing with water (800 ml), dried over Na₂SO₄, the solvent was evaporated in vacuum, thus giving betulin 3-acetate 63 (yield: 94%).

To a mixture of betulin 3-acetate 63 (100 mg, 0.21 mmol) and diethyl ether (10 ml), pyridine (163 mg, 2.1 mmol) and phosphorus tribromide (PBr₃) (280 mg, 1.9 mmol) were added at −5° C. under an inert atmosphere. The reaction mixture was allowed to warm to room temperature while continuing mixing for 24 hours. After completion of the reaction, the organic phase was washed with water (100 ml), NaHCO₃ solution (80 ml) and dried over Na₂SO₄. The solvent was evaporated in vacuum, thus giving betulin 3-acetoxy-28-bromide 64 (yield: 63%).

A mixture of betulin 3-acetoxy-28-bromide 64 (200 mg, 0.36 mmol), NaN₃ (230 mg, 3.6 mmol), and DMF (20 ml) was heated at 100° C. under an inert atmosphere for 24 hours. After completion of the reaction, the solvent was evaporated in vacuum and the residue was taken up in ethyl acetate (100 ml). The organic phase was washed with water (225 ml), dried over Na₂SO₄ and the solvent was evaporated in vacuum, thus giving 149 mg of the crude product comprising 20% of betulin 3-acetoxy-28-azide 65.

Using known methods, betulin 3-acetoxy-28-azide 65 may be reacted with arylnitriles, giving betulin 3-acetoxy-28-tetrazoles 66, or with a functional alkyne in the presence of CuSO₄.5H₂O and sodium ascorbate in an aqueous butanol solution, giving betulin 3-acetoxy-28-1′,2′,3′-triazoles 67.

### Example 21

### Preparation of Betulin 3,28-dibetaine Ester

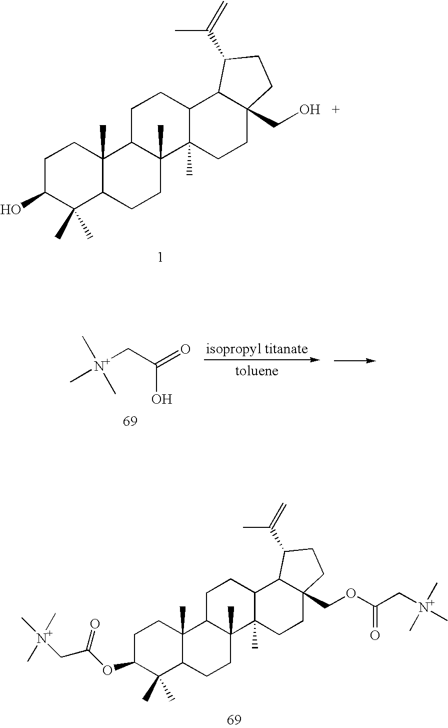

Betulin 1 (7.0 g, 16 mmol) and betaine 68 (3.8 g, 32 mmol) were dissolved in toluene (150 while heating. Thereafter, isopropyl titanate Ti(OCHMe₂)₄ catalyst (0.85 g, 3 mmol) was added, and the mixture was refluxed for 3 hours. The solid final product was separated by filtration. Tetrahydrofurane was added to remove by-products, and filtering was repeated, Yield of the final product 69 (betulin 3,28-dibetaine ester) was 2.7 g (4.1 mmol, 26%).

### Example 22

### Preparation of 28-acetate of Betulonic Alcohol

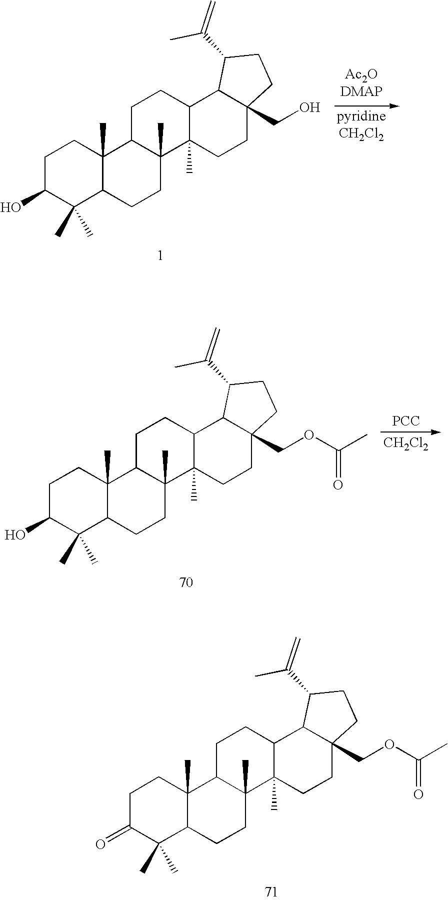

a) To a mixture of betulin 1 (8.00 g, 18.1 mmol) and 4-dimethylamino pyridine (DMAP) (0.8 g, 6.55 mmol) in dichloromethane (72 ml), pyridine (72 m) and acetic anhydride (1.8 ml, 19.1 mmol) were added and the reaction mixture was agitated at RT for 22 hours. The organic layer was washed with 10% hydrochloric acid solution, water, saturated NaHCO₃ solution, and dried over Na₂SO₄. The solvent was evaporated in vacuum, followed by purification of the crude product obtained by chromatography, thus giving 28-acetoxybetulin 70 (3.80 g, 45%).

b) A mixture of betulin 28-acetate (590 mg, 1.23 mmol) and pyridinium chlorochromate (PCC) (1.32 g, 3.14 mmol) in dichloromethane (60 ml) was agitated at RT (=room temperature) for 24 hours. The reaction mixture was diluted with diethyl ether (30 ml), agitated for 10 minutes, and the precipitate was filtered off. The filtrate was evaporated in vacuum and the crude product was purified by chromatography, thus giving 28-acetate of betulonic alcohol 71 (330 mg, 57%).

### Example 23

### Preparation of Betulonic and Betulinic Acids (Comparative)

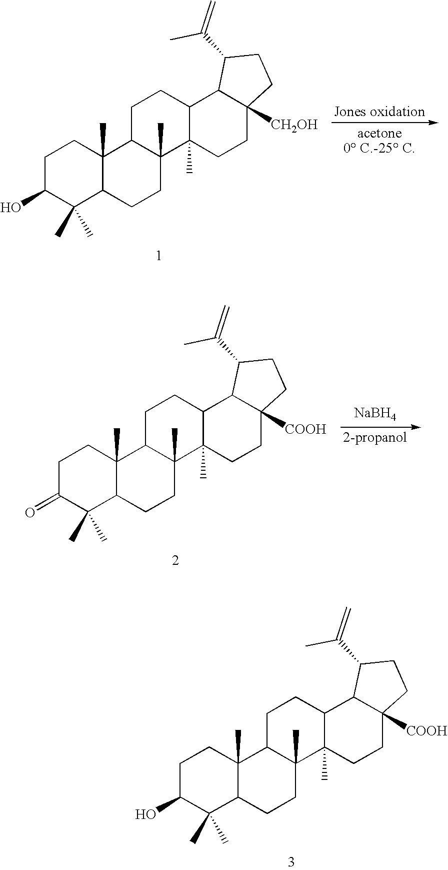

a) To a solution of betulin 1 (50 g, 113 mmol) in acetone (1500 ml), Jones reagent was added during 1 hour in an ice bath. The reaction mixture was allowed to warm to RT and agitation was continued for 21 hours. Methanol (700 ml) and water (1000 ml) were added to the reaction mixture. The precipitate was filtered, dried in vacuum, taken up in diethyl ether (600 ml) and washed with water, 7.5% hydrochloric acid, water, saturated NaHCO₃ solution, and with water. Half of the diethyl ether was evaporated in vacuum and the residue was treated with 10% NaOH solution. The precipitate was filtered, dried in vacuum, and dissolved in boiling methanol, followed by the addition of acetic acid (10 ml). The product was precipitated with water, filtered and dried in vacuum, thus giving betulonic acid 2 (22.3 g, 44%).

b) To betulonic acid 2 (10 g, 22 mmol) in 2-propanol (400 ml), NaBH₄ (1.76 g, 44.2 mmol) was added, and the reaction mixture was agitated at room temperature for 2 hours, 10% hydrochloric acid solution (600 ml) was added, the precipitate was filtered, washed with water and dried in vacuum. The crude product obtained was crystallized in ethanol, thus giving betulinic acid 3 (8.25 g, 18 mmol).

### Example 24

### Preparation of Betulonic Aldehyde (Comparative)

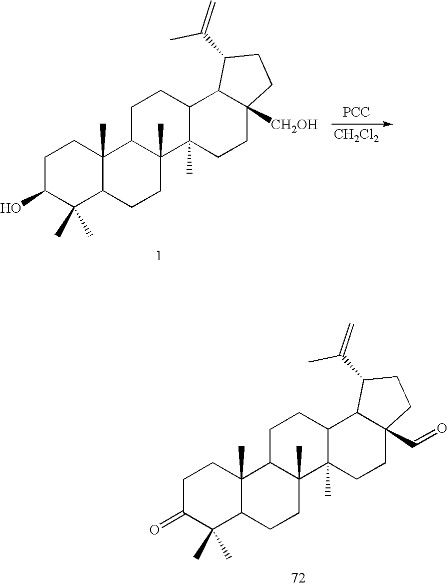

A mixture of betulin 1 (3.0 g, 6.8 mmol), pyridinium chlorochromate (PCC) (8.8 g, 41 mmol) and dichloromethane was agitated at room temperature for 1 hour. The reaction mixture was dissolved with diethyl ether and filtered through alumina. The filtrate was washed with water, 5% hydrochloric acid, again with water and dried over Na₂SO₄. The solvent was evaporated in vacuum and the crude product was crystallized in a mixture of hexane and ethyl acetate, thus giving betulonic aldehyde 72 (2.4 g, 82%).

### Example 25

### Preparation of 28-methyl Ester of Betulinic Acid

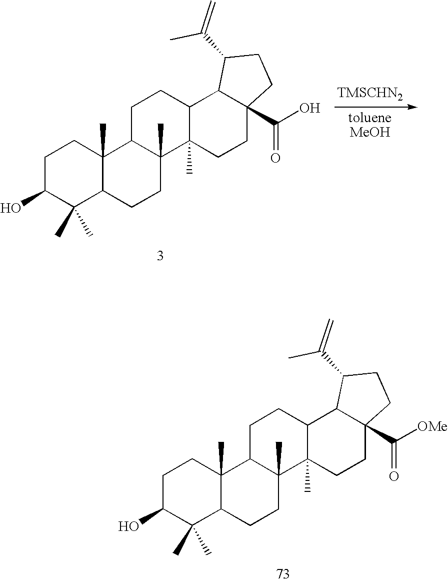

To a mixture of betulinic acid 3 (100 mg, 0.22 mmol), methanol (1 ml) and toluene (1.5 ml), a 2M solution of trimethylsilyl diazomethane in diethyl ether (0.17 ml, 0.33 ml) was added and the reaction mixture was agitated at room temperature for 40 minutes. The solvent was evaporated in vacuum, thus giving 28-methyl ester of betulinic acid 73 (68 mg, 66%).

### Example 26

### Preparation of Betulin Aldehyde, Betulin 28-oxime and Betulin 3,28-dioxime

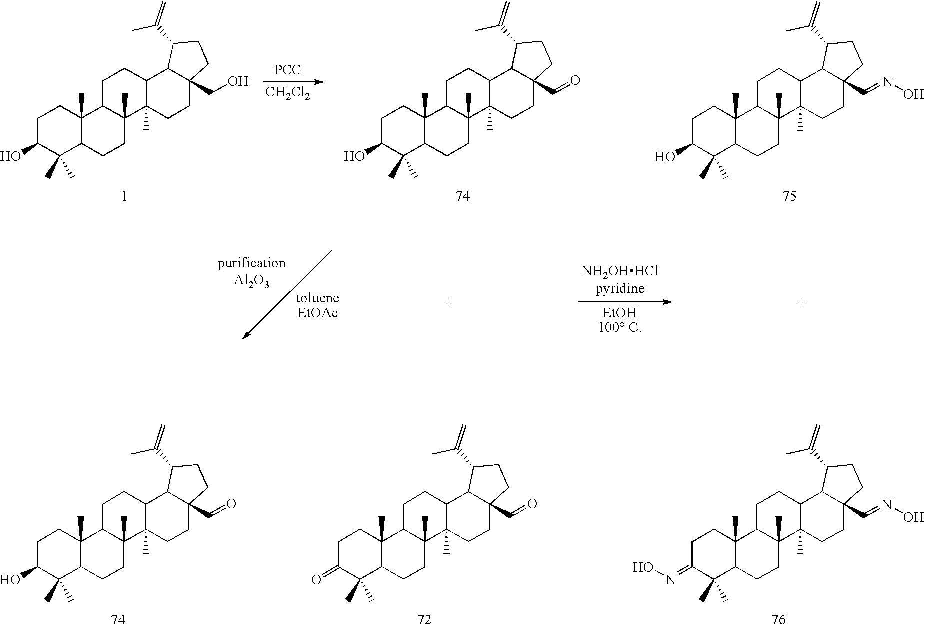

a) A mixture of betulin 1 (8.0 g, 18 mmol) and pyridinium chlorochromate (FCC) (7.0 g, 33 mmol) in dichloromethane (800 ml) was agitated at room temperature for 40 minutes. The reaction mixture was diluted with diethyl ether (200 ml) and filtered through alumina. The solvent was evaporated in vacuum and the crude product was purified by chromatography, thus giving betulin aldehyde 74 (0.36 g, 18%).

b) To a mixture of betulonic aldehyde 72, betulinic aldehyde 74, pyridine (40 ml) and ethanol (120 ml), hydroxylamine hydrochloride (10 g, 144 mmol) was added, followed by refluxing the reaction mixture for 18 hours. The solvent was evaporated in vacuum and the mixture of betulin 28-oxime 75 and betulin 3,28-dioxime 76 obtained was purified by chromatography, thus giving betulin 28-oxime 75 (0.97 g, 2.1 mmol) and betulin 3,28-dioxime 76 (0.32 g, 0.7 mmol),

### Example 27

### Preparation of Betulonic Alcohol

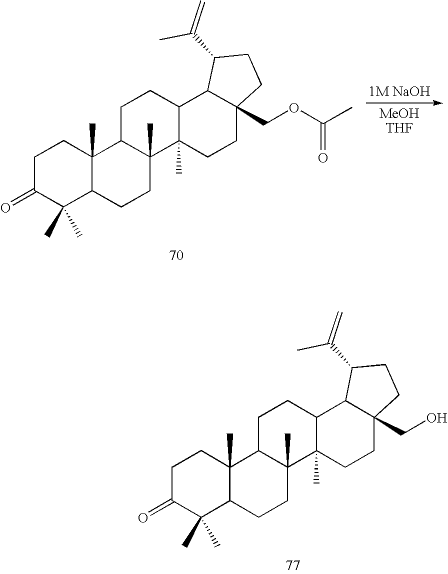

A mixture of betulonic 28-acetate 70 (15 mg, 0.032 mmol), methanol (0.3 ml), tetrahydrofurane (0.45 ml) and 1 M NaOH solution (0.16 ml) was agitated at RT for 20 hours. Water (4 ml) was added and the reaction mixture was made acidic with diluted hydrochloric acid. The aqueous phase was extracted with ethyl acetate, which was dried over Na₂SO₄ and evaporated in vacuum, thus giving 77 (7.0 mg, 50%).

### Example 28

### Preparation Betulin 3-acetoxyoxime-28-nitrile

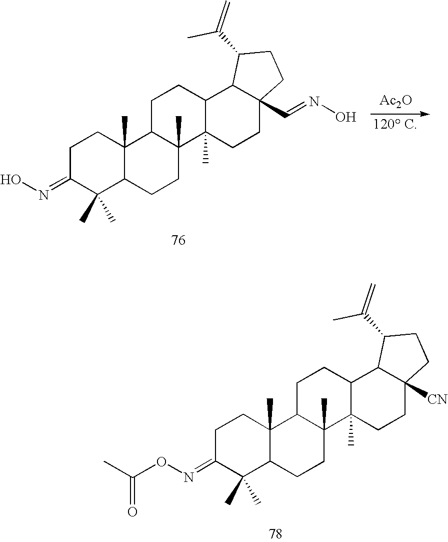

A mixture of betulin 3,28 dioxime 76 (100 mg, 0.2 mmol) and acetic anhydride (2.5 ml) was agitated at 120° C. for 2 hours. The reaction mixture was diluted with water and the precipitate was filtered off. The precipitate was taken up in chloroform, washed with water, saturated NaHCO₃ solution, water and dried over Na₂SO₄. The solvent was evaporated in vacuum and the crude product was purified by chromatography, thus giving betulin 3-acetoxyoxime-28-nitrile 78 (37 mg, 34%).

### Example 29

### Preparation of Betulin 28-acetic Acid Methyl Ester

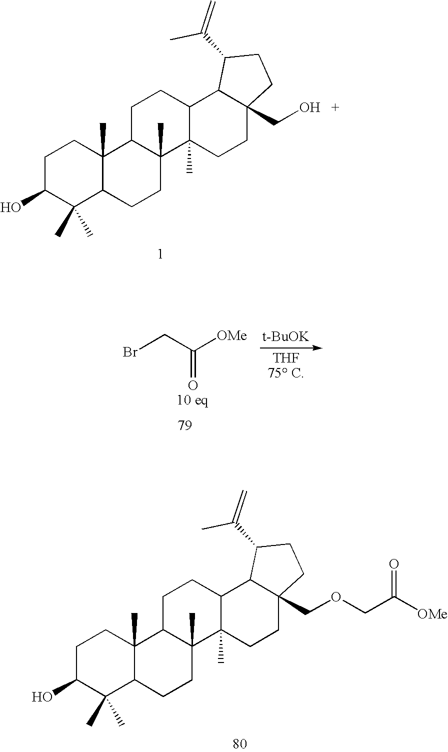

A mixture of betulin 1 (1.0 g, 2.3 mmol) and potassium ter-butoxide (2.5 g, 23 mmol) in tetrahydrofurane (50 ml) was agitated at 75° C., followed by the addition of methylbromoacetate 79 (2.1 ml, 23 mmol). The reaction mixture was agitated for 10 minutes, allowed to cool and then diluted with water. The precipitate was filtered and the crude product was purified by chromatography, thus giving betulin 28-acetic acid methyl ester 80 (0.2 g, 15%).

### Example 30

### Preparation of 20,29-dihydrobetulin and 20,29-dihydrobetulonic Acid

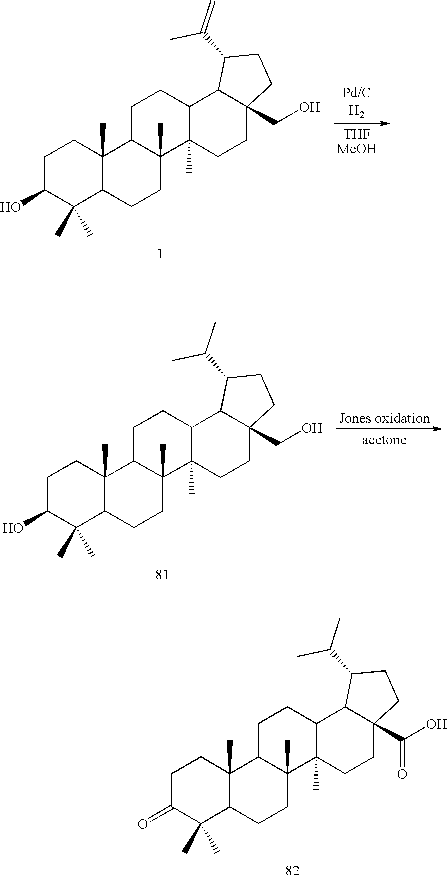

a) To a mixture of betulin 1 (2.0 g, 4.5 mmol), tetrahydrofurane (40 ml) and methanol (80 ml), 5% Pd/C (0.2 g) was added, followed by agitating the reaction mixture under hydrogen atmosphere for 22 hours. The reaction mixture was filtered, and the filtrate was evaporated in vacuum, thus giving 20,29-dihydrobetulin 81 (2.0 g, 99%).

b) To a mixture of 20,29-dihydrobetulin 81 (1.0 g, 2.3 mmol) and acetone (75 ml), Jones reagent was added. The reaction mixture was agitated for 20 hours, Methanol (20 ml) and water (40 ml) were added to the reaction mixture. The organic solvent was evaporated in vacuum and the aqueous phase was extracted with ethyl acetate, which was washed with water and dried over Na₂SO₄. The solvent was evaporated in vacuum and the crude product was purified by chromatography, thus giving 20,29-dihydrobetulonic acid 82 (320 mg, 31%).

### Example 31

### Preparation of a Diels-Alder Adduct of 4-methylurazole

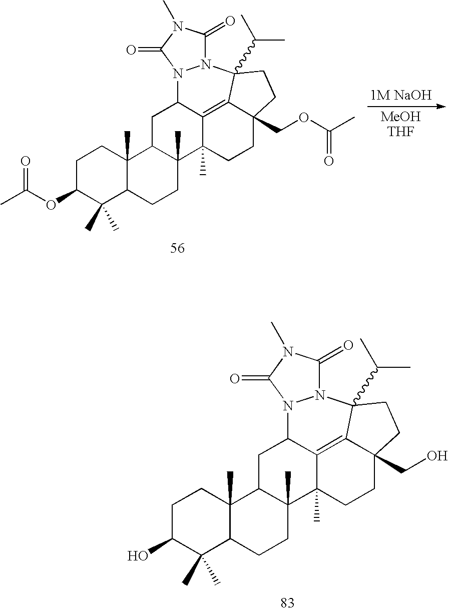

A mixture of Diels-Alder adduct of 4-methylurazole 56 (50 mg, 0.07 mmol), methanol (0.5 ml), tetrahydrofurane (0.8 ml) and 1 M aqueous NaOH solution (0.3 ml) was agitated at room temperature for 20 hours. The product was precipitated with water, the precipitate was filtered and dried, thus giving the Diels-Alder adduct of 4-methylurazole 83 (40 mg, 91%).

### Example 32

### Activity of the Betulin Derivatives Against Leishmaniasis

Leishmania donovani MHOM/SD/1962/1S-012D and L. tropica MHOM/IS/1990/LRC-L590 protozoa were used in the tests.

Promastigotes were grown in a broth containing Medium-199 (Sigma, St. Louis, Mo.) supplemented with 2 mM L-glutamine, 100 μM adenosine, 23 μM folio acid, antibiotics (100 IU penicillin G and 100 μg/ml streptomycin), 1×BME vitamin mixture, 25 mM 2-(N-morpholin)ethanesulfonic acid (MES), 4.2 mM NaHCO₃ and heat treated fetal calf serum (FCS, 10% v/v), pH being adjusted to 6.8, Promastigotes were grown at 26° C.

Axenic amastigotes of L. donovani were grown according to the procedure presented by Debrabant et al., 2004, in the RPMI 1640 broth containing 20% v/v of fetal calf serum (FCS), at 37° C., pH 5.5, Axenic amastigotes of L. tropica were grown as described for L. donovani with the exception that only 10% v/v FCS was used, at 36° C. without CO₂.

### Optimization of the Alamar Blue Determination and Testing of the Compounds Pounds

Dilutions of promastigotes and axenic amastigotes of L. donovani and L. tropica in a cultivation broth were prepared to obtain concentrations in the range of 1.6×10⁷ to 4.2×10² parasites/ml. Each dilution with the desired concentration of the parasite was dispensed as three parallel samples (250 μl/well) to a 96 well microtiter plate having a flat bottom (NUNC, Denmark), followed by the addition of Alamar Blue reagent (25 μl/well, ENCO). The plates were read (λ_{cm}=544 nm; λₑₓ2=590 nm) after following incubation times (3, 9, 24, 48 and 72 h) using a fluorescence microtiter plate reader (Fluoroskan, Ascent Fla., Finland).

Activity of the compounds was tested both for L. donovani and L. tropica species. Promastigotes (2×10⁶ cells/ml) or axenic amastigotes (5.0×10⁵ cells/ml) were introduced as three parallel samples (125 μl/well) to 96 well microtiter plates having flat bottoms, the wells containing each compound diluted in the cultivation broth (125 with final concentration of DMSO of 1%), Initially, the concentration of the compounds was 50 mM. Amphotericin B (1 μM) was included as positive control (Sigma, St. Louis, Mo.), Broth containing DMSO was used as negative control. The parasites were tested either with or without the compounds by incubating at 26° C. (promastigotes), at 36° C. (axenic amastogotes of L. tropica) or at 37° C. (axenic amastogotes of L. donovani). After 24 hours, the Alamar Blye reagent (25 μl/well) was added, the plates were still incubater for 24 hours, followed by fluorescence determination. GI₅₀ assay was carried out using the same procedure, the concentrations of the compounds to be tested ranging, however, between 50 and 0.01 μM. The results are presented in the following table 4.

TABLE 4

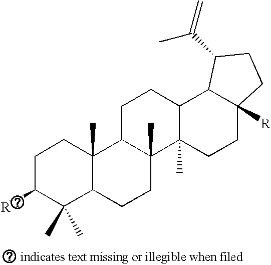

| | Inhibition of |
|---|---|
| Compound | Leishmania (%) |
| 28-acetate of betulonic alcohol | 40.6 |
| 28-methylester of betulonic acid | 40.1 |
| betulinic aldehyde | 65.0 |
| betulin 3,28-dioxime | 72.4 |
| betulin 28-oxime | 66.8 |
| betulonic alcohol | 44.0 |
| betulin 3-acetoxyoxime-28-nitrile | 66.4 |
| betulin 28-acetic acid methylester | 95.3 |
| 20,29-hydrobetulonic acid | 73.4 |
| betulin | 35.0 |
| 2,3-didehydro-3-deoxybetulin | 13.2 |
| betulonic acid | 97.6 |
| betulinic acid | 39.8 |
| 28-aspartateamide dimethylester of betulonic acid | 69.3 |
| betulonic aldehyde | 46.2 |
| betulin 28-N-acetylanthranilic acid ester | 59.2 |
| betulin 28-chrysanthemate | 13.4 |
| betulin 28-carboxymethoxy mentholester | 16.6 |
| positive control: amphotericine B (1 μM) | 55.4 |
| negative control: broth + DMSO | 0.0 |

### Example 33

### Activity of Diels-Alder-Derivatives of Betulin Against Leishmaniasis

Testing of the compounds was carried out as described in example 32. The results are presented in the table 5 below.

TABLE 5

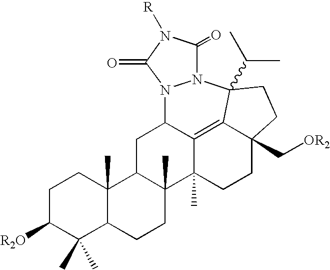

| | | | Inhibition of | GI₅₀ |
|---|---|---|---|---|
| Compound | R | R2 | Leishmania (%) | (μM) |
| Diels-Alder adduct of 3β,28-diacetoxylupa-12,18- | H | Ac | 87.6 | 25.5 |
| diene and urazole | | | | |
| Diels-Alder adduct of 3β,28-diacetoxylupa-12,18- | Me | Ac | 98.2 | 8.9 |
| diene and 4-methylurazole | | | | |
| Diels-Alder adduct of lupa-12,18-diene and 4- | Me | H | 50.0 | |
| methylurazole | | | | |
| Diels-Alder adduct of 3β,28-diacetoxylupa-12,18- | Ph | Ac | 36.2 | |
| diene and 4-phenylurazole | | | | |
| Diels-Alder adduct of lupa-12,18-diene and 4- | Ph | H | 47.5 | |
| phenylurazole | | | | |
| Diels-Alder adduct of 3β,28-diacetoxylupa-12,18- | PhCH₂ | Ac | 24.7 | |
| diene and 4-benzoylurazole | | | | |
| Diels-Alder adduct of 3β,28-diacetoxylupa-12,18- | 4-F-Ph | Ac | 47.8 | |
| diene and p-fluoro-4-phenylurazole | | | | |
| Dials-Alder adduct of 3β,28-diacetoxylupa-12,18- | 4-Cl-Ph | Ac | 27.3 | |
| diene and p-chloro-4-phenylurazole | | | | |
| Diels-Alder adduct of 3β,28-diacetoxylupa-12,18- | 3-Cl-Ph | Ac | 29.7 | |
| diene and m-chloro-4-phenylurazole | | | | |
| Diels-Alder adduct of 3β,28-diacetoxylupa-12,18- | 3-MeO-Ph | Ac | 43.5 | |
| diene and m-methoxy-4-phenylurazole | | | | |
| Diels-Alder adduct of 3β,28-diacetoxylupa-12,18- | 3-NO₂-Ph | Ac | 29.5 | |
| diene and m-nitroxy-4-phenylurazole | | | | |
| Diels-Alder adduct of 3β,28-diacetoxylupa-12,18- | 4-Ac-Ph | Ac | 44.7 | |
| diene and p-acetoxy-4-phenylurazole | | | | |
| Diels-Alder adduct of 3β,28-diacetoxylupa-12,18- | Indan-5-yl | Ac | 22.5 | |
| diene and indan-5-yl urazole | | | | |
| Diels-Alder adduct of 3β,28-diacetoxylupa-12,18- | 1-naphthyl | Ac | 57.7 | |
| diene and 1-naphthylurazole | | | | |
| Diels-Alder adduct of 3β,28-diacetoxylupa-12,18- | 1,3-dioxol- | Ac | 52.2 | |
| diene and 1,3-dioxol-5-ylurazole | 5-yl | | | |
| Positive control: Amphotericine B (1 μM) | | | 55.4 | |
| Negative control: Broth + DMSO | | | 0.0 | |

### Example 34

### Cytotoxicity Tests of the Betulin Derived Compounds

Caco-2 cells (cell line used as a model for human intestine) were introduced in a 96 well plate in an amount of 35 000 cells (for LDH method), 45 000 cells (for WST-1 method), or 25 000 cells (for ATP method) per well. After proliferation for 24 hours, the cells were exposed to the compounds being tested for 24 hours by adding said compounds to the cultivation medium to give a concentration of 500 mM (as stock solutions in DMSO).

The influence of the compounds on the viability of the cells, was measured by three different methods. Polymyxin B was used as the control. Lactate dehydrogenase (LDH) is an enzyme found in cells, and accordingly, increased amounts thereof outside cells result from cell membrane damage. The amount of LDH in the sample due to exposure was quantified by means of an enzymatic reaction using the INT (iodonitrotetrazolium) colour reagent wherein the coloured reaction product formed was determined photometrically at 490 nm. In the WST-1 method, the metabolic activity of the cells after exposure was measured using the WST-1 reagent. Metabolic activity of a cell results in the generation of a coloured product with the reagent, said product being then used to evaluate the viability of the cells by photometric measurements (absorbance at 440 nm). In the ATP method, the amount of ATP within cells decreasing rapidly due to cellular damage was measured. In the method, ATP was luminometrically quantified by means of the ATP dependent luciferase-luciferin reaction.

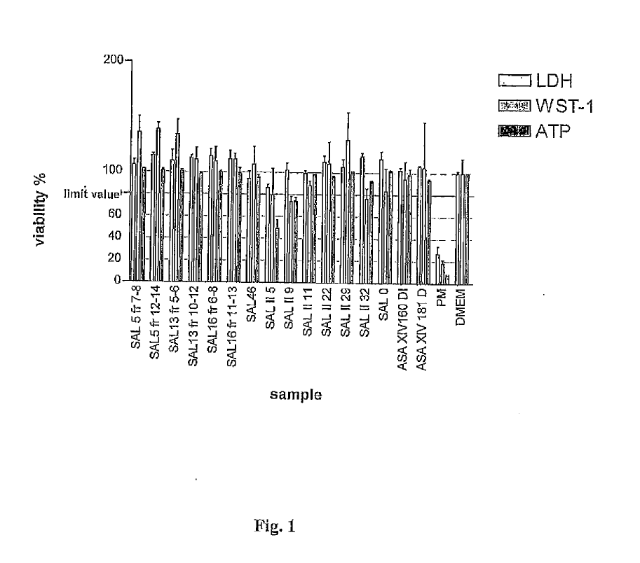

Appended FIG. 1 shows effects on the viability of Caco-2 cells (%) after exposure for 24 hour as measured by three assay methods of cellular viability (LDH, WSR-1 and ATP methods). Compounds exceeding the limit value, i.e. 80% are considered to have no significant negative effect on the viability of cells is vitro. The compounds of the Table 6 below were used for testing.

TABLE 6

| Code | Compound |
|---|---|
| PM | positive control (polymyxin B sulfate) |
| Sal-5 fr. 7-8 | 3,28-O-isostearylic acid diester of betulin |
| Sal-5 fr. 12-14 | 28-O-isostearylic acid ester of betulin |
| Sal-13 fr. 5-6 | 3,28-O-oleic acid diester of betulin |
| Sal-13 fr. 10-12 | 28-O-oleic acid ester of betulin |
| Sal-16 fr. 6-8 | 3,28-O-octanylic acid diester of betulin |
| Sal-16 fr. 11-13 | 28-O-octanylic acid ester of betulin |
| Sal-46 | betulin 3,28-diacetate |
| Sal-II-5 | betulin 28-acetate |
| Sal-II-9 | betulin 3-oxo-28-acetate |
| Sal-II-11 | betulinic acid |
| Sal-II-22 | betulin 3-deoxo-2,3-didehydro |
| Sal-II-29 | betulin 3-deoxo-2,3-didehydro-28-acetate |
| Bal-II-32 | betulonic acid |
| Sal-0 | betulin |
| Asa-XIV-160-DI | 28-N-acetylanthranilic acid ester of betulin |
| Asa-XIV-181-D | 28-nicotinic acid ester of betulin |

1-52. (canceled)

53. Use of betulin derivatives of the general formula I and pharmaceutically acceptable salts thereof for the production of a medicament against leishmaniasis and protozoa of the Leishmania genus, where in formula I 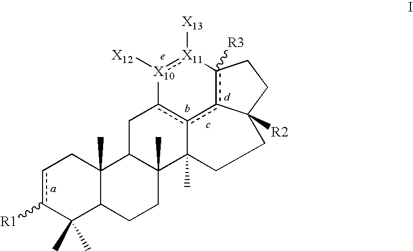
R1=OH;
R2=CH₂O(C═O)R_{f} where R_{f} C₁-C₂₂ linear or branched alkyl or alkenyl group;
R3=CH₂═CCH₃; and
X10=X11=H, X12=X13=absent, a, b, c, and d each represent a single bond and e is absent; or
R1═OH;
A. R2=CH₂O(C═O)CH₂(CHR_{g})COOY where R_{g}═H, C₁-C₂₂ linear or branched alkyl or alkenyl group, Y═H, Na, K, Ca, Mg, C₁-C₄-alkyl group or NRₕ where Rₕ═H or C₁-C₄-alkyl group;
B. R3=CH₂═CCH₃; and
C. X₁₀═X₁₁═H, X₁₂═X₁₃=absent, a, b, c, and d each represent a single bond and e is absent; or
R1=OH;
R2=CH₂ORᵢ where Rᵢ=2,5-diaminopentanoyl, 2-(acetylamino)benzoyl or N,N,N-trimethyl-2-oxoethanaminium group;
R3=CH₂═CCH₃; and
X₁₀═X₁₁═H, X₁₂═X₁₃=absent, a, b, c, and d each represent a single bond and e is absent; or
R1=OH;
R2=CH₂ORₙ or CH₂O(C═O)CH₂OR′ where R′=verbenyl, terpinyl, thymyl, carvacryl, menthyl, cinnamyl, curcuminyl, eugenyl, bornyl, isobornyl, longifolyl, isolongifolyl, globulyl, epiglobulyl, cedryl, or epicedryl group and Rₙ=chrysanthemoyl or retinoyl group; and
R3=CH₂═CCH₃;
X₁₀═X₁₁═H, X₁₂═X₁₃=absent, a, b, c, and d each represent a single bond and e is absent; or
R1=O(C═O)Rₘ where Rₘ═C₁-C₂₂ linear or branched alkyl or alkenyl group;
R2=CH₂O(C═O)Rₒ where Rₒ═C₁-C₂₂ linear or branched alkyl or alkenyl group;
R3=CH₂═CCH₃; and
X₁₀═X₁₁═H, X₁₂═X₁₃=absent, a, b, c, and d each represent a single bond and e is absent; or
R1=O(C═O)CH₂(CHR₂)COOY where R_{c}═C₁-C₂₂ linear or branched alkyl or alkenyl group, Y═H, Na, K, Ca, Mg, C₁-C₄ alkyl group or NRₕ where Rₕ═H or a C₁-C₄ alkyl group;
R2=CH₂O(C═O)CH₂(CHR_{d})COOY where R_{d}═H, C₁-C₂₂ linear or branched alkyl or alkenyl group, Y═H, Na, K, Ca, Mg, C₁-C₄ alkyl group or NRₖ where Rₖ═H or a C₁-C₄ alkyl group;
R3=CH₂═CCH₃; and
X₁₀═X₁₁═H, X₁₂═X₁₃=absent, a, b, c, and d each represent a single bond and e is absent; or
R1=ORᵣ where Rᵣ=2,5-diaminopentanoyl, 2-(acetylamino)benzoyl or N,N,N-trimethyl-2-oxoethanaminium group;
R2=CH₂ORₚ where Rₚ=2,5-diaminopentanoyl, 2-(acetylamino)benzoyl or N,N,N-trimethyl-2-oxoethanaminium group;
R3=CH₂═CCH₃; and
X₁₀═X₁₁═H, X₁₂═X₁₃=absent, a, b, c, and d each represent a single bond e is absent; or
R1=ORᵥ or O(C═O)CH₂OR′ where R′=verbenyl, terpinyl, thymyl, carvacryl, menthyl, cinnamyl, curcuminyl, eugenyl, bornyl, isobornyl, longifolyl, isolongifolyl, globulyl, epiglobulyl, cedryl or epicedryl group and Rᵥ=chrysantemoyl or retinoyl group
R2=CH₂ORᵤ or CH₂O(C═O)CH₂OR′ where R′=verbenyl, terpinyl, thymyl, carvacryl, menthyl, cinnamyl, curcuminyl, eugenyl, bornyl, isobornyl, longifolyl, isolongifolyl, globulyl epiglobulyl, cedryl or epicedryl group and Rᵤ=chrysantemoyl or retinoyl group;
R3=CH₂═CCH₃; and
X₁₀═X₁₁═H, X₁₂═X₁₃=absent, a, b, c, and d each represent a single bond and e is absent; or
R1=OH;
R2=(C═O)NHCHRₓCOOY where Y═H, Na, K, Ca, Mg, C₁-C₄ alkyl group or NR_{y} where R_{y}═H or a C₁-C₄ alkyl group and Rₓ═H, C₁-C₄ alkyl, benzyl, 4-hydroxybenzyl, 4-imidazolylmethyl or 3-indolylmethyl group or L-aspartate, L-histidine, L-glutamine or L-lysine residue;
R3=CH₂═CCH₃; and
X₁₀═X₁₁═H, X₁₂═X₁₃=absent, a, b, c, and d each represent a single bond and e is absent; or
R1=oxo group;
R2=(C═O)NHCHRₓCOOY where Y═H, Na, K, Ca, Mg, C₁-C₄ alkyl group or NR_{y} where R_{y}═H or a C₁-C₄ alkyl group and Rₓ═H, C₁-C₄ alkyl, benzyl, 4-hydroxybenzyl group, CH₂CH₂CH₂CH₂NH₂, 4-imidazolylmethyl, 3-indolylmethyl, CH₂COOZ or CH₂CH₂COOZ group and Z═R_{y};
R3=CH₂═CCH₃; and
X₁₀═X₁₁═H, X₁₂═X₁₃=absent, a, b, c, and d each represent a single bond and e is absent; or
R1=oxo group;
R2=(C═O)R_{w}, where R_{w}=verbenyl, terpinyl, thymyl, carvacryl, menthyl, cinnamyl, curcuminyl, eugenyl, bornyl, isobornyl, longifolyl, isolongifolyl, globulyl, epiglobulyl, cedryl or epicedryl group;
R3=CH₂═CCH₃; and
X₁₀═X₁₁═H, X₁₂═X₁₃=absent, a, b, c, and d each represent a single bond and e is absent; or
R1=OH or O—(C═O)R_{b} where R_{b}═C₃-C₈ cyclic or heterocyclic residue, substituted or unsubstituted phenyl or benzyl residue or C₁-C₂₂ alkyl or alkenyl group; R2=CH₂OH or CH₂O—(C═O)R_{f} where R_{f}═C₃-C₈ cyclic or heterocyclic residue, substituted or unsubstituted phenyl or benzyl residue or C₁-C₂₂ alkyl or alkenyl group;
R3=H₂C═CCH₂R_{q} or CH₃CCH₂R_{q} where R_{q}=3-dihydrofuran-2,5-dione or 3-pyrrolidine-2,5-dione or CH(COORₒ)CH₂COOR_{z} where Rₒ═H, Na, K, Ca, Mg or a C₁-C₂₂ linear or branched alkyl or alkenyl group and R_{z}═H, Na, K, Ca, Mg or a C₁-C₂₂ linear or branched alkyl or alkenyl group; and
X₁₀═X₁₁═H, X₁₂═X₁₃=absent, a, b, c, and d each represent a single bond and e is absent; or
R1=H, OR_{z}, O(C═O)R_{b}, NRₐR_{Z}, CN, ═NORₐ, CHO, (C═O)OR_{z}, SR_{z}, ═O or ═S where R_{z}═H, C₁-C₆ linear or branched alkyl or alkenyl group or an aromatic group ZZ shown below and Rₐ═H, C₁-C₆ linear or branched alkyl or alkenyl group or an aromatic group ZZ, and R_{b}═H, C₁-C₂₂ linear or branched alkyl or alkenyl group or an aromatic group ZZ or R1 corresponds to the partial structure XX shown below;
R2=CH₂OR_{z}, CH₂O(C═O)R_{b}, (C═O)OR_{b}, CH₂NRₐR_{z}, CH₂CN, CN, CH═NORa, CH₂CHO, CH₂(C═O)OR_{z}, CH₂SR_{Z}, CH═O or CH═S where R_{z}═H, C₁-C₆ linear or branched alkyl or alkenyl group or an aromatic group ZZ and Rₐ═H, C₁-C₆ linear or branched alkyl or alkenyl group or an aromatic group ZZ and R_{b}═H, C₁-C₂₂ linear or branched alkyl or alkenyl group or an aromatic group ZZ or R2 corresponds to the partial structure YY shown below;
R3=CH₂═C—CH₃ or CH₃—CH—CH₃; and
X₁₀═X₁₁═H, X₁₂═X₁₃=absent, a, b, c, and d independently represent a single or a double bond and e is absent;
said partial structures XX and YY where YY═CH₂XX being selected from the group consisting of: 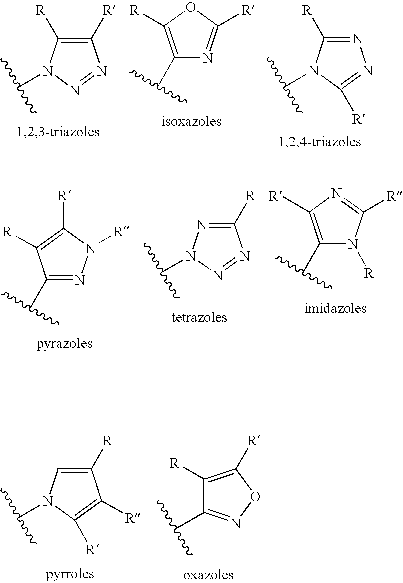
in which structures R, R′, and R″ independently represent H, an aromatic group ZZ, C₁-C₆ linear or branched alkyl or alkenyl group, the aromatic group ZZ being of the form: 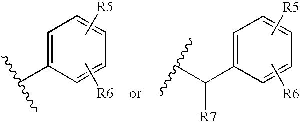
where R5, R6 and/or R7 is H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen, nitro, carboxy, carboxyl, acetyl, R5-R6 forms a cyclic methylenedioxy group, sulfate, cyano, hydroxy or trifluoromethyl group; or
R1=H, OR_{z}, NRₐR_{z}, CN, CHO, (C═O)OR_{z}, O(C═O)R_{b}, O(C═O)NHR_{f}, SR_{z}, ═O or ═S where R_{z}═H, C₁-C₆ linear or branched alkyl or alkenyl group or an aromatic group ZZ and Rₐ═H, C₁-C₆ linear or branched alkyl or alkenyl group or an aromatic group ZZ and R_{b}═H, C₁-C₂₂ linear or branched alkyl or alkenyl group or an aromatic group ZZ or R_{b} corresponds to the partial structure YX shown below and R_{f}═H, C₁-C₆ linear or branched alkyl or alkenyl group or an aromatic group ZZ or R_{f} corresponds to the partial structure YX shown below;
R2=CH₂OR_{z}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{f}, CH₂SR_{z}, CH═O or CH═S where R_{z}═H, C₁-C₆ linear or branched alkyl or alkenyl group or an aromatic group ZZ and Rₐ═H, C₁-C₆ linear or branched alkyl or alkenyl group, or an aromatic group ZZ and R_{b}═H, C₁-C₂₂ linear or branched alkyl or alkenyl group or an aromatic group ZZ or R_{b} corresponds to the partial structure YX shown below and R_{f}═H, C₁-C₆ linear or branched alkyl or alkenyl group or an aromatic group ZZ or R_{f} corresponds to the partial structure YX shown below;
R3=CH₂═C—CH₃ or CH₃—CH—CH₃; and
X₁₀═X₁₁═H, X₁₂═X₁₃=absent, a, b, c, and d independently represent a single or a double bond and e is absent;
said aromatic group ZZ being of the form: 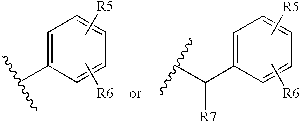
where R5, R6 and/or R7 is H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen, nitro, carboxy, carboxyl, acetyl, R5-R6 forms a cyclic methylenedioxy group, sulfate, cyano, hydroxy or trifluoromethyl; and
the partial structure R_{f} or R_{b} is of the form YX: 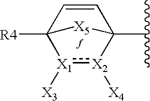
where R4=H or a C₁-C₂₀ linear or branched alkyl or alkenyl group or an aromatic group ZZ, X₅=absent, C, O, N or S, X₁-X₂ forms a cyclic partial structure of the form: X₁—(X₃═X₆)—X₇—(X₄═X₈)—X₂ where X₁═X₂═C or N; X₃═X₄═C; X₆═X₈═O, S or absent, X₇═C, O, S, or N—X₉ where X₉═H, C₁-C₆ linear or branched alkyl or alkenyl group or an aromatic group ZZ and f is a single or a double bond; or
R1=H, OR, NRₐR_{z}, CN, CHO, (C═O)OR_{z}, O(C═O)R_{b}, O(C═O)NHR_{z}, SR_{z}, ═O or ═S where R_{z}═H, C₁-C₆ linear or branched alkyl or alkenyl group or an aromatic group ZZ and Rₐ═H, C₁-C₆ linear or branched alkyl or alkenyl group or an aromatic group ZZ and R_{b}═H, C₁-C₂₂ linear or branched alkyl or alkenyl group or an aromatic group ZZ;
R2=CH₂OR_{z}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{z}, CH₂SR_{z}, CH═O or CH═S where R_{z}═H, C₁-C₆ linear or branched alkyl or alkenyl group or an aromatic group ZZ and Rₐ═H, C₁-C₆ linear or branched alkyl or alkenyl group or an aromatic group ZZ and R_{b}═H, C₁-C₂₂ linear or branched alkyl or alkenyl group or an aromatic group ZZ;
R3=CH₂═C—CH₃ or CH₃—CH—CH₃; and
ZZ being of the form: 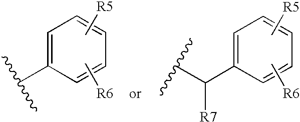
where R5, R6 and/or R7 is H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen, nitro, carboxy, acetyl, R5-R6 forms a cyclic methylenedioxy group, sulfate, cyano, hydroxy or trifluoromethyl, at X₁₀-X₁₁ a cyclic or heterocyclic partial structure having the form X₁₀—(X₁₂═X₁₄)—X₁₅—(X₁₃═X₁₆)—X₁₁ is present where X₁₀═X₁₁═C or N, X₁₂═X₁₃═C, X₁₄═X₁₆═O, S or absent, X₁₅═C, O, S or N—X₁₇ where X₁₇═H, a C₁-C₆ linear or branched alkyl or alkenyl group or an aromatic group ZZ, and a, b, c, d and e independently represent double or single bonds; or
betulin 28-acetic acid methyl ester, 20,29-dihydrobetulonic acid, betulonic acid, betulonic alcohol, 3-deoxy-2,3-dihydrobetulin, betulin 28-oxime, betulin 3,28-dioxime, betulin 3-acetoxyoxime-28-nitrile;
with the provision that the compound is not betulin, betulinic acid, betulinic aldehyde or dihydrobetulinic acid.

54. Use according to claim 53, characterized in that the betulin derivative is selected from the group consisting of betulonic alcohol 28-acetate, betulonic acid 28-methylester, betulin 3,28-dioxime, betulin 28-oxime, betulonic alcohol, betulin 3-acetoxime-28-nitrile, betulin 28-acetic acid methylester, 20,29-dihydrobetulonic acid, betulonic acid, 28-aspartateamide dimethylester of betulonic acid, betulin 28-N-acetylanthranilic acid ester, Diels-Alder adduct of 3β,28-diacetoxylupa-12,18-diene and urazole, Diels-Alder adduct of 3β,28-diacetoxylupa-12,18-diene and 4-methylurazole, Diels-Alder adduct of 3β,28-diacetoxylupa-12,18-diene and 4-phenylurazole, Diels-Alder adduct of 3β,28-diacetoxylupa-12,18-diene and p-fluoro-4-phenylurazole, Diels-Alder adduct of 3β,28-diacetoxylupa-12,18-diene and m-methoxy-4-phenylurazole, Diels-Alder adduct of 3β,28-diacetoxylupa-12,18-diene and m-acetoxy-4-phenylurazole, Diels-Alder adduct of 3β,28-diacetoxylupa-12,18-diene and 1-naphthylurazole, and Diels-Alder adduct of 3β,28-diacetoxylupa-12,18-diene and 1,3-dioxol-5-ylurazole.

55. A betulin derivative of the general formula I′, or a pharmaceutically acceptable salt thereof, where in formula I′ 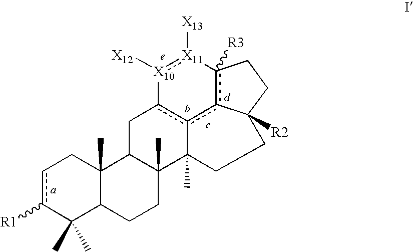
D. R1=OH;
E. R2=CH₂O(C═O)CH₂(CHR_{g})COOY where R_{g}═C₄-C₂₂ linear or branched alkyl or alkenyl group, Y═H, Na, K, Ca, Mg, C₁-C₄-alkyl group or NRₕ where Rₕ═H or C₁-C₄-alkyl group;
F. R3=CH₂═CCH₃; and
G. X₁₀═X₁₁═H, X₁₂═X₁₃=absent, a, b, c, and d each represent a single bond and e is absent; or
R1=OH;
R2=CH₂ORᵢ where Rᵢ=2,5-diaminopentanoyl, 2-(acetylamino)benzoyl, N,N,N-trimethyl-2-oxoethanaminium or isostearyl group;
R3=CH₂═CCH₃; and
X₁₀═X₁₁═H, X₁₂═X₁₃=absent, a, b, c, and d each represent a single bond and e is absent; or
R1=OH;
R2=CH₂ORₙ or CH₂O(C═O)CH₂OR′ where R′=verbenyl, terpinyl, thymyl, carvacryl, menthyl, cinnamyl, curcuminyl, eugenyl, bornyl, isobornyl, longifolyl, isolongifolyl, globulyl, epiglobulyl, cedryl or epicedryl group and Rᵤ=retinoyl group; and;
R3=CH₂═CCH₃; and
X₁₀═X₁₁═H, X₁₂═X₁₃=absent, a, b, c, and d each represent a single bond and e is absent; or
R1=O(C═O)CH₂(CHR_{c})COOY where R_{c}═C₄-C₂₂ linear or branched alkyl or alkenyl group, Y═H, Na, K, Ca, Mg, C₁-C₄ alkyl group or NRₕ where Rₕ═H or a C₁-C₄ alkyl group;
R2=CH₂O(C═O)CH₂(CHR_{d})COOY where R_{d}═C₄-C₂₂ linear or branched alkyl or alkenyl group, Y═H, Na, K, Ca, Mg, C₁-C₄ alkyl group or NRₖ where Rₖ═H or a C₁-C₄ alkyl group;
R3=CH₂═CCH₃; and
X₁₀═X₁₁═H, X₁₂═X₁₃=absent, a, b, c, and d each represent a single bond and e is absent; or
R1=ORᵣ where Rr=2,5-diaminopentanoyl, 2-(acetylamino)benzoyl, N,N,N-trimethyl-2-oxoethanaminium or isostearoyl group;
R2=CH₂ORₚ where Rₚ=2,5-diaminopentanoyl, 2-(acetylamino)benzoyl, N,N,N-trimethyl-2-oxoethanaminium or isostearoyl group
R3=CH₂═CCH₃; and
X₁₀═X₁₁═H, X₁₂═X₁₃=absent, a, b, c, and d each represent a single bond and e is absent; or
R1=ORᵥ or O(C═O)CH₂OR′ where R′=verbenyl, terpinyl, thymyl, carvacryl, menthyl, cinnamyl, curcuminyl, eugenyl, bornyl, isobornyl, longifolyl, isolongifolyl, globulyl, epiglobulyl, cedryl or epicedryl group and retinoyl group
R2=CH₂ORᵤ or CH₂O(C═O)CH₂OR′ where R′=verbenyl, terpinyl, thymyl, carvacryl, menthyl, cinnamyl, curcuminyl, eugenyl, bornyl, isobornyl, longifolyl, isolongifolyl, globulyl epiglobulyl, cedryl or epicedryl group and Rᵤ=retinoyl group;
R3=CH₂═CCH₃; and
X₁₀═X₁₁═H, X₁₂═X₁₃=absent, a, b, c, and d each represent a single bond and e is absent; or
R1=OH;
R2=(C═O)NHCHRₓCOOY where Y═H, Na, K, Ca, Mg, C₁-C₄ alkyl group or NR_{y} where R_{y}═H or a C₁-C₄ alkyl group and Rₓ═CH₂CH₂CH₂CH₂NH₂ or 4-imidazolylmethyl group;
R3=CH₂═CCH₃; and
X₁₀═X₁₁═H, X₁₂═X₁₃=absent, a, b, c, and d each represent a single bond and e is absent; or
R1=oxo group;
R2=(C═O)NHCHRₓCOOY where Y═H, Na, K, Ca, Mg, C₁-C₄ alkyl group or NR_{y} where R_{y}═H or a C₁-C₄ alkyl group and Rₓ═CH₂CH₂CH₂CH₂NH₂, 4-imidazolylmethyl, 3-indolylmethyl CH₂COOZ or CH₂CH₂COOZ group and Z═R_{y};
R3=CH₂═CCH₃; and
X₁₀═X₁₁═H, X₁₂═X₁₃=absent, a, b, c, and d each represent a single bond and e is absent; or
R1=oxo group;
R2=(C═O)OR_{w} where R_{w}=verbenyl, terpinyl, thymyl, carvacryl, menthyl, cinnamyl, curcuminyl, eugenyl, bornyl, isobornyl group, longifolyl, isolongifolyl, globulyl, epiglobulyl, cedryl or epicedryl group;
R3=CH₂═CCH₃; and
X₁₀═X₁₁═H, X₁₂═X₁₃=absent, a, b, c, and d each represent a single bond and e is absent; or
R1=OH or O—(C═O)R_{b} where R_{b}═C₃-C₈ cyclic or heterocyclic residue, substituted or unsubstituted phenyl or benzyl residue, C₁-C₂₂ alkyl or alkenyl group;
R2=CH₂OH or CH₂O—(C═O)R_{f} where R_{f}═C₃-C₈ cyclic or heterocyclic residue, substituted or un substituted phenyl or benzyl residue, C₁-C₂₂ alkyl or alkenyl group;
R3=H₂C═CCH₂R_{q} or CH₃CCH₂R_{q} where R_{q}=3-dihydrofuran-2,5-dione, 3-pyrrolidine-2,5-dione or CH(COORₒ)CH₂COOR_{z} where Rₒ═H, Na, K, Ca, Mg or a C₁-C₂₂ linear or branched alkyl or alkenyl group and R_{z}═H, Na, K, Ca, Mg or a C₁-C₂₂ linear or branched alkyl or alkenyl group; and X₁₀═X₁₁═H, X₁₂═X₁₃=absent, a, b, c, and d each represent a single bond and e is absent; or
R1=H, OR_{z}, O(C═O)R_{b}, NRₐR_{z}, CN, ═NORₐ, CHO, (C═O)OR_{z}, SR_{z}, ═O or ═S where R_{z}═H, C₁-C₆ linear or branched alkyl or alkenyl group or an aromatic group ZZ shown below and Rₐ═H, C₁-C₆ linear or branched alkyl or alkenyl group or an aromatic group ZZ and R_{b}═H, C₁-C₂₂ linear or branched alkyl or alkenyl group or an aromatic group ZZ or R1 corresponds to the partial structure XX shown below;
R2=CH₂OR_{z}, CH₂O(C═O)R_{b}, (C═O)OR_{b}, CH₂NRₐR_{z}, CH₂CN, CN, CH═NORₐ, CH₂CHO, CH₂(C═O)OR_{z}, CH₂SR_{z}, CH═O or CH═S where R_{z}═H, C₁-C₆ linear or branched alkyl or alkenyl group or an aromatic group ZZ and Rₐ═H, C₁-C₆ linear or branched alkyl or alkenyl group or an aromatic group ZZ and R_{b}═H, C₁-C₂₂ linear or branched alkyl or alkenyl group or an aromatic group ZZ or R2 corresponds to the partial structure YY shown below, with the proviso that R1 or R2 comprises the group XX;
R3=CH₂═C—CH₃ or CH₃—CH—CH₃;
X₁₀═X₁₁═H, X₁₂═X₁₃=absent, a, b, c, and d independently represent a single or a double bond and e is absent;
the partial structures XX and YY where YY═CH₂XX being selected from the group consisting of: 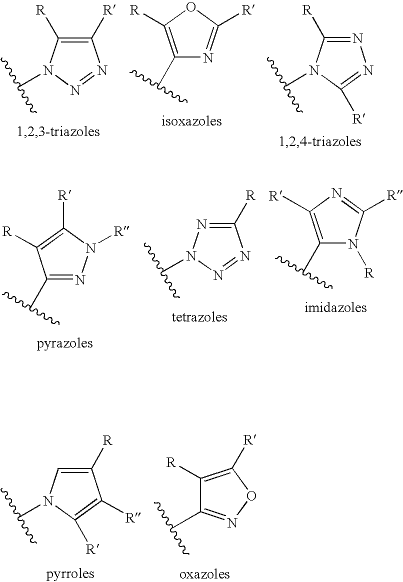
in which structures R, R′, and R″ independently represent H, an aromatic group ZZ, C₁-C₆ linear or branched alkyl or alkenyl group, the aromatic group ZZ being of the form: 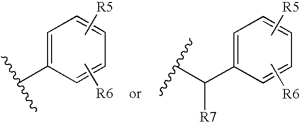
where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen, nitro, carboxy, carboxyl, acetyl, R5-R6 forms a cyclic methylenedioxy group, sulfate, cyano, hydroxy or trifluoromethyl group; or
R1=H, OR_{z}, NRₐR_{z}, CN, CHO, (C═O)OR_{z}, O(C═O)R_{b}, O(C═O)NHR_{f}, SR_{z}, ═O or ═S where R_{z}═H, C₁-C₆ linear or branched alkyl or alkenyl group or an aromatic group ZZ and Rₐ═H, C₁-C₆ linear or branched alkyl or alkenyl group or an aromatic group ZZ and R_{b}═H, C₁-C₂₂ linear or branched alkyl or alkenyl group or an aromatic group ZZ or R_{b} corresponds to the partial structure YX shown below and R_{f}═H, C₁-C₆ linear or branched alkyl or alkenyl group or an aromatic group ZZ or R_{f} corresponds to the partial structure YX shown below;
R2=CH₂OR_{z}, (C═O)OR_{b}, CH₂NRₐR_{z}, CH₂CN, CH₂CHO, C₁₋₁₂(C═O)OR_{z}, CH₂O(C═O)R_{b}, CH₂O(C═O)NHR_{f}, CH₂SR_{z}, CH═O or CH═S where R_{z}═H, C₁-C₆ linear or branched alkyl or alkenyl group or an aromatic group ZZ and Rₐ═H, C₁-C₆ linear or branched alkyl or alkenyl group, or an aromatic group ZZ and R_{b}═H, C₁-C₂₂ linear or branched alkyl or alkenyl group or an aromatic group ZZ or R_{b} corresponds to the partial structure YX shown below and R_{f}═H, C₁-C₆ linear or branched alkyl or alkenyl group or an aromatic group ZZ or R_{f} corresponds to the partial structure YX shown below, with the proviso that R1 or R2 comprises the group YX;
R3=CH₂═C—CH₃ or CH₃—CH—CH₃;
X₁₀═X₁₁═H, X₁₂═X₁₃=absent, a, b, c, and d independently represent a single or a double bond; and e is absent, said aromatic group ZZ being of the form: 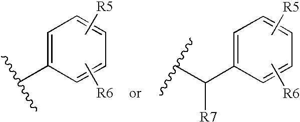
where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen (fluoro, chloro, bromo, iodo), nitro, carboxy, carboxyl, acetyl, R5-R6 forms a cyclic methylenedioxy group, sulfate, cyano, hydroxy or trifluoromethyl group; and
the partial structure R_{f} or R_{b} is of the form YX: 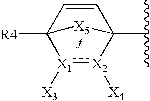
where R4=H or a C₁-C₂₀ linear or branched alkyl or alkenyl group or an aromatic group ZZ, X₅=absent, C, O, N or S, X₁-X₂ forms a cyclic partial structure of the form: X₁—(X₃═X₆)—X₇—(X₄═X₈)—X₂ where X₁═X₂═C or N, X₃═X₄═C, X₆═X₈═O, S or absent, X₇═C, O, S or N—X₉ where X₉=H, C₁-C₆ linear or branched alkyl or alkenyl group or an aromatic group ZZ, and f is a single or a double bond; or
R1=H, OR, NRₐR_{z}, CN, CHO, (C═O)OR_{z}, O(C═O)R_{b}, O(C═O)NHR_{z}, SR_{z}, ═O or ═S where R_{z}═H, C₁-C₆ linear or branched alkyl or alkenyl group or an aromatic group ZZ and Rₐ═H, C₁-C₆ linear or branched alkyl or alkenyl group or an aromatic group ZZ and R_{b}═H, C₁-C₂₂ linear or branched alkyl or alkenyl group or an aromatic group ZZ;
R2=CH₂OR_{z}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{z}, CH₂SR_{Z}, CH═O or CH═S where Rₐ═H, C₁-C₆ linear or branched alkyl or alkenyl group or an aromatic group ZZ and Rₐ═H, C₁-C₆ linear or branched alkyl or alkenyl group or an aromatic group ZZ and R_{b}═H, C₁-C₂₂ linear or branched alkyl or alkenyl group or an aromatic group ZZ;
R3=CH₂═C—CH₃ or CH₃—CH—CH₃;
ZZ being of the form: 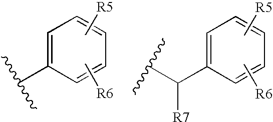
where R5, R6 and/or R7 is H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen, nitro, carboxy, carboxyl, acetyl, R5-R6 forms a cyclic methylenedioxy group, sulfate, cyano, hydroxy or trifluoromethyl, at X₁₀-X₁₁, a cyclic or heterocyclic partial structure having the form X₁₀—(X₁₂═X₁₄)—X₁₅—(X₁₃═X₁₆)—X₁₁ is present where X₁₀═X₁₁═C or N, X₁₂═X₁₃═C, X₁₄═X₁₆═O, S or absent, X₁₅═C, O, S or N—X₁₇ where X₁₇═H, a C₁-C₆ linear or branched alkyl or alkenyl group or an aromatic group ZZ, with the proviso that X₁₇ is not phenyl; and a, b, c, d and e independently represent double or single bonds; or
betulin 28-acetic acid methyl ester.

56. Betulin derivative according to claim 55, characterized in that the betulin derivative is selected from the group consisting, betulin 28-acetic acid methyl ester, 28-aspartateamide dimethyl ester of betulonic acid, Diels-Alder adduct of 3β,28-diacetoxylupa-12,18-diene and urazole, Diels-Alder adduct of 3β,28-diacetoxylupa-12,18-diene and 4-methylurazole, Diels-Alder adduct of 3β,28-diacetoxylupa-12,18-diene and p-fluoro-4-phenylurazole, Diels-Alder adduct of 3β,28-diacetoxylupa-12,18-diene and m-methoxy-4-phenylurazole, Diels-Alder adduct of 3β,28-diacetoxylupa-12,18-diene and 1-naphthylurazole, and Diels-Alder adduct of 3β,28-diacetoxylupa-12,18-diene and 1,3-dioxol-5-ylurazole.

57. Composition against protozoa of the genus Leishmania and against leishmaniasis, characterized in that said composition comprises 0.01 to 80% by weight of a betulin derivative according to claim 55 or 56, and optionally one or more agents selected from the group of adjuvants and excipients.
